(19) United States
(12) Patent Application Publication
(10) Pub. No.: US 2025/0135287 A1
(43) Pub. Date: May 1, 2025

(54) METHOD AND SYSTEM TO PROVIDE INDIVIDUALIZED INTERVENTIONS BASED ON A WELLNESS MODEL

(71) Applicant: LULULEMON ATHLETICA CANADA INC., Vancouver (CA)

(72) Inventors: Samuel Haskel BERGMANN-GOOD, Vancouver (CA); Joseph John SANTRY, Vancouver (CA); Peder Richard Douglas SANDE, Vancouver (CA); Todd James SMITH, Vancouver (CA); Gillian LAI, Vancouver (CA); Robert John GATHERCOLE, Vancouver (CA); Jordan Stuart GOLDFARB, Vancouver (CA); Daljinder Singh SANGHERA, Vancouver (CA)

(21) Appl. No.: 18/836,587

(22) PCT Filed: Feb. 22, 2023
(86) PCT No.: PCT/CA2023/050221

§ 371 Date: Aug. 7, 2024

Related U.S. Application Data
(60) Provisional application No. 63/314,200, filed on Feb. 25, 2022.

(51) Int. Cl. A63B 24/00; A61B 5/00; A61B 5/16; A63B 71/06; G16H 10/60; G16H 20/30; G16H 20/70; G16H 80/00

(52) U.S. Cl. CPC A63B 24/0075; A61B 5/165; A61B 5/7267; A63B 71/0622; G16H 10/60; G16H 20/30; G16H 20/70; G16H 80/00; A63B 2024/0065

(57) ABSTRACT

Embodiments described herein relate to systems and methods for an individualized intervention to generate and provide wellness pattern aware interventions. The system generates digital instructions and output for an online web application, device hosted application, smart device, or similar system. In one embodiment, the system provides the individualized intervention that matches device capacity, user context, and the best intervention type for a multi feel state longitudinal journey over a multi-day duration.

### CROSS-REFERENCE TO RELATED APPLICATION

The present application claims the benefit of and priority to U.S. Provisional Application No. 63/314,200 entitled METHOD AND SYSTEM TO PROVIDE INDIVIDUALIZED INTERVENTIONS BASED ON A WELLNESS MODEL filed Feb. 25, 2022, the entire contents of which are hereby incorporated by reference.

### FIELD

The present disclosure relates to methods and systems for electrical computers, digital processing, computer interfaces, smart mirrors, monitoring, physiological states, psychological and emotional wellness, machine learning, digital simulation, and wellness interventions.

### INTRODUCTION

Humans experience different feel-states which can be classified into various categories. Feel-states shift, sometimes rapidly, over the course of a minute, hour, day, week, month, year and lifetime. Factors such as specific activities, and a lack of activity, environmental factors such as lighting, temperature, texture of surfaces, health factors, sleep patterns, nutritional patterns, relationships, social, intellectual, emotional, interpersonal, and physical engagement, context to perceive experiences as meaningful, sense of accomplishment and achievement, the use of caffeine, alcohol, nootropics, performance enhancers, microdoses, meditation practices, gratitude practices, and the like are known to influence an individual's feel-state.

Wellness is understood as representing both prevalence of positively perceived feel-states and a perceived positive trajectory within feel-states over a duration. There are different wellbeing theories, as well as models, which suggest example underlying factors in what influences the generation of a feel-state and how an individual perceives an experienced feel-state in the context of a series of feel-states. Some of these theories are based on specific psychological attributes of the individual that affect how an experience is perceived. For example, the same social experience may be presented to an introverted individual and an extroverted individual, and may result in significantly different feel-states. Example theories of wellness include the importance of positioning peak, or positive, feel-states at certain relative time locations within an overall experience.

Habit is understood as a repeated behavioural pattern over time. A habit may be intentional, unintentional, and may contain a “good” or “bad” behaviour that the user wants to continue and develop, or disrupt and end. Smaller micro-habits may be combined into a larger habit, and a combination of habits can form highly functional wellness structures and influence the probability of experiencing various feel-states. A number of theories of habit formation and disruption have also indicated the importance of context and building in a sense of accomplishment. When repetitive habits are dominant in an individual's behavioural patterns, an intervention introducing variety, or an element of surprise, can also positively affect the individual's feel-state and overall willingness to maintain a habit on the long term.

Embodiments described herein involve automated computer systems for receiving inputs, constructing data objects, applying data models, calculating wellness values based on data related to an individual, similar individuals, communities, cohorts, and digital representations of groups of individuals and specific individuals or twins, and providing interventions to the user.

Embodiments described herein involve automated computer systems and machine learning systems for providing individualized interventions based on factors such as wellness models, the user's profile, activity, responses, history, intentions, context, habits, and preferences.

### SUMMARY

In an aspect, embodiments described herein provide systems, computer products, and methods for generating a wellness pattern aware intervention with non-transitory memory storing data related to wellness, users, individualization, and instructions executed by a hardware processor to generate one or more sets of intervention instructions. These instructions are then interpreted by an output device to activate or trigger one or more interventions, or provide the user with one or more interventions. In an aspect, embodiments described herein provide, to the user, an individualized wellness pattern aware intervention, also referred to as an intervention.

Embodiments described herein may provide a non-transitory computer readable medium with instructions stored thereon, that when executed by a hardware processor cause the processor to generate output data indicating an individualized wellness pattern aware intervention and provide a user device with the individualized wellness pattern aware intervention to activate, trigger or present the intervention. The processor generates the intervention by applying a plurality of computer models to an individualization data object generated by processing input data identifying qualities associated with the intervention. The instructions control the hardware processor to trigger measurements by one or more input devices to receive at least part of the input data.

In some embodiments, the processor generates the individualization data object by processing additional input data identifying qualities associated with the intervention from a simulation environment with one or more digital twins corresponding to one or more physical objects relating to the intervention.

In some embodiments, the additional input data comprises data relating to at least one of mood and emotion within the simulation environment, and wherein the measurements comprise data relating to physical physiological modeling.

In some embodiments, the plurality of computer models comprise a wellness pattern model, an individualization model, and an intervention model.

In some embodiments, the individualized wellness pattern aware intervention comprises a multi-feel state longitudinal journey over a multi-day time duration to create a specific type of user experience over the multi-day time duration.

In some embodiments, the individualized wellness pattern aware intervention comprises an intervention output that is associated with an intervention type.

In some embodiments, the intervention type comprises one or more of suggesting an activity, starting an activity, suggesting a change in a current activity, changing a current activity, suggesting a change in the intensity of an activity, changing the intensity of an activity, canceling a scheduled activity, suggesting canceling a scheduled activity, suggesting the end of a current activity, ending a current activity, providing a reward, increasing a challenge, suggesting a product, providing a product, providing points, suggesting a future activity, starting a future activity, displaying a tool for user reflection, suggesting user feedback, displaying a tool for user feedback, suggesting a user chat or conversational activity, opening a user chat, suggesting an accountability partner, providing an accountability partner, suggesting a change in a current accountability partner, changing a current accountability partner, suggesting a team, providing a team, suggesting a change in a current team, changing a current team, suggesting a community, providing a community, suggesting a change in a current community, changing a current community, providing a level, removing a level, customizing a level, upgrading a level, downgrading a level, providing access to a product, providing access to an event, providing access to an experience, providing a badge, changing a badge, changing a social ranking, changing an indication of social belonging, changing an indication of contribution to a group, team or community, changing a social network connection, changing a group membership, providing a social connection, providing a means of communicating with another user, providing a means of sharing an emoticon with another user, providing feedback or an indication related to at least one of an activity being started, an activity in progress, an activity successfully completed, an activity duration, a user activity measure, a user activity success measure, a user activity partial success measure, a user activity in the context of a user's previous activity, a user activity in the context of a community activity, a user activity in the context of a digital twin data model, a user activity in the context of an ideal self data model, a user activity in the context of a cohort activity, a user activity in the context of a hero activity, a user activity in the context of a group of fitness class participants, a user activity in the context of an accountability partner, a user activity in the context of team, a user activity in the context of a community, a user's readiness to advance in an activity, a user's readiness to add a new activity, a user's readiness to stop an activity, a user's readiness to disrupt an activity, providing feedback or an indication related to at least one of a user habit being started, a user habit in progress, a user habit successfully completed, a user habit duration, a user habit activity measure, a user habit success measure, a user habit partial success measure, a user habit streak, a suggestion for resetting a habit, a suggestion for rescheduling a habit, rescheduling an activity, scheduling an activity, canceling a scheduled activity, a user's readiness to expand a habit, a user's readiness to add a new habit, a user's need to disrupt a habit, a user habit in the context of a community habit, a user habit in the context of a digital twin data model, a user habit in the context of an ideal self data model, a user habit in the context of a cohort habit, a user habit in the context of a hero habit, a user habit in the context of a group of fitness class participants, providing a virtual assistant, customizing a virtual assistant, providing information about a product, providing personalized information about a product, providing customized information about a product, providing a navigational path, providing a customized navigational path, providing a category of products, providing a customized category of products, and filtering a set of products, providing a special offer.

In some embodiments, the processor provides one or more media of the type video, interactive presentation, game, image, hologram image projection, autostereoscopic image projection, audio, text, spoken word, guided conversation, music, interactive simulation wherein the media contains content with a greater than average statistical probability to result in one or more of a technique correction, an emotional shift, an intellectual reframing of an experience, an emotional reframing of an experience, a distraction from a current or past experience, changing the user's anxiety level, changing the user's fear level, changing the user's hopefulness level, changing the user's sense of competence, changing the user's curiosity level, changing the user's feeling of agency, offering a relatable motivational experience, changing the user's competitiveness level, changing the user's cooperation level, changing the user's perception of social connection, changing the user's perception of personal advancement, changing the user's perception of social status, changing the user's perception of social belonging, changing the user's gratitude level, changing the user's calmness level, changing the user's focus level, changing the user's equanimity level, changing the user's sense of inner peace level.

In some embodiments, the multi-feel state longitudinal journey designed to create a specific type of user experience over a multi-day time duration is associated with one or more of the archetypal pattern wherein the archetypal pattern is one of, a quest for identity, a quest to find a ideal location, emotional state, or sense of spiritual realization, a quest for justice, a quest to help a community member, a quest for social connection, a quest for social acceptance within a group, a quest for a role or status designation, a quest in search of knowledge, competence or skill, a quest for acceptance and personal affirmation, a quest for transformation, a quest for self-actualization, a quest for pleasure, fun, surprise and adventure, a quest to remove a danger, a quest for a symbolic or metaphoric goal.

In some embodiments, the multi-feel state longitudinal journey designed to create a specific type of user experience over a multi-day time duration is associated with a pattern for adherence to a wellness, fitness lifestyle, basic health criteria, training plan, nutritional plan, and interventions are provided that are consistent with generating and improving the individual's adherence to the overall plan.

In some embodiments, the individualized wellness pattern aware intervention contains an interactive element or selectable indicia that enable a user to engage with and/or select from more than one individualized wellness pattern aware intervention.

In some embodiments, the individualized wellness pattern aware intervention is generated as a module with executable instructions to receive inputs, provide outputs and display the habit aware intervention.

In some embodiments, there is provided a computer system for providing a user device with an individualized wellness pattern aware intervention for a specific type of multi-feel state longitudinal journey over a time duration. The system a communication interface to transmit the individualized wellness pattern aware intervention; one or more non-transitory memory; a hardware processor programmed with executable instructions for generating the individualized wellness pattern aware intervention. The hardware processor: receives individualization input data associated with a user; generates an individualization data object using the input data; determines one or more intervention types to generate based on the data object by applying a plurality of computer models to the data object; generates one or more individualized wellness pattern aware intervention using the intervention types; one or more input devices to perform measurements for at least part of the input data; a user device comprising a hardware processor, and an interface to receive the intervention, and activate, trigger or present the intervention.

In some embodiments, the processor generates the individualization data object by processing additional input data identifying qualities associated with the intervention from a simulation environment with one or more digital twins corresponding to one or more physical objects relating to the intervention.

In some embodiments, the plurality of computer models comprise a wellness pattern model, an individualization model, and an intervention model.

In some embodiments, the user device provides a selectable indicia to accept the individualized wellness pattern aware intervention.

In some embodiments, the individualized wellness pattern aware intervention is activated, triggered or presented automatically at the user device.

In some embodiments, the individualization input data comprises data pertaining to one or more of user specified preference, user specified goal, user specific wellness objective, user specified training plan, user skill baseline, user wellness baseline, user personality baseline, user activity baseline, identifying a potential user cohort, identifying a potential user accountability partner, user habit, user activities, contextual data about user habit, contextual data about user activity, user fitness class history, user purchase history, user community participation, user exercise logs, user biometric records, user social media activity, user online activity.

In some embodiments, the one or more input devices comprise hardware components for receiving one or more of text entry, physiological metrics, biometric identifiers, video, audio, conversation, image, Global Positioning System (GPS) location, camera input, body mapping with sensors, mapping user position in a physical space, eye tracking, IoT (Internet of Things) input, web browsing history, social media history, purchase history, chat history, EEG signals, Heart Rate HR, Heart Rate Variability HRV, respiratory rate, blood glucose, oximetry rates, electrodermal activity, weight, body mass index BMI values, body temperature, PH levels.

In some embodiments, the one or more input devices further comprises one or more of a biometric sensor, physiological sensor, a room sensor, microphone, still camera, video camera, body-based sensor, smart mat based sensor, smart weight based, smart bike based sensor, smart glove based sensor, garment based sensor, smart footwear based sensor, augmented reality headset, virtual reality headset, metaverse headset, mixed reality device, virtual reality device, an augmented reality device, photodetector sensors, sweat sensors, and dehydrations sensors.

In some embodiments, the communication interface transmits to one or more output devices comprising one or more of a web application, an application installed on a user device, a smart mirror device, a connected music system, a connected lighting system, a connected exercise mat, a connected heating device, a connected cooling device, a connected smell diffuser device, a connected electrical stimulation device, a connected implanted medical device, a virtual reality headset, an augmented reality headset, a metaverse headset, a mixed reality device, a virtual reality devices, an augmented reality device, a haptic glove, a game controller, a haptic garment, a retail application, a coaching application, a fitness class or studio application, a meditation application, a retail application, a meal or nutritional supplement delivery service application, an email system application, a text message system application, notification system application, augmented reality environment, simulated reality environment, virtual reality environment, mixed reality environment, a game environment, a metaverse environment.

In some embodiments, the executable instructions configure the hardware processor to present, remove, or customize one or more of a retail offer, a retail experience, a user profile, user Wishlist, a workshop, coaching session, lecture, performance event, community event, an exercise class, an avatar, an avatar's apparel, a conversational interaction, a notification, a pop-up suggestion, an alarm, a badge, a reward, a number of points, a group membership.

In some embodiments, a data storage device with a repository of previously generated individualized wellness pattern aware interventions.

In some embodiments, the executable instructions evaluate a previously generated individualized wellness pattern aware intervention against one or more of context data, user data, community data, device capacity data, user preferences, connected device capacity data, availability of a community member data, availability of a cohort member data, availability of an accountability partner data, availability of team data and determine whether to regenerate, personalize, or augment the habit-aware intervention.

In some embodiments, the system provides an intervention within one or more of a fitness class environment, a studio environment, a class environment, a group activity environment, a performance environment, game environment, virtual reality system, simulated reality system, augmented reality system, mixed reality system, or metaverse system.

In some embodiments, the interface presents the intervention using one or more of a smart phone application, a web based application, computer based application, a smart mirror device, a smart watch, a item of smart jewelry, a item of smart apparel, a smart exercise bike, a smart gym, a smart weight, a smart lighting system, a smart audio system, a tablet, a computer, a device notification, a connected device such as a yoga mat, watch, heart rate monitor, breathing monitor, a blood glucose monitor, a galvanic skin response (GSR) monitor, an electronic implant, an EEG, a brain-computer interface. a hologram projection system, an autostereoscopic projection system, a smart technology enabled event, a smart technology enabled fitness class, a-smart vehicle, an augmented reality headset, a virtual reality headset, a metaverse headset, a mixed reality device, a virtual reality device, an augmented reality device, a game environment, a haptic glove, a haptic garment, a haptic footwear, a simulated environment, a virtual reality environment, an augmented reality environment, a mixed reality environment, a metaverse environment.

In some embodiments, there is provided a computer implemented method for providing a user device with an individualized wellness pattern aware intervention for a specific type of multi-feel state longitudinal journey over a time duration. The method involves: transmitting the individualized wellness pattern aware intervention using a communication interface; generating the individualized wellness pattern aware intervention using hardware processor programmed with executable instructions by: receiving individualization input data associated with a user; generating a data object using the input data; determining one or more intervention types to generate based on the data object by applying a plurality of computer models to the data object; generating one or more individualized wellness pattern aware intervention using the intervention types; performing measurements using one or more input devices to for at least part of the input data; and receiving the intervention at user device and activating, triggering or presenting the intervention.

In some embodiments, the processor generates the individualization data object by processing additional input data identifying qualities associated with the intervention from a simulation environment with one or more digital twins corresponding to one or more physical objects relating to the intervention.

In some embodiments, the plurality of computer models comprise a wellness pattern model, an individualization model, and an intervention model.

In some embodiments, the individualization input data comprises data pertaining to one or more of user specified preference, user specified goal, user specific wellness objective, user specified training plan, user skill baseline, user wellness baseline, user personality baseline, user activity baseline, identifying a potential user cohort, identifying a potential user accountability partner, user habit, user activities, contextual data about user habit, contextual data about user activity, user fitness class history, user purchase history, user community participation, user exercise logs, user biometric records, user social media activity, user online activity.

In some embodiments, there is provided a non-transitory computer readable medium with instructions stored thereon, that when executed by a hardware processor cause the processor to generate output data indicating an individualized wellness pattern aware intervention, wherein the processor generates the intervention by applying multiple computer models to an individualization data object generated by processing input data identifying qualities associated with the intervention. The instructions configure the hardware processor to trigger measurements by one or more sensors to receive the input data relating to physical physiological modeling. The processor generates the individualization data object by processing additional input data identifying qualities associated with the intervention from a simulation environment with one or more digital twins corresponding to one or more physical objects relating to the intervention. Additional input data comprises data relating to at least one of mood and emotion within the simulation environment.

In some embodiments, the individualized wellness pattern aware intervention comprises a multi-feel state longitudinal journey over a multi-day time duration to create a specific type of user experience over the multi-day time duration.

In some embodiments, the individualized wellness pattern aware intervention comprises an intervention output that is associated with an intervention type.

In some embodiments, the intervention type comprises one or more of suggesting an activity, starting an activity, suggesting a change in a current activity, changing a current activity, suggesting a change in the intensity of an activity, changing the intensity of an activity, canceling a scheduled activity, suggesting canceling a scheduled activity, suggesting the end of a current activity, ending a current activity, providing a reward, increasing a challenge, suggesting a product, providing a product, providing points, suggesting a future activity, starting a future activity, displaying a tool for user reflection, suggesting user feedback, displaying a tool for user feedback, suggesting a user chat or conversational activity, opening a user chat, suggesting an accountability partner, providing an accountability partner, suggesting a change in a current accountability partner, changing a current accountability partner, suggesting a team, providing a team, suggesting a change in a current team, changing a current team, suggesting a community, providing a community, suggesting a change in a current community, changing a current community, providing a level, removing a level, customizing a level, upgrading a level, downgrading a level, providing access to a product, providing access to an event, providing access to an experience, providing a badge, changing a badge, changing a social ranking, changing an indication of social belonging, changing an indication of contribution to a group, team or community, changing a social network connection, changing a group membership, providing a social connection, providing a means of communicating with another user, providing a means of sharing an emoticon with another user, providing feedback or an indication related to at least one of an activity being started, an activity in progress, an activity successfully completed, an activity duration, a user activity measure, a user activity success measure, a user activity partial success measure, a user activity in the context of a user's previous activity, a user activity in the context of a community activity, a user activity in the context of a digital twin data model, a user activity in the context of an ideal self data model, a user activity in the context of a cohort activity, a user activity in the context of a hero activity, a user activity in the context of a group of fitness class participants, a user activity in the context of an accountability partner, a user activity in the context of team, a user activity in the context of a community, a user's readiness to advance in an activity, a user's readiness to add a new activity, a user's readiness to stop an activity, a user's readiness to disrupt an activity, providing feedback or an indication related to at least one of a user habit being started, a user habit in progress, a user habit successfully completed, a user habit duration, a user habit activity measure, a user habit success measure, a user habit partial success measure, a user habit streak, a suggestion for resetting a habit, a suggestion for rescheduling a habit, rescheduling an activity, scheduling an activity, canceling a scheduled activity, a user's readiness to expand a habit, a user's readiness to add a new habit, a user's need to disrupt a habit, a user habit in the context of a community habit, a user habit in the context of a digital twin data model, a user habit in the context of an ideal self data model, a user habit in the context of a cohort habit, a user habit in the context of a hero habit, a user habit in the context of a group of fitness class participants, providing a virtual assistant, customizing a virtual assistant, providing information about a product, providing personalized information about a product, providing customized information about a product, providing a navigational path, providing a customized navigational path, providing a category of products, providing a customized category of products, and filtering a set of products, providing a special offer.

In some embodiments, the processor provides one or more media of the type video, interactive presentation, game, image, hologram image projection, autostereoscopic image projection, audio, text, spoken word, guided conversation, music, interactive simulation wherein the media contains content with a greater than average statistical probability to result in one or more of a technique correction, an emotional shift, an intellectual reframing of an experience, an emotional reframing of an experience, a distraction from a current or past experience, changing the user's anxiety level, changing the user's fear level, changing the user's hopefulness level, changing the user's sense of competence, changing the user's curiosity level, changing the user's feeling of agency, offering a relatable motivational experience, changing the user's competitiveness level, changing the user's cooperation level, changing the user's perception of social connection, changing the user's perception of personal advancement, changing the user's perception of social status, changing the user's perception of social belonging, changing the user's gratitude level, changing the user's calmness level, changing the user's focus level, changing the user's equanimity level, changing the user's sense of inner peace level.

In some embodiments, the multi-feel state longitudinal journey designed to create a specific type of user experience over a multi-day time duration is associated with one or more of the archetypal pattern wherein the archetypal pattern is one of, a quest for identity, a quest to find a ideal location, emotional state, or sense of spiritual realization, a quest for justice, a quest to help a community member, a quest for social connection, a quest for social acceptance within a group, a quest for a role or status designation, a quest in search of knowledge, competence or skill, a quest for acceptance and personal affirmation, a quest for transformation, a quest for self-actualization, a quest for pleasure, fun, surprise and adventure, a quest to remove a danger, a quest for a symbolic or metaphoric goal.

In some embodiments, the multi-feel state longitudinal journey designed to create a specific type of user experience over a multi-day time duration is associated with at least one of a pattern for adherence to a wellness, fitness lifestyle, basic health criteria, training plan, nutritional plan, and interventions are provided that are consistent with generating and improving the individual's adherence to the overall plan.

In some embodiments, the individualized wellness pattern aware intervention contains an interactive element that enable a user to engage with and/or select from more than one individualized wellness pattern aware intervention.

In some embodiments, the individualized wellness pattern aware intervention, is generated as a module with executable instructions to receive inputs, provide outputs and display the habit aware intervention.

In some embodiments, there is provided computer system for providing a user device with an individualized wellness pattern aware intervention for a specific type of multi-feel state longitudinal journey over a time duration. The system comprises a communication interface to transmit the individualized wellness pattern aware intervention; one or more non-transitory memory storing a trained individualization model, a trained wellness pattern model, and a trained intervention model; a hardware processor programmed with executable instructions for generating the individualized wellness pattern aware intervention. The hardware processor: receives input data; generates a data object using the input data; applies a wellness pattern model, an individualization model, and an intervention model to the data object; determines one or more intervention types to generate based on the data object, and values for the wellness pattern model, the individualization model and the intervention model; generates one or more individualized wellness pattern aware intervention using the intervention types. The system has one or more input devices to perform measurements for the input data, and a user device comprising a hardware processor, and an interface to receive the intervention, and activate, trigger or present the intervention. The hardware processor triggers measurements by one or more sensors to receive the input data relating to physical physiological modeling. The processor generates the individualization data object by processing additional input data identifying qualities associated with the intervention from a simulation environment with one or more digital twins corresponding to one or more physical objects relating to the intervention. Additional input data comprises data relating to at least one of mood and emotion within the simulation environment.

In some embodiments, the user device provides a selectable indicia to accept the individualized wellness pattern aware intervention.

In some embodiments, the individualized wellness pattern aware intervention is activated, triggered or presented automatically at the user device.

In some embodiments, the individualization input includes data pertaining to one or more of user specified preference, user specified goal, user specific wellness objective, user specified training plan, user skill baseline, user wellness baseline, user personality baseline, user activity baseline, identifying a potential user cohort, identifying a potential user accountability partner, user habit, user activities, contextual data about user habit, contextual data about user activity, user fitness class history, user purchase history, user community participation, user exercise logs, user biometric records, user social media activity, user online activity.

In some embodiments, the one or more input devices comprise hardware components for receiving one or more of text entry, physiological metrics, biometric identifiers, video, audio, conversation, image, Global Positioning System (GPS) location, camera input, body mapping with sensors, mapping user position in a physical space, eye tracking, IoT (Internet of Things) input, web browsing history, social media history, purchase history, chat history, EEG signals, Heart Rate HR, Heart Rate Variability HRV, respiratory rate, blood glucose, oximetry rates, electrodermal activity, weight, body mass index BMI values, body temperature, pH levels.

In some embodiments, the one or more input devices further comprises one or more of a biometric sensor, physiological sensor, a room sensor, microphone, still camera, video camera, body-based sensor, smart mat based sensor, smart weight based, smart bike based sensor, smart glove based sensor, garment based sensor, smart footwear based sensor, augmented reality headset, virtual reality headset, metaverse headset, mixed reality device, virtual reality device, an augmented reality device, photodetector sensors, sweat sensors, and dehydrations sensors.

In some embodiments, the communication interface transmits to an output devices comprising one or more of a web application, an application installed on a user device, a smart mirror device, a connected music system, a connected lighting system, a connected exercise mat, a connected heating device, a connected cooling device, a connected smell diffuser device, a connected electrical stimulation device, a connected implanted medical device, a virtual reality headset, an augmented reality headset, a metaverse headset, a mixed reality device, a virtual reality devices, an augmented reality device, a haptic glove, a game controller, a haptic garment, a retail application, a coaching application, a fitness class or studio application, a meditation application, a retail application, a meal or nutritional supplement delivery service application, an email system application, a text message system application, notification system application, augmented reality environment, simulated reality environment, virtual reality environment, mixed reality environment, a game environment, a metaverse environment.

In some embodiments, executable instructions configure the hardware processor to present, remove, or customize one or more of a retail offer, a retail experience, a user profile, user Wishlist, a workshop, coaching session, lecture, performance event, community event, an exercise class, an avatar, an avatar's apparel, a conversational interaction, a notification, a pop-up suggestion, an alarm, a badge, a reward, a number of points, a group membership.

In some embodiments, the system has a data storage device with a repository of previously generated individualized wellness pattern aware interventions.

In some embodiments, the processor evaluates a previously generated individualized wellness pattern aware intervention against one or more of context data, user data, community data, device capacity data, user preferences, connected device capacity data, availability of a community member data, availability of a cohort member data, availability of an accountability partner data, availability of team data and determine whether to regenerate, personalize, or augment the habit-aware intervention.

In some embodiments, the system provides an intervention within one or more of a fitness class environment, a studio environment, a class environment, a group activity environment, a performance environment, game environment, virtual reality system, simulated reality system, augmented reality system, mixed reality system, or metaverse system.

In some embodiments, the interface for presenting the intervention is one or more of a smart phone application, a web based application, computer based application, a smart mirror device, a smart watch, a item of smart jewelry, a item of smart apparel, a smart exercise bike, a smart gym, a smart weight, a smart lighting system, a smart audio system, a tablet, a computer, a device notification, a connected device such as a yoga mat, watch, heart rate monitor, breathing monitor, a blood glucose monitor, a galvanic skin response (GSR) monitor, an electronic implant, an EEG, a brain-computer interface. a hologram projection system, an autostereoscopic projection system, a smart technology enabled event, a smart technology enabled fitness class, a-smart vehicle, an augmented reality headset, a virtual reality headset, a metaverse headset, a mixed reality device, a virtual reality device, an augmented reality device, a game environment, a haptic glove, a haptic garment, a haptic footwear, a simulated environment, a virtual reality environment, an augmented reality environment, a mixed reality environment, a metaverse environment.

In some embodiments, a computer system generates an individualized wellness pattern aware intervention for a specific type of multi-feel state longitudinal journey over a time duration. The system has one or more non-transitory memory storing a trained individualization model, a trained wellness pattern model, and a trained intervention model; a hardware processor programmed with executable instructions in non-transitory memory to perform measurements using one or more data capture devices, and receive input data from the measurements associated with a cohort, a digital twin, an activity, a user context, user device intervention capacity, an individualization model, a wellness model, an intervention model, generate representational data objects, determine intervention types that match the generated representation values, map the representation values to the intervention type, and generate instructions for at least one wellness pattern aware intervention. The hardware processor is programmed with the executable instructions in the non-transitory memory to process the input data, evaluate at least one model of the trained individualization model, the trained wellness pattern model, and the trained intervention model, evaluate an activity, evaluate the intervention, evaluate a modification to the intervention, and generate the individualized wellness pattern aware intervention.

In some embodiments, the system has a non-transitory memory storing an intervention repository containing previously generated intervention instructions.

In some embodiments, the hardware processor is programmed with executable instructions to evaluate a previously generated intervention instructions and partially or completely regenerate interventions instructions.

In some embodiments, the individualization model comprises a personality component which measures at least three of openness, consciousness, extraversion, agreeableness, neuroticism, dominance, influence, steadiness, conscientiousness, inducement, introversion, sensing, intuition, thinking, feeling, judging, perceiving, driver, expressive, amiable, and analytical.

In some embodiments, the wellness pattern model comprises a wellness measure which calculates a value for at least two of positive emotion, engagement, positive relationships, meaning, achievements, health, competence, self-acceptance, social status, social belonging, mood, relatedness, resilience, giving, vitality, optimism, life satisfaction, reduction of negative emotions, increase of positive emotions, autonomy, purpose in life, environmental mastery, personal growth, purpose in life, pleasure, positive affect, emotional stability, social contribution, social integration, social acceptance, and social coherence.

In some embodiments, one or more of the trained individualization model, trained wellness pattern model, trained intervention model stored in the memory is updated by the hardware processor based on machine learning.

In some embodiments, the machine learning is based on one or more of cohort intervention feedback, user intervention feedback, user accountability partner intervention feedback, simulated user accountability partner intervention feedback, simulated cohort intervention feedback, simulated digital twin intervention feedback.

In some embodiments, the system has a generative adversarial network (GAN) evaluating one or more of intervention perceived ethics, enjoyability, conformance to social expectations, capacity to make a social connection, capacity to provide social recognition, capacity to support a specified goal, originality.

In some embodiments, the intervention model comprises an activity measure comprising a least 2 of a user preference for an activity, evaluation of activity effect using a cohort model, evaluation of activity effect using a digital twin model, activity start time, activity frequency, activity trigger or prior action, activity association with a habit, activity duration, activity measure, activity success measure, activity partial success measure, one or more methods of recognizing an activity completion, activity streak calculation, activity hedonic adaptation calculation, activity eudaimonic adaptation, user preference to discontinue a habit, user preference to discontinue an activity, identification of the association between an activity and an unwanted habit, identification of the association between an activity and an unwanted activity, readiness to expand activity calculation, readiness to add a new activity calculation, mapping of related habits, user reflection associated with an activity, user preference associated with the disruption of an activity.

In some embodiments, the activity measure comprises a planned, previous, predicted, or proposed activity value.

In some embodiments, there is provided a computer implemented method for generating an individualized wellness pattern aware intervention for a multi-state longitudinal journey over a time duration. The method involves: computing, using a hardware processor, representation values associated with a multi feel-state longitudinal journey designed to create a specific type of user experience over a multi-day time duration: performing measurements identifying qualities associated with the user, qualities associated with the user's current activity, and qualities associated with the user's current context; receiving input data from the measurements identifying qualities associated with the user, qualities associated with the user's current activity, and qualities associated with the user's current context; processing the input data to calculate an individualization data object, receiving additional input data identifying qualities associated with an intervention, determining one or more intervention types associated with the data object representation based on a wellness model, evaluating the intervention types based on the individualization data object, generating, using the hardware processor, instructions for an individualized intervention.

In some embodiments, the method involves receiving data representing one or more of a cohort of users, a simulated cohort of users, a simulated digital twin, a simulated best self, a simulated hero.

In some embodiments, the method involves receiving input data comprises receiving data from a simulation environment with one or more digital twins corresponding to one or more physical objects relating to the intervention.

In some embodiments, the receiving input data comprises receiving structured data, unstructured data, geographic location data, metadata, text, numeric values, images, renderings based on images, video, audio, sensor data, biometric data, physiological data, measurements of physical objects, timing data, time of day data, duration data, schedule data, genetic data, nutritional data, health kit data, recovery rating data, and stress scores data.

In some embodiments, the multi-feel state longitudinal journey designed to create a specific type of user experience over a multi-day time duration is associated with one or more of the archetypal pattern wherein the archetypal pattern is one of, a quest for identity, a quest to find a ideal location, emotional state, or sense of spiritual realization, a quest for justice, a quest to help a community member, a quest for social connection, a quest for social acceptance within a group, a quest for a role or status designation, a quest in search of knowledge, competence or skill, a quest for acceptance and personal affirmation, a quest for transformation, a quest for self-actualization, a quest for pleasure, fun, surprise and adventure, a quest to remove a danger, a quest for a symbolic or metaphoric goal.

In some embodiments, the multi-feel state longitudinal journey designed to create a specific type of user experience over a multi-day time duration is associated with at least one of a pattern for adherence to a wellness, fitness lifestyle, basic health criteria, training plan, nutritional plan, and interventions are provided that are consistent with generating and improving the individual's adherence to the overall plan

In some embodiments, the instructions for an individualized intervention is associated with an intervention type.

In some embodiments, the intervention type comprises one or more of suggesting an activity, starting an activity, suggesting a change in a current activity, changing a current activity, suggesting a change in the intensity of an activity, changing the intensity of an activity, canceling a scheduled activity, suggesting canceling a scheduled activity, suggesting the end of a current activity, ending a current activity, providing a reward, increasing a challenge, providing points, suggesting a product, providing a product, suggesting an accountability partner, providing an accountability partner, suggesting a change in a current accountability partner, changing a current accountability partner, suggesting a team, providing a team, suggesting a change in a current team, changing a current team, suggesting a community, providing a community, suggesting a change in a current community, changing a current community, providing a level, removing a level, customizing a level, upgrading a level, downgrading a level, providing access to a product, providing access to an event, providing access to an experience, providing a badge, changing a badge, changing a social ranking, changing an indication of social belonging, changing an indication of contribution to a group, team or community, changing a social network connection, changing a group membership, providing a social connection, providing a means of communicating with another user, providing a means of sharing an emoticon with another user, suggesting a future activity, starting a future activity, displaying a tool for user reflection, suggesting user provides feedback, displaying a tool for user feedback, suggesting a user chat or conversational activity, opening a user chat, providing feedback or an indication related to at least one of an activity being started, an activity in progress, an activity successfully completed, an activity duration, a user activity measure, a user activity success measure, a user activity partial success measure, a user activity in the context of a user's previous activity, a user activity in the context of a community activity, a user activity in the context of a digital twin data model, a user activity in the context of an ideal self data model, a user activity in the context of a cohort activity, a user activity in the context of a hero activity, a user activity in the context of a group of fitness class participants, a user activity in the context of an accountability partner, a user activity in the context of team, a user activity in the context of a community. a user's readiness to advance in an activity, a user's readiness to add a new activity, a user's readiness to stop an activity, a user's readiness to disrupt an activity, providing feedback or an indication related to at least one of a user habit being started, a user habit in progress, a user habit successfully completed, a user habit duration, a user habit activity measure, a user habit success measure, a user habit partial success measure, a user habit streak, a suggestion for resetting a habit, a suggestion for rescheduling a habit, rescheduling an activity, scheduling an activity, canceling a scheduled activity, a user's readiness to expand a habit, a user's readiness to add a new habit, a user's need to disrupt a habit, a user habit in the context of a community habit, a user habit in the context of a digital twin data model, a user habit in the context of an ideal self data model, a user habit in the context of a cohort habit, a user habit in the context of a hero habit, a user habit in the context of a group of fitness class participants, providing a virtual assistant, customizing a virtual assistant, providing information about a product, providing personalized information about a product, providing customized information about a product, providing a navigational path, providing a customized navigational path, providing a category of products, providing a customized category of products, filtering a set of products, and providing a special offer.

In some embodiments, the intervention type is associated with one or more options for providing the intervention type.

In some embodiments, the one or more option associated with the intervention type is based on one or more of user context, user device availability, user device capacity.

In some embodiments, the method involves generating, using the hardware processor, instructions for an individualized intervention further comprises providing one or more media of the type video, interactive presentation, game, image, hologram image projection, autostereoscopic image projection, audio, text, spoken word, guided conversation, music, interactive simulation wherein the media contains content with a greater than average statistical probability to result in one or more of a technique correction, an emotional shift, an intellectual reframing of an experience, an emotional reframing of an experience, a distraction from a current or past experience, changing the user's anxiety level, changing the user's fear level, changing the user's hopefulness level, changing the user's sense of competence, changing the user's curiosity level, changing the user's feeling of agency, offering a relatable motivational experience, changing the user's competitiveness level, changing the user's cooperation level, changing the user's perception of social connection, changing the user's perception of personal advancement, changing the user's perception of social status, changing the user's perception of social belonging, changing the user's gratitude level, changing the user's calmness level, changing the user's focus level, changing the user's equanimity level, changing the user's sense of inner peace level.

In some embodiments, instructions for an individualized intervention comprises an interactive element that enable a user to engage with and/or select from more than one individualized wellness pattern aware intervention.

In some embodiments, instructions for an individualized intervention, is generated as a module with executable instructions to receive inputs, provide outputs and display the habit aware intervention.

In some embodiments, instructions for an individualized intervention provide the intervention in one or more of a fitness class environment, a studio environment, a class environment, a group activity environment, a performance environment, game environment, virtual reality system, simulated reality system, augmented reality system, mixed reality system, or metaverse system.

In some embodiments, the instructions for an individualized intervention provide the intervention to one or more of a smart phone application, a web based application, computer based application, a smart mirror device, a smart watch, a item of smart jewelry, a item of smart apparel, a smart exercise bike, a smart gym, a smart weight, a smart lighting system, a smart audio system, a tablet, a computer, a device notification, a connected device such as a yoga mat, watch, heart rate monitor, breathing monitor, a blood glucose monitor, a galvanic skin response (GSR) monitor, an electronic implant, an EEG, a brain-computer interface. a hologram projection system, an autostereoscopic projection system, a smart technology enabled event, a smart technology enabled fitness class, a-smart vehicle, an augmented reality headset, a virtual reality headset, a metaverse headset, a mixed reality device, a virtual reality device, an augmented reality device, a game environment, a haptic glove, a haptic garment, a haptic footwear, a simulated environment, a virtual reality environment, an augmented reality environment, a metaverse environment.

In some embodiments, there is provided a computer implemented method for providing to a user device an individualized wellness pattern aware intervention for a multi-state longitudinal journey over a time duration. The method involves: performing measurements; receiving user context data from the measurements; mapping, using a hardware processor, the user context data to wellness, individualization, and intervention models; retrieving relevant potential intervention types; generating potential intervention type options; evaluating the potential intervention types based on the wellness, individualization, and intervention models; evaluating the potential intervention type options based on the wellness, individualization, and intervention models; evaluating the potential intervention types based on the context data; valuating the intervention options based on the context data; evaluating the potential intervention types based on user device capacity; evaluating the intervention options based on user device capacity; providing the individualized wellness pattern aware intervention to a user device to activate, trigger or present the intervention.

In some embodiments, the individualized wellness pattern aware intervention comprises more than one intervention.

In some embodiments, the more than one intervention is provided to more than one type of output device simultaneously.

In some embodiments, the user has an option to accept the individualized wellness pattern aware intervention.

In some embodiments, the individualized wellness pattern aware intervention is applied automatically.

In some embodiments, the method involves receiving user context data comprises one or more of a biometric sensor, physiological sensor, a room sensor, microphone, still camera, video camera, body-based sensor, smart mat based sensor, smart weight based, smart bike based sensor, smart glove based sensor, garment based sensor, smart footwear based sensor, augmented reality headset, virtual reality headset, metaverse headset, a mixed reality device, a virtual reality device, an augmented reality device.

In some embodiments, the method involves providing the individualized wellness pattern aware intervention comprises instructions for one or more of a web application, an application installed on a user device, a smart mirror device, a connected music system, a connected lighting system, a connected exercise mat, a connected heating device, a connected cooling device, a connected smell diffuser device, a connected electrical stimulation device, a connected implanted medical device, a virtual reality headset, an augmented reality headset, a metaverse headset, a mixed reality device, a virtual reality device, an augmented reality device, a haptic glove, a game controller, a haptic garment, a retail application, a coaching application, a fitness class or studio application, a retail application, a meal or nutritional supplement delivery service, an email system, a text message system, notification system, augmented reality environment, simulated reality environment, virtual reality environment, a mixed reality environment, a game environment, a metaverse environment.

In some embodiments, the method involves providing the individualized wellness pattern aware intervention comprises presenting, removing, or customizing one or more of a retail offer, a retail experience, a user profile, user Wishlist, a workshop, coaching session, lecture, performance event, community event, an exercise class, an avatar, an avatar's apparel, an avatar's appearance, a conversational interaction, a notification, a pop-up suggestion, an alarm, a badge, a reward, user points, a group membership.

In some embodiments, instructions are stored in a repository, retrieve from a repository, evaluate, partially regenerate, fully regenerate the individualized wellness pattern aware intervention.

In some embodiments, the method involves using instructions to personalize an intervention with one or more of a user's name, a user's date of birth, a user's nickname, a user's username, a user's preferred color, a user's preferred color scheme, a user's preferred visual aesthetic, a user preferred music type, a user's preferred song, a user's preferred avatar, a user's preferred avatar apparel, a user's preferred avatar appearance, a user's preferred badges, a user's preferred reward, a user's preferred groups, a user's geographic location, a user's time of day, a user's time zone, a user's friends, a user's preferred coach, a user's preferred hero, a user's preferred celebrity, a user's preferred instructor, a user's preferred lighting, a user's preferred temperature, a user's preferred.

In some embodiments, the method involves using executable instructions to evaluate a previously generated individualized wellness pattern aware intervention against one or more of context data, user data, community data, device capacity data, availability of a community member data, availability of a cohort member data, availability of an accountability partner data, availability of team data, user preferences, connected device capacity data, and determine whether to regenerate, customize, or augment the habit-aware intervention.

In some embodiments, the method involves using instructions to provide an indicator of the relationship between the intervention and the multi-state longitudinal journey.

In an aspect, embodiments described herein facilitate providing interventions to a user within the larger context, or model, of a multi-feel state longitudinal journey over a time duration or period. A multi-feel state longitudinal journey may be structured to create a specific type of user experience over a multi-day time duration, for example. By providing interventions consistent with a multi-feel state longitudinal journey, the user is nudged and/or rewarded to engage in behaviors that are consistent with a longer duration wellness model. In one aspect of some embodiments, engaging in a behavior provides a replacement behavior for an unwanted behavior, or ‘bad’ habit. In some embodiments, the user is provided with interventions that present, display or deliver information (such as statistics, charts, graphs, analysis, icons, indicators, audio feedback, images, text, rankings, contribution indicators, rewards, points, badges, etc.) that change the user's motivation level, competitiveness level and the like and further enable the user to track and visualize their behaviour within the context of a longer duration multi-feel state longitudinal journey over a time duration.

In an embodiment, the individualized wellness pattern aware intervention comprises an intervention output that is associated with an intervention type. Example intervention types provide a suggestion, or alternative, to the user and allow the user to provide feedback or make a selection. Some example interventions make a change to the user activity, user environment, or open a different tool or environment without the user making a selection. Embodiments described herein generate different types of intervention outputs. Examples of types of intervention outputs include, instructions or code (executable by a hardware processor) for automating one or more of suggesting an activity, starting an activity, suggesting a change in a current activity, changing a current activity, suggesting a change in the intensity of an activity, changing the intensity of an activity, canceling a scheduled activity, suggesting canceling a scheduled activity, suggesting the end of a current activity, ending a current activity, providing a reward, suggesting a product, providing a product, suggesting a future activity, starting a future activity, displaying a tool for user reflection, suggesting user feedback, displaying a tool for user feedback, suggesting a user chat or conversational activity, opening a user chat, suggesting an accountability partner, providing an accountability partner, suggesting a change in a current accountability partner, changing a current accountability partner, suggesting a team, providing a team, suggesting a change in a current team, changing a current team, suggesting a community, providing a community, suggesting a change in a current community, changing a current community, providing a badge, changing a badge, changing a social ranking, changing an indication of social belonging, changing an indication of contribution to a group, team or community, changing a social network connection, changing a group membership, providing a social connection, providing a means of communicating with another user, providing a means of sharing an emoticon with another user, providing feedback or an indication related to at least one of an activity being started, an activity in progress, an activity successfully completed, an activity duration, a user activity measure, a user activity success measure, a user activity partial success measure, a user activity in the context of a user's previous activity, a user activity in the context of a community activity, a user activity in the context of a digital twin data model, a user activity in the context of an ideal self data model, a user activity in the context of a cohort activity, a user activity in the context of a hero activity, a user activity in the context of a group of fitness class participants, a user activity in the context of an accountability partner, a user activity in the context of team, a user activity in the context of a community. a user's readiness to advance in an activity, a user's readiness to add a new activity, a user's readiness to stop an activity, a user's readiness to disrupt an activity, providing feedback or an indication related to at least one of a user habit being started, a user habit in progress, a user habit successfully completed, a user habit duration, a user habit activity measure, a user habit success measure, a user habit partial success measure, a user habit streak, a suggestion for resetting a habit, a suggestion for rescheduling a habit, rescheduling an activity, scheduling an activity, canceling a scheduled activity, a user's readiness to expand a habit, a user's readiness to add a new habit, a user's need to disrupt a habit, a user habit in the context of a community habit, a user habit in the context of a digital twin data model, a user habit in the context of an ideal self data model, a user habit in the context of a cohort habit, a user habit in the context of a hero habit, a user habit in the context of a group of fitness class participants. In some embodiments, the term suggesting one or more operations by hardware devices, such as a visualization or prompt at a display device, or audio feedback at a speaker, for example.

An intervention may include suggesting a product. Suggesting a product may include suggesting a product associated with a user's current, planned, or completed activities. In one embodiment, when a user selects and/or purchases a product, an intervention associated with the purchase context may be proposed. Suggesting a product includes suggesting one or more of a class, fitness accessory, item of apparel or the like.

In an embodiment, the intervention comprises feedback or an indication to the user with regard to the user activity, habit, performance, engagement, and the like, and may relate to the user in the past, a community, a digital twin data model, an ideal self data model, a cohort, a hero, a group of fitness class participants. A community may be defined in a number of ways, based on physical location, activity profile (beginner runner, advanced yogi, etc.), retail purchases, activity history, family, friendship group, social media links, academic affiliation, professional affiliation, employment affiliation, shared belief, and the like. A cohort is defined, in an example embodiment, to model behavior and intervention for a group of users sharing one or more common type aspects. A digital twin is defined, in an embodiment, to model behavior and intervention for a specific type of user or a representation of a specific user. An ideal self is modeled based on a number of factors such as exercise, nutrition, sleep, rest to model a user's own potential capacity, skill, performance, and the like. A hero is an ideal that is selected or provided to a user, and the hero may be modeled on an actual individual such as an instructor, a celebrity, a coach, an athlete, or an influencer or may be an amalgamation of ideals to provided as an aspirational model. In an embodiment, the digital twin, ideal self, and/or a combination of the digital twin and ideal self are used to define and model an accountability partner and/or select one or more accountability partner for a user from a cohort, community, or other collection of users and/or de-personalized representations of users.

Interventions may comprise providing one or more media of different types, such as video, interactive presentation, game, image, hologram image projection, autostereoscopic image projection, audio, text, spoken word, guided conversation, music, interactive simulation wherein the media contains content with a greater than average statistical probability to result in one or more of a technique correction, an emotional shift, an intellectual reframing of an experience, an emotional reframing of an experience, a distraction from a current or past experience, changing the user's anxiety level, changing the user's fear level, changing the user's hopefulness level, changing the user's sense of competence, changing the user's curiosity level, changing the user's feeling of agency, offering a relatable motivational experience, changing the user's competitiveness level, changing the user's cooperation level, changing the user's perception of social connection, changing the user's perception of personal advancement, changing the user's perception of social status, changing the user's perception of social belonging, changing the user's gratitude level, changing the user's calmness level, changing the user's focus level, changing the user's equanimity level, changing the user's sense of inner peace level. For example, in an embodiment, specific video, motivational text, or color backgrounds are displayed at a display device behind an instructor; in an example embodiment a short fun reward video is displayed at a display device when the user achieves a new success level; in an embodiment engaging or anxiety inducing music is provided when a user's eye focus shifts from the instruction.

In an embodiment, the multi-feel state longitudinal journey designed to create a specific type of user experience over a multi-day time duration is associated with one or more of the archetypal pattern, wherein the archetypal pattern is one of, a quest for identity, a quest to find a ideal location, emotional state, or sense of spiritual realization, a quest for justice, a quest to help a community member, a quest in search of knowledge, competence or skill, a quest for acceptance and personal affirmation, a quest for transformation, a quest for self-actualization, a quest for pleasure, fun, surprise and adventure, a quest to remove a danger, a quest for social connection, a quest for social acceptance within a group, a quest for a role or status designation, a quest for a symbolic or metaphoric goal. In an embodiment, this archetypal pattern is implicit in the interventions provided and the user is not aware of the archetypal pattern informing the multi-feel state longitudinal journey; in an embodiment this archetypal pattern is explicit in the interventions provided and the user is aware of the archetypal pattern informing the multi-feel state longitudinal journey; in an embodiment, the archetypal pattern is at times provided implicitly and at times provided explicitly; in one embodiment, the user has an option to select an archetypal pattern; in an embodiment, the archetypal pattern is selected based on user metadata such as preferences, past activity, past responses to intervention, user goals, personality, and the like. User goals, preferences, and personality may be explicitly or implicitly determined. In an embodiment, the multi-feel state longitudinal journey designed to create a specific type of user experience over a multi-day time duration is associated with a pattern for adherence to a wellness, fitness lifestyle, basic health criteria, training plan, nutritional plan, and interventions are provided that are consistent with generating and improving the individual's adherence to the overall plan.

In an embodiment, the individualized wellness pattern aware intervention contains an interactive element that enable a user to engage with and/or select from more than one individualized wellness pattern aware intervention. In one embodiment, the individualized wellness pattern aware intervention, is generated as a module with executable instructions to receive inputs, provide outputs and display the habit aware intervention.

In an embodiment, a system is provided for providing a user with an individualized wellness pattern aware intervention for a specific type of multi-feel state longitudinal journey over a time duration. The system has an output device to provide the individualized wellness pattern aware intervention; the system has a non-transitory memory storing a trained individualization model, a non-transitory memory storing a trained wellness pattern model, and a non-transitory memory storing a trained intervention model. The system has a hardware processor programmed with executable instructions for generating the individualized wellness pattern aware intervention. The hardware processor: receives input data; generates a data object using the input data; applies a wellness pattern model, an individualization model, and an intervention model to the data object; determines one or more intervention types (e.g. a preferred, best, or optimal intervention type) to generate based on the data object, the wellness pattern model, the individualization model and the intervention model values; generates one or more individualized wellness pattern aware intervention. The system has a user device comprising a hardware processor, one or more input devices; and a user device comprising a hardware processor, and one or more devices for activating, triggering, or presenting the intervention.

In an embodiment, the user has an option to accept the individualized wellness pattern aware intervention. In an embodiment, the individualized wellness pattern aware intervention is applied automatically. In an embodiment, there is variation in whether the user has an option to accept the intervention, or the intervention is applied automatically. This variation may be based on the context, the intervention type, the user's history of accepting an intervention, the wellness pattern, user preferences, and the like.

In an embodiment, the system's individualization input includes data pertaining to one or more of user specified preference, user specified goal, user specific wellness objective, user specified training plan, user skill baseline, user wellness baseline, user personality baseline, user activity baseline, identifying a potential user cohort, user habit, user activities, contextual data about user habit, contextual data about user activity, user fitness class history, user purchase history, user community participation, user exercise logs, user biometric records.

The system receives data from one or more input devices that generate different types of input data, such as text entry, physiological metrics, biometric identifiers, video, audio, conversation, image, Global Positioning System (GPS) location, camera input, body mapping with sensors, mapping user position in a physical space, eye tracking, IoT (Internet of Things) input, web browsing history, social media history, purchase history, chat history, EEG signals, Heart Rate HR, Heart Rate Variability HRV, galvanic skin response (GSR), respiratory rate, blood glucose, oximetry rates, weight, body mass index BMI values, body temperature, pH levels.

In one embodiment, the system receives input data that includes data associated with a mood designation, designation indirectly associated with a mood, emotion, emotion intensity, valence measurement. A user may provide an input (through controls, text, voice, images, video, or the like) and this input may be analysed to extrapolate a mood, emotion, intensity of mood, intensity of emotion, or valence rating. This input may be associated with a specific intervention, intervention type, activity, activity type, user context, user context type. In some embodiments, the system can receive input data relating to non-physical physiological user attributes, and input data relating to physical physiological user attributes.

In an embodiment, the user device in the system further comprises one or more of a biometric sensor, physiological sensor, a room sensor, microphone, still camera, video camera, body-based sensor, smart mat based sensor, smart weight based, smart bike based sensor, smart glove based sensor, garment based sensor, smart footwear based sensor, augmented reality headset, virtual reality headset, metaverse headset, a mixed reality device, a virtual reality device, an augmented reality device. These sensors may be combined in a number of combinations as is evident in the sensor systems which are commercially available.

In an embodiment, the system output devices or output data can involve one or more of a web application, an application installed on a user device, a smart mirror device, a connected music system, a connected lighting system, a connected exercise mat, a connected heating device, a connected cooling device, a connected smell diffuser device, a connected electrical stimulation device, a connected implanted medical device, a virtual reality headset, an augmented reality headset, a metaverse headset, a mixed reality device, a virtual reality device, an augmented reality device, a haptic glove, a game controller, a haptic garment, a retail application, a coaching application, a fitness class or studio application, a retail application, a meal or nutritional supplement delivery service, an email system, a text message system, notification system, augmented reality environment, simulated reality environment, virtual reality environment, a mixed reality environment, a game environment, a metaverse environment. These output devices may be combined in a number of combinations. Independent output devices, and output devices within connected environments, augmented reality, virtual reality and integrated systems provide outputs in parallel, combination, and/or individually that may comprise the intervention.

In an embodiment, the system further comprises executable instructions to present, remove, or customize one or more of a retail offer, a retail experience, a user profile, user Wishlist, a workshop, coaching session, lecture, performance event, community event, an exercise class, an avatar, an avatar's apparel, an avatar's appearance, a conversational interaction, a notification, a pop-up suggestion, an alarm, a badge, a reward, a number of points, a group membership, and so on.

In an embodiment, the user device associated with the system provides a selectable indicia. This selectable indicia may be used to accept the individualized wellness pattern aware intervention. In some embodiments, the indicia may be used to modify, reject, or view alternatives to a proposed individualized wellness pattern aware intervention.

In an embodiment, the system further comprises a data storage device, such a server, database, or other device, with a repository of previously generated individualized wellness pattern aware interventions. In one aspect, this embodiment further comprises executable instructions to evaluate a previously generated individualized wellness pattern aware intervention against one or more of context data, user data, community data, device capacity data, user preferences, connected device capacity data, availability of a community member data, availability of a cohort member data, availability of an accountability partner data, availability of team data and determine whether to regenerate, personalize, or augment the habit-aware intervention. In some embodiments, regenerating an intervention regenerates a subset of the intervention instructions. Personalization can include augmenting the intervention with personalization such as the user's name, the username, age, gender, user's birthday, user's personal goals, user's hero, user's motivational texts, user's badges, user's status, avatar preferences, color preferences, music preferences, location, preferred studio, preferred store, income, family, pets, education level and the like.

In an embodiment, the system provides an intervention within one or more of a fitness class environment, a studio environment, a class environment, a group activity environment, a performance environment, game environment, virtual reality system, simulated reality system, augmented reality system, or metaverse system.

Is an embodiment, output device(s) may enable one or more of presenting the intervention is one or more of a smart phone application, a web based application, computer based application, a smart mirror device, a smart watch, a item of smart jewelry (such as a ring, necklace, bracelet, anklet, piercing jewelry, or the like) a item of smart apparel, a smart exercise bike, a smart gym, a smart weight, a smart lighting system, a smart audio system, a tablet, a computer, a device notification, a connected device such as a yoga mat, watch, heart rate monitor, breathing monitor, a blood glucose monitor, an electronic implant, an EEG, a brain-computer interface. a hologram projection system, an autostereoscopic projection system, a smart technology enabled event, a smart technology enabled fitness class, a-smart vehicle, an augmented reality headset, a virtual reality headset, a mixed reality device, a virtual reality device, an augmented reality device, a metaverse headset, a game environment, a haptic glove, a haptic garment, a haptic footwear, a simulated environment, a virtual reality environment, an augmented reality environment, a metaverse environment. These devices or components may be combined. An intervention may be provided by a number of methods in combination. The intervention may be provided in parallel, combination, and/or individually on one or more devices or components for activating or presenting the intervention.

In another aspect, there is provided a system for generating an individualized wellness pattern aware intervention to facilitate a specific type of multi-feel state longitudinal journey. The system has: a non-transitory memory storing a trained individualization model, a non-transitory memory storing a trained wellness pattern model; a non-transitory memory storing a trained intervention model; a hardware processor programmed with executable instructions in non-transitory memory to receive data input associated with a cohort, a digital twin, an activity, a user context, user device intervention capacity, an individualization model, a wellness model, an intervention model, generate representational data objects, determine intervention types that match the generated representation values, map the representation values to the intervention type, and generate instructions for at least one wellness pattern aware intervention; a hardware processor programmed with executable instructions in non-transitory memory to evaluate a model, evaluate an activity, evaluate an intervention, evaluate a modification to an intervention.

In an aspect of the embodiment, the system further comprises a non-transitory memory storing an intervention repository containing previously generated intervention instructions. In one embodiment, this repository further comprises executable instructions to evaluate a previously generated intervention instructions and partially or completely regenerate interventions instructions.

In an embodiment, the individualization model comprises a personality component which measures at least three personality factors that measure or estimate different aspects of personality. Example personality factors include openness, consciousness, extraversion, agreeableness, neuroticism, dominance, influence, steadiness, conscientiousness, inducement, introversion, sensing, intuition, thinking, feeling, judging, perceiving, driver, expressive, amiable, and analytical.

In an embodiment, the individualization model comprises a personality component which measures motivation associated with an individual based on a competitiveness scale and a player orientation to self or others scale, and/or a combination of competitiveness and self-other orientation. In an embodiment, the activity model determines a motivation associated with a user, digital twin, accountability partner, user cohort and/or a motivation associated with an activity, activity type, activity category, series of activities, and/or a combination.

In an embodiment, the wellness pattern model comprises a wellness measure which calculates a value for at least two of positive emotion, engagement, positive relationships, meaning, achievements, health, competence, self-acceptance, social status, social belonging, mood, relatedness, resilience, giving, vitality, optimism, life satisfaction, reduction of negative emotions, increase of positive emotions, autonomy, purpose in life, environmental mastery, personal growth, purpose in life, pleasure, positive affect, emotional stability, social contribution, social integration, social acceptance, and social coherence.

In an embodiment, the trained individualization model, trained wellness pattern model, trained intervention model is updated using feedback data based on machine learning. In one embodiment of this embodiment, the machine learning is based on one or more of cohort intervention feedback, user intervention feedback, user accountability partner intervention feedback, simulated user accountability partner intervention feedback, simulated cohort intervention feedback, simulated digital twin intervention feedback.

In an embodiment, the system includes a generative adversarial network (GAN) evaluating one or more of intervention perceived ethics, enjoyability, conformance to social expectations, capacity to make a social connection, capacity to provide social recognition, capacity to support a specified goal, originality.

In another aspect, there is provided a computer implemented method for generating an individualized wellness pattern aware intervention to facilitate a multi-state longitudinal journey, the method comprising: computing, using a hardware processor, representation values associated with a multi feel-state longitudinal journey designed to create a specific type of user experience over a multi-day time duration: receiving input data identifying qualities associated with the user; receiving input data identifying qualities associated with the user's current activity; receiving input data identifying qualities associated with the user's current context; processing input data to calculate an individualization data object, receiving input data identifying qualities associated with an intervention, determining one or more intervention type associated with the data object representation based on a wellness model, evaluating the intervention types based on the individualization data object, generating, using the hardware processor, instructions for an individualized intervention.

In an embodiment, the multi-state longitudinal journey factors one or more extrinsic motivation adaptation, intrinsic motivation adaptation, hedonic adaptation, eudaimonic adaptation, resilience adaptation, external reward entitlement adaptation, adaptation to elements that previously offered surprise or novelty. In an embodiment, the multi-state longitudinal journey provides opportunities for progression such as leveling up, new challenges, access to new cohorts, unlocking levels of activity, changing status within a community, and the like. In an embodiment, the underlying logic of the multi-state longitudinal journey supports a transition from a primarily extrinsic motivation to a primarily intrinsic motivation. In an embodiment the multi-state longitudinal journey supports a transition from lower user effort leading to small rewards to greater user effort leading to larger rewards. In an embodiment the multi-state longitudinal journey supports a greater variety of user effort types and/or behaviours leading to a reward and/or a greater variety of reward types.

In an embodiment, the computer implemented method for generating an individualized wellness pattern aware intervention includes receiving data representing one or more of a cohort of users, a simulated cohort of users, a simulated digital twin, a simulated best self, a simulated accountability partner, a simulated hero. This receiving input data may further comprise receiving data from a simulation. Receiving input data may include receiving structured data, unstructured data, geographic location data, metadata, text, numeric values, images, renderings based on images, video, audio, sensor data, biometric data, physiological data, measurements of physical objects, timing data, time of day data, duration data, schedule data, genetic data, nutritional data, health kit data, recovery rating data, and stress scores data.

In an embodiment, the intervention model comprises an activity measure comprising a least two of a user preference for an activity, evaluation of activity effect using a cohort model, evaluation of activity effect using a digital twin model, activity start time, activity frequency, activity trigger or prior action, activity association with a habit, activity duration, activity measure, activity success measure, activity partial success measure, one or more methods of recognizing an activity completion, activity streak calculation, activity hedonic adaptation calculation, activity eudaimonic adaptation, user preference to discontinue a habit, user preference to discontinue an activity, identification of the association between an activity and an unwanted habit, identification of the association between an activity and an unwanted activity, readiness to expand activity calculation, readiness to add a new activity calculation, mapping of related habits, user reflection associated with an activity, user preference associated with the disruption of an activity. In one embodiment, the activity measure comprises a planned, previous, predicted, or proposed activity value.

In an embodiment, the computer implemented method for generating an individualized wellness pattern aware intervention includes a multi-feel state longitudinal journey model designed evaluate an intervention's capacity within a specific type of user experience over a multi-day time duration where the multi-feel state longitudinal journey model is associated with one or more of the archetypal pattern wherein the archetypal pattern is one of, a quest for identity, a quest to find a ideal location, emotional state, or sense of spiritual realization, a quest for justice, a quest to help a cohort, a quest for social connection, a quest for social acceptance within a group, a quest for a role or status designation, a quest in search of knowledge, competence or skill, a quest for acceptance and personal affirmation, a quest for transformation, a quest for self-actualization, a quest for pleasure, fun, surprise and adventure, a quest to remove a danger, a quest for a symbolic or metaphoric goal.

In an embodiment, the computer implemented method for generating an individualized wellness pattern aware intervention includes instructions to associate an individualized intervention with an intervention type. In one embodiment, intervention type involves one or more of suggesting an activity, starting an activity, suggesting a change in a current activity, changing a current activity, suggesting a change in the intensity of an activity, changing the intensity of an activity, canceling a scheduled activity, suggesting canceling a scheduled activity, suggesting the end of a current activity, ending a current activity, providing a reward, suggesting a product, providing a product, suggesting a future activity, starting a future activity, displaying a tool for user reflection, suggesting user feedback, displaying a tool for user feedback, suggesting a user chat or conversational activity, opening a user chat, suggesting an accountability partner, providing an accountability partner, suggesting a change in a current accountability partner, changing a current accountability partner, suggesting a team, providing a team, suggesting a change in a current team, changing a current team, suggesting a community, providing a community, suggesting a change in a current community, changing a current community, providing a badge, changing a badge, changing a social ranking, changing an indication of social belonging, changing an indication of contribution to a group, team or community, changing a social network connection, changing a group membership, providing a social connection, providing a means of communicating with another user, providing a means of sharing an emoticon with another user, providing feedback or an indication related to at least one of an activity being started, an activity in progress, an activity successfully completed, an activity duration, a user activity measure, a user activity success measure, a user activity partial success measure, a user activity in the context of a user's previous activity, a user activity in the context of a community activity, a user activity in the context of a digital twin data model, a user activity in the context of an ideal self data model, a user activity in the context of a cohort activity, a user activity in the context of a hero activity, a user activity in the context of a group of fitness class participants, a user activity in the context of an accountability partner, a user activity in the context of team, a user activity in the context of a community. a user's readiness to advance in an activity, a user's readiness to add a new activity, a user's readiness to stop an activity, a user's readiness to disrupt an activity, providing feedback or an indication related to at least one of a user habit being started, a user habit in progress, a user habit successfully completed, a user habit duration, a user habit activity measure, a user habit success measure, a user habit partial success measure, a user habit streak, a suggestion for resetting a habit, a suggestion for rescheduling a habit, rescheduling an activity, scheduling an activity, canceling a scheduled activity, a user's readiness to expand a habit, a user's readiness to add a new habit, a user's need to disrupt a habit, a user habit in the context of a community habit, a user habit in the context of a digital twin data model, a user habit in the context of an ideal self data model, a user habit in the context of a cohort habit, a user habit in the context of a hero habit, a user habit in the context of a group of fitness class participants.

In an embodiment, the computer implemented method for generating an individualized wellness pattern aware intervention type is associated with one or more option for providing the intervention type. In one embodiment, the one or more option associated with the intervention type is based on one or more of user context, user device availability, user device capacity.

In an embodiment, generating, using the hardware processor, instructions for an individualized intervention further comprises providing one or more media of the type video, interactive presentation, game, image, hologram image projection, autostereoscopic image projection, audio, text, spoken word, guided conversation, music, interactive simulation wherein the media contains content with a greater than average statistical probability to result in one or more of a technique correction, an emotional shift, an intellectual reframing of an experience, an emotional reframing of an experience, a distraction from a current or past experience, changing the user's anxiety level, changing the user's fear level, changing the user's hopefulness level, changing the user's sense of competence, changing the user's curiosity level, changing the user's feeling of agency, offering a relatable motivational experience, changing the user's competitiveness level, changing the user's cooperation level, changing the user's perception of social connection, changing the user's perception of personal advancement, changing the user's perception of social status, changing the user's perception of social belonging, changing the user's gratitude level, changing the user's calmness level, changing the user's focus level, changing the user's equanimity level, changing the user's sense of inner peace level.

In an embodiment, instructions for an individualized intervention comprises an interactive element that enable a user to engage with and/or select from more than one individualized wellness pattern aware intervention. In one embodiment, instructions for an individualized intervention, is generated as a module with executable instructions to receive inputs, provide outputs and display the habit aware intervention.

In an embodiment, the instructions for an individualized intervention provide the intervention in one or more of a fitness class environment, a studio environment, a class environment, a group activity environment, a performance environment, game environment, virtual reality system, simulated reality system, augmented reality system, or metaverse system. In one embodiment, the instructions for an individualized intervention provide the intervention to one or more of a smart phone application, a web based application, computer based application, a smart mirror device, a smart watch, a item of smart jewelry, a item of smart apparel, a smart exercise bike, a smart gym, a smart weight, a smart lighting system, a smart audio system, a tablet, a computer, a device notification, a connected device such as a yoga mat, watch, heart rate monitor, breathing monitor, a blood glucose monitor, an electronic implant, an EEG, a brain-computer interface. a hologram projection system, an autostereoscopic projection system, a smart technology enabled event, a smart technology enabled fitness class, a-smart vehicle, an augmented reality headset, a virtual reality headset, a mixed reality device, a virtual reality device, an augmented reality device, a metaverse headset, a game environment, a haptic glove, a haptic garment, a haptic footwear, a simulated environment, a virtual reality environment, an augmented reality environment, a metaverse environment.

In another aspect, there is provided a computer implemented method for providing to a user an individualized wellness pattern aware intervention to facilitate a multi-state longitudinal journey comprising: receiving user context data; mapping, using a hardware processor, user context data to wellness, individualization, and intervention models; retrieving relevant potential intervention types; generating potential intervention type options; evaluating potential intervention types based on the wellness, individualization, and intervention models; evaluating potential intervention type options based on the wellness, individualization, and intervention models; evaluating potential intervention types based on context data; evaluating intervention options based on context data; evaluating potential intervention types based on user device capacity; evaluating intervention options based on user device capacity; providing the individualized wellness pattern aware intervention.

In an embodiment, the individualized wellness pattern aware intervention comprises more than one intervention. In one embodiment, the more than one intervention is provided to more than one type of output device simultaneously. In one embodiment, the user has an option to accept the individualized wellness pattern aware intervention. In one embodiment, the individualized wellness pattern aware intervention is applied automatically.

In an embodiment of the method for providing the individualized wellness pattern aware intervention, receiving user context data comprises one or more of a biometric sensor, physiological sensor, a room sensor, microphone, still camera, video camera, body-based sensor, smart mat based sensor, smart weight based, smart bike based sensor, smart glove based sensor, garment based sensor, smart footwear based sensor, augmented reality headset, virtual reality headset, metaverse headset, and a mixed reality device, a virtual reality device, an augmented reality device.

In an embodiment of the method for providing the individualized wellness pattern aware intervention, the providing the individualized wellness pattern aware intervention comprises instructions for one or more of a web application, an application installed on a user device, a smart mirror device, a connected music system, a connected lighting system, a connected exercise mat, a connected heating device, a connected cooling device, a connected smell diffuser device, a connected electrical stimulation device, a connected implanted medical device, a virtual reality headset, an augmented reality headset, a metaverse headset, a mixed reality device, a virtual reality device, an augmented reality device, a haptic glove, a game controller, a haptic garment, a retail application, a coaching application, a fitness class or studio application, a retail application, a meal or nutritional supplement delivery service, an email system, a text message system, notification system, augmented reality environment, simulated reality environment, virtual reality environment, a game environment, a metaverse environment.

In an embodiment of the method for providing the individualized wellness pattern aware intervention, the providing the individualized wellness pattern aware intervention comprises presenting, removing, or customizing one or more of a retail offer, a retail experience, a user profile, user Wishlist, a workshop, coaching session, lecture, performance event, community event, a exercise class, an avatar, an avatar's apparel, an avatar's appearance, a conversational interaction, a notification, a pop-up suggestion, a alarm, a badge, a reward, a number of points, a group membership.

An embodiment of the method for providing the individualized wellness pattern aware intervention, includes instructions to store in a repository, retrieve from a repository, evaluate, partially regenerate, fully regenerate the individualized wellness pattern aware intervention. In one embodiment of the method for providing the individualized wellness pattern aware intervention, includes instructions to personalize an interaction with one or more of a user's name, a user's date of birth, a user's nickname, a user's username, a user's preferred color, a user's preferred color scheme, a user's preferred visual aesthetic, a user preferred music type, a user's preferred song, a user's preferred avatar, a user's preferred avatar apparel, a user's preferred avatar appearance, a user's preferred badges, a user's preferred reward, a user's preferred groups, a user's geographic location, a user's time of day, a user's time zone, a user's friends, a user's preferred coach, a user's preferred hero, a user's preferred celebrity, a user's preferred instructor, a user's preferred lighting, a user's preferred temperature, a user's preferred season, schedule data, genetic data, nutritional data, health kit data, recovery ratings, stress scores. In one embodiment of the method for providing the individualized wellness pattern aware intervention, includes executable instructions to evaluate a previously generated individualized wellness pattern aware intervention against one or more of context data, user data, community data, device capacity data, availability of a community member data, availability of a cohort member data, availability of an accountability partner data, availability of team data, user preferences, connected device capacity data, and determine whether to regenerate, customize, or augment the habit-aware intervention.

In an embodiment of the method for providing the individualized wellness pattern aware intervention, includes instructions to provide an indicator of the relationship between the intervention and the multi-state longitudinal journey.

This summary does not necessarily describe the entire scope of all aspects. Other aspects, features and advantages will be apparent to those of ordinary skill in the art upon review of the following description of specific embodiments.

### BRIEF DESCRIPTION OF THE DRAWINGS

Embodiments of the disclosure will now be described in conjunction with the accompanying drawings of which:

FIG. 1 shows an example system architecture for generating and/or providing an individualized wellness pattern aware intervention based on a wellness model for users, according to embodiments described herein.

FIG. 2 shows an example system architecture for generating and/or providing an individualized wellness pattern aware intervention based on a wellness model for users, according to embodiments described herein.

FIG. 3 shows an example method of generating an individualized wellness pattern aware intervention based on a wellness model, according to embodiments described herein.

FIG. 4 shows an example method of generating an individualized wellness pattern aware intervention, according to embodiments described herein.

FIG. 5 shows an example method of generating and/or providing an individualized wellness pattern aware intervention, according to embodiments described herein.

FIG. 6 shows an example method of generating an individualized wellness pattern aware intervention, according to embodiments described herein.

FIG. 7 shows an aspect related to generating and/or providing an individualized wellness pattern aware intervention, according to embodiments described herein.

FIG. 8 shows an aspect related to generating and/or providing an individualized wellness pattern aware intervention, according to embodiments described herein.

FIG. 9 shows an aspect related to generating and/or providing an individualized wellness pattern aware intervention, according to embodiments described herein.

FIG. 10 shows an example method of generating an individualized wellness pattern aware intervention, according to embodiments described herein.

FIG. 11 shows an example method of providing an individualized wellness pattern aware intervention, according to embodiments described herein.

FIG. 12 shows an example user interface providing an individualized wellness pattern aware intervention, according to embodiments described herein.

FIG. 13 shows an example smart environment system providing one or more individualized wellness pattern aware intervention, according to embodiments described herein.

FIG. 14 shows an example individualized wellness pattern aware intervention embodiment, according to embodiments described herein.

FIG. 15 shows an example individualized wellness pattern aware intervention embodiment, according to embodiments described herein.

### DETAILED DESCRIPTION

Embodiments relate to methods and systems with non-transitory memory storing instructions and data records for wellness characterization, individualization characterization, intervention characterization. The methods and systems involve a hardware processor having executable instructions to provide one or more generated wellness pattern aware intervention instruction. These instructions are then transmitted to and interpreted by an application and/or output device to activate or trigger one or more intervention, or provide the user with one or more intervention. In an aspect, embodiments described herein provide, to the user, an individualized wellness pattern aware intervention, also referred to as an intervention.

Embodiments described herein can provide improved methods and systems for providing the user with an intervention, or activating or triggering one or more intervention. Embodiments described herein can involve automatically generating and transmitting control signals relating to intervention instructions to one or more hardware components used to provide the intervention to improve and control the computer hardware functionality. For example, control signals can be sent to a display device to control the display of visualizations relating to the intervention (e.g. simulating a digital twin and modifying the display of a visualization of the digital twin), or a speaker to control generation of audio output relating to the intervention.

Embodiments described herein provide an intervention to a user. As noted, providing an intervention can involve controlling hardware devices with control comments defining instructions for the intervention. The intervention aligns the user behaviors with a larger context, or model, which defines one or more multi-feel state longitudinal journeys over a time duration. The multi-feel state longitudinal journey is structured to create a specific type of user experience over a multi-day time duration. For example, the multi-feel state longitudinal journey is structured as a series or set of interventions over a time duration. By providing interventions that are consistent with the multi-feel state longitudinal journey, the user is nudged and/or rewarded to engage in behaviors that are consistent with a longer duration wellness model. As another example, in an embodiment, the multi-feel state longitudinal journey designed to create a specific type of user experience over a multi-day time duration is associated with a pattern for adherence to a wellness, fitness lifestyle, basic health criteria, training plan, nutritional plan. Interventions are provided that are consistent with generating and improving the individual's adherence to the overall plan.

Embodiments of the disclosure provide methods and systems for determining a type of intervention (e.g. an appropriate or optimal type of intervention), generating the intervention, and improving the system capacity to provide an intervention that is accepted by the user, supports the user in the multi-feel state longitudinal journey, and/or improves the user's overall sense of wellness.

Interventions are evaluated automatically by one or more hardware processors based on their capacity to maintain, change, or stop a user activity to support an overall wellness pattern. Example intervention types include suggesting an activity, starting an activity, suggesting a change in a current activity, changing a current activity, suggesting a change in the intensity of an activity, changing the intensity of an activity, canceling a scheduled activity, suggesting canceling a scheduled activity, suggesting the end of a current activity, ending a current activity, providing a reward, suggesting a product, providing a product, suggesting a future activity, starting a future activity, displaying a tool for user reflection, suggesting user provides feedback, displaying a tool for user feedback, suggesting a user chat or conversational activity, opening a user chat, and so on.

Further examples of intervention types include providing feedback or an indication (e.g. at a display device, speaker) related to at least one of an activity being started, an activity in progress, an activity successfully completed, an activity duration, a user activity measure, a user activity success measure, a user activity partial success measure, a user activity in the context of a user's previous activity, a user activity in the context of a community activity, a user activity in the context of a digital twin data model, a user activity in the context of an ideal self data model, a user activity in the context of a cohort activity, a user activity in the context of a hero activity, a user activity in the context of a group of fitness class participants, a user's readiness to advance in an activity, a user's readiness to add a new activity, a user's readiness to stop an activity, a user's readiness to disrupt an activity, providing feedback or an indication related to at least one of a user habit being started, a user habit in progress, a user habit successfully completed, a user habit duration, a user habit activity measure, a user habit success measure, a user habit partial success measure, a user habit streak, a suggestion for resetting a habit, a suggestion for rescheduling a habit, rescheduling an activity, scheduling an activity, canceling a scheduled activity, a user's readiness to expand a habit, a user's readiness to add a new habit, a user's need to disrupt a habit, a user habit in the context of a community habit, a user habit in the context of a digital twin data model, a user habit in the context of an ideal self data model, a user habit in the context of a cohort habit, a user habit in the context of a hero habit, a user habit in the context of a group of fitness class participants.

In an embodiment, the intervention includes one or more of providing one or more point, token, membership or badge which may be exchanged for or used by the user for a reward. The reward may be one or more of a physical object, an experience, a fitness class, a duration with a personal trainer or coach, a charitable donation, entry in a raffle, sweepstakes, lottery, or contest, additional functionality within a user application or retail platform, an upgrade to their tier, an upgrade to their service, early access to functionality within a user application or retail platform, early access to purchase one or more product, early access to a discount, access to apparel modification service (such as hemming or tailoring), access to an apparel exchange or reseller program or service, access to additional retail services (such as a personal shopper, preferred return/exchange policy, bespoke retail experiences, trunk or fashion shows), access to a premium version of a service, an employment, contract, or licensing offer, a promotion or cross-promotion of the user's online, social media, business, or other content, an event invitation, a discount on a physical object, set of physical objects, online retail cart or purchase value, in person retail cart or purchase value, an experience, a fitness class, a duration with a personal trainer or coach, additional functionality, an event, or the like.

Interventions may involve various media types and combinations of various media types. Example media types include video, interactive presentation, game, image, hologram image projection, autostereoscopic image projection, audio, text, spoken word, guided conversation, music, interactive simulation. The media may be selected as intervention, either independently or combined with other types of intervention, based on the content having a greater than average statistical probability to result in one or more of a technique correction, an emotional shift, an intellectual reframing of an experience, an emotional reframing of an experience, a distraction from a current or past experience, changing the user's anxiety level, changing the user's fear level, changing the user's hopefulness level, changing the user's sense of competence, changing the user's curiosity level, changing the user's feeling of agency, offering a relatable motivational experience, changing the user's competitiveness level, changing the user's cooperation level, changing the user's perception of social connection, changing the user's perception of personal advancement, changing the user's perception of social status, changing the user's perception of social belonging, changing the user's gratitude level, changing the user's calmness level, changing the user's focus level, changing the user's equanimity level, changing the user's sense of inner peace level.

In an embodiment, an intervention may further involve executable instructions to present, remove, or customize one or more of a retail offer, a retail experience, a user profile, user wish list of products or services, a workshop, coaching session, lecture, performance event, community event, an exercise class, an avatar, an avatar's apparel, an avatar's appearance, a conversational interaction, a notification, a pop-up suggestion, an alarm, a badge, a reward, a number of points, a group membership.

An intervention may be provided in different ways and using different devices, including, for example, one or more of a web application, an application installed on a user device, a smart mirror device, a connected music system, a connected lighting system, a connected exercise mat, a connected heating device, a connected cooling device, a connected smell diffuser device, a connected electrical stimulation device, a connected implanted medical device, a virtual reality headset, an augmented reality headset, a metaverse headset, a haptic glove, a game controller, a haptic garment, a retail application, a coaching application, a fitness class or studio application, a retail application, a meal or nutritional supplement delivery service, an email system, a text message system, notification system, augmented reality environment, simulated reality environment, virtual reality environment, a game environment, a metaverse or virtual environment. As noted, embodiments described herein can involve generating control commands to control operation of different types of devices used to provide the intervention for the user.

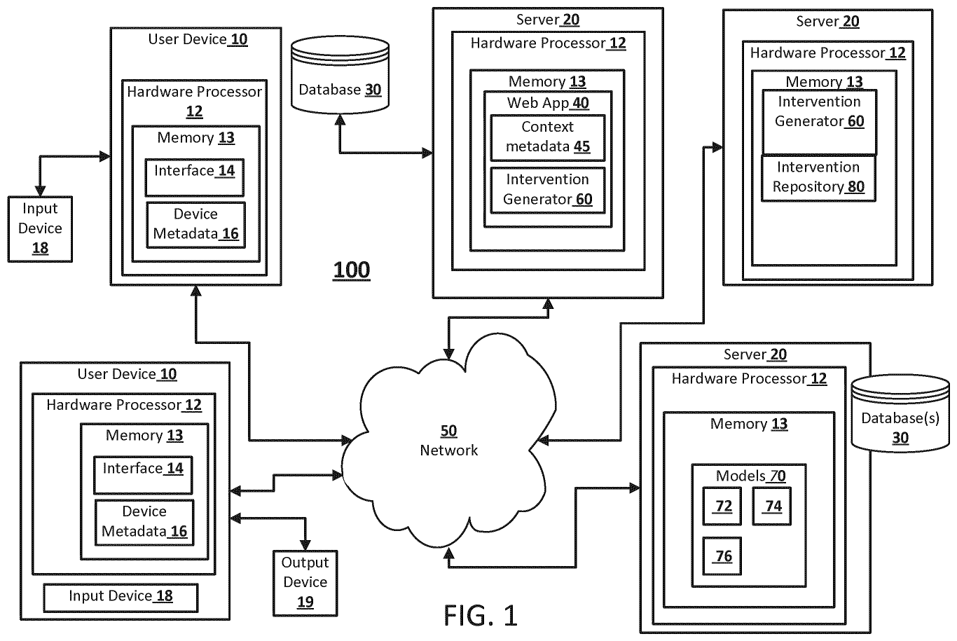

Turning to FIG. 1, there is shown an embodiment of an intervention system 100 that may generate and/or provide one or more individualized interventions based on a wellness model. Intervention system 100 may implement operations of the methods described herein. Intervention system 100 has hardware servers 20, databases 30 stored on non-transitory memory, a network 50, and user devices 10. Servers 20 have hardware processors 12 that are communicatively coupled to databases 30 stored on the non-transitory memory and are operable to access data stored on databases 30. Servers 20 are further communicatively coupled to user devices 10 via network 50 (such as the Internet). Thus, data may be transferred between servers 20 and user devices 10 by transmitting the data using network 50. The user devices 10 include non-transitory computer readable storage medium storing instructions to configure one or more hardware processors 12 to provide an interface 14 for collecting data, and exchanging data and commands with other components of the system 100. The user devices 10 have one or more network interfaces to communicate with network 50 and exchange data with other components of the system 100. The servers 20 may also have a network interface to communicate with network 50 and exchange data with other components of the system 100. The intervention system 100 can apply different computer models (e.g. wellness pattern model, an individualization model, and an intervention model) to generate individualized wellness pattern aware interventions. The computer model can be a machine learning model, predictive model, classification, model, and so on. Example computer models specific to embodiments described herein include a wellness model, an individualization model, and an intervention model. The computer models encode instructions stored in memory and are machine executable to configure one or more hardware processors. That is, the computer model includes encoded machine readable instructions to configure a processor to implement operations to generate output data. The computer model of encoded instructions can be executed by one or more hardware processors to receive input data, process the data, and generate output data indicating an individualized wellness pattern aware intervention. The model can be trained using training data, and updated using feedback data. The machine learning model can be the output of the training process and can be instructions to configure hardware processors to detect patterns and generate predictions as output data. For example, the intervention system 100 may have one or more predictive models to generate output data for indicating an individualized wellness pattern aware intervention as predictions about a user based on input data. The intervention system 100 can apply different computer models to control use of one or more hardware devices to provide the individualized wellness pattern aware intervention. The intervention system 100 can use different computer models for processing different types of input data identifying qualities associated with the intervention, such as digital audio, image or video, speech signals, for example. The intervention system 100 can involve instructions to trigger measurements by different hardware devices to receive or capture the different types of input data. The intervention system 100 can use the computer models to derive or predict the physical state of a user from measurements of physical properties by sensors. The intervention system 100 can generate output data indicating an individualized wellness pattern aware intervention and trigger computer-implemented simulations representing an interaction with an external physical reality using measurements as input data to predict the physical state of the user. In some embodiments, the intervention system 100 (e.g. using one or more hardware processors 12) generates individualization data objects by processing input data identifying qualities associated with the intervention from a simulation environment with one or more digital twins corresponding to one or more physical objects relating to the intervention. The input data can include data relating to at least one of mood and emotion within the simulation environment. The intervention system 100 can use sensors to collect measurements that include data relating to physical physiological modeling.

The intervention system 100 generates individualized interventions using different types of computing models. The intervention system 100 generates individualized interventions based on a wellness model. The wellness model defines one or more multi-state longitudinal journeys for one or more goals. There may be a time duration for an intervention or an amount of time for the multi-state longitudinal journey. In some embodiments, an intervention may not have a defined time duration, and the time of the intervention can vary or change based on shifting wellness objectives of the wellness model. For example, the user's skill or capacity level (or lack thereof) in an activity of an intervention might shift the trajectory or timing. The wellness model can define time related data for the multi-state longitudinal journey, and/or receive time related data as input. The intervention system 100 can implement a training process to generate a trained wellness model. The intervention system 100 updates the wellness model using data from simulation, reinforcement learning, and also output from the wellness model (e.g. feedback loop into itself). For example, the intervention system 100 can use Monte Carlo simulations to estimate possible outcomes of an uncertain event. The intervention system 100 can predict a set of outcomes based on an estimated range of values to build a wellness model of possible outcomes for one or more goals by leveraging probability distributions for variables that have inherent uncertainty, for example. The intervention system 100 can use output from an intervention model as an input for the wellness model. The intervention system 100 can use output from the wellness model as feedback data into the invention model, while also using individualization data for tuning the wellness model over time. The wellness model can define one or more paths for a wellness journey or trajectory to meet a goal set. In some embodiments, wellness model can define a set of paths or patterns for a long multiday wellness journey or trajectory. The intervention system 100 can compute representational values associated with a multi-state longitudinal journey and can calculate one or more wellness pattern trajectories. In embodiments, a wellness model can define one or more wellness patterns or paths. A multi-state longitudinal journey can have a wellness pattern trajectory defined by one or more wellness paths of the model.

The intervention system 100 generates individualized interventions based on an individualization model. The intervention system 100 can generate, store, update, and use multiple individualization models. The intervention system 100 can generate an individualization model corresponding to a user, an individualization model corresponding to multiple users, multiple individualization models corresponding to a user, or multiple individualization models corresponding to multiple users. The individualization model can receive as input data physical physiological user attributes and/or non-physical physiological user attributes. The individualization model can rely on a digital twin representation of the user (or other physical object). The digital twin can represent the user in a simulation environment. The digital twin maps physiological outputs related to the user for the simulation environment. The simulation environment can generate simulation data relating to the digital twin to estimate or predict attributes of the user in a physical environment with similarities to the simulation environment. The simulation environment can generate data relating to mood and/or emotion that can be used as input data into the model(s). The simulation environment can develop or simulate non-physical physiological aspects of the user for the digital twin. For example, the simulation environment can represent non-physical physiological aspects in the digital twin, such as mood, emotion, feel-state, and/or motivation levels. The physiological aspects can be self-reported in the user data, simulated through digital twin self-reporting, extrapolated through physiological measures, extrapolated through analysed behaviours (e.g. starting, completing, not starting, quitting an intervention), and so on. The intervention system 100 can generate one or more individualization data objects by processing input data identifying qualities associated with the intervention from a simulation environment with one or more digital twins corresponding to one or more physical objects relating to the intervention. In some embodiments, the input data from the simulation environment can be data relating to non-physical physiological user attributes, such as data representing mood and/or emotion within the simulation environment. In some embodiments, the input data from the simulation environment can be data relating to physical physiological modeling. The simulation environment can represent physical physiological user attributes and/or non-physical physiological user attributes in the digital twin.

The individualization model can use data from the digital twin to define key points of personalization relevant to a given user. The intervention system 100 can train or update the individualization model using inverse reinforcement learning (e.g. learning goals, values, or rewards of a user by observing its behavior). Further, the user or individual provides feedback data that can be used by intervention system 100 to further train or update the individualization model. The feedback data can represent a user's reflection on the journey. The individualization model does not define a specific goal set, which is different from the wellness model which does relate to a goal set. The intervention system 100 can use output from the individualization model as a feedback loop for the digital twin, which changes the simulation for the digital twin. The intervention system 100 can cohort contextually relevant information of a group of people within their individualization models. Each person or user has a corresponding digital twin for a simulation environment. A group of users can have the same (or similar) data elements (e.g. runner, six foot, new born baby) to populate variables of the model. The system 100 can use the output from the individualization models as an input to simulate the wellness model. The individualization model can involve one or more personality components that can be based on personality factors that measure or estimate different aspects of a user's personality.

The intervention system 100 generates individualized interventions based on an intervention model. In some example embodiments, the intervention system 100 uses a digital twin corresponding to the user to simulate or run through possible interventions in a labeled set. The intervention system 100 can use other intervention data sets in other embodiments to simulate or run through possible interventions. The intervention system 100 uses feedback from possible outcomes of those interventions to determine a next best choice for an intervention. The intervention system 100 improves over each simulation loop to determine the next best action for wellness and/or individualization factors.

An example intervention is an activity. The intervention model can have multiple activity measures. For example, the intervention model can activity measures for different aspects of an activity, such as a user preference for an activity, evaluation of activity effect using a cohort model, evaluation of activity effect using a digital twin model, activity start time, activity frequency, activity trigger or prior action, activity association with a habit, activity duration, activity measure, activity success measure, activity partial success measure, one or more methods of recognizing an activity completion, activity streak calculation, activity hedonic adaptation calculation, activity eudaimonic adaptation, user preference to discontinue a habit, user preference to discontinue an activity, identification of the association between an activity and an unwanted habit, identification of the association between an activity and an unwanted activity, readiness to expand activity calculation, readiness to add a new activity calculation, mapping of related habits, user reflection associated with an activity, user preference associated with the disruption of an activity. The activity measure can include a planned, previous, predicted, or proposed activity value.

The intervention system 100 uses multiple computer models to work together in combination (rather than each model in isolation) to create the intervention experience (e.g. a set of interventions for the journey). The intervention system 100 can use the models to predict not just the next best intervention, but the next best intervention in the context of the longer habit or journey of an individual user (e.g. next best intervention for the individual in the context of a wellness journey). The intervention system 100 uses individualization, reflection, user input (e.g. data from journaling) as an input data or training data for the computer models in addition to the observed behaviour of user or physiological data collected about the user. The intervention system 100 can train, update or fine tune the computer models using feedback data indicating what the user actually feels after the intervention to supplement data captured or detected by sensors and/or physiological results.

The intervention system 100 generates individualized interventions for multiple users. A number of users of intervention system 100 may use user devices 10 to exchange data and commands with servers 20 in manners described in further detail below. For simplicity of illustration, only two user device 10 are shown in FIG. 1, however, system 100 can include multiple user devices 10, or even a single user device 10. The user devices 10 may be the same or different types of devices. The intervention system 100 is not limited to a particular configuration and different combinations of components can be used for different embodiments. Furthermore, while interaction system 100 shows three servers 20 and two databases 30 as an illustrative example, interaction system 100 extends to different numbers of servers 20 and databases 30 (such as a single server communicatively coupled to a single database). The servers 20 can be the same or different types of devices.

The user device 10 has at least one hardware processor 12, a data storage device 13 (including volatile memory or non-volatile memory or other data storage elements or a combination thereof), and at least one communication or network interface 14. The user device 10 components may be connected in various ways including directly coupled or indirectly coupled via a network 50. The user device 10 is configured to carry out the operations of methods described herein.

Each hardware processor 12 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof. Memory 13 may include a suitable combination of any type of computer memory that is located either internally or externally such as.

Each network interface 14 enables computing device 10 to communicate with other components, to exchange data with other components, to access and connect to network resources, to serve applications, and perform other computing applications by connecting to a network 50 (or multiple networks) capable of carrying data. The communication or network interface 14 can enable user device 10 to interconnect with one or more input devices, such as a keyboard, mouse, camera, touch screen and a microphone, or with one or more output devices such as a display screen and a speaker.

The memory 13 can store device metadata 16 which can include available metadata for factors such as memory, processor speed, touch screen, resolution, camera, video camera, processor, device location, haptic input/output devices, augmented reality glasses, virtual reality headsets. The system 100 can determine device capacity for interaction or intervention types by evaluating the device metadata 16, for example.

According to some embodiments, user device 10 is a mobile device such as a smartphone, although in other embodiments user device 10 may be any other suitable device that may be operated and interfaced with by a user. For example, user device 10 may comprise a laptop, a personal computer, an interactive kiosk device, immersive hardware device, smart mirror or a tablet device. User device 10, may include multiple types of user devices and may include a combination of devices such as smart phones, computers, tablet devices, within system 100.

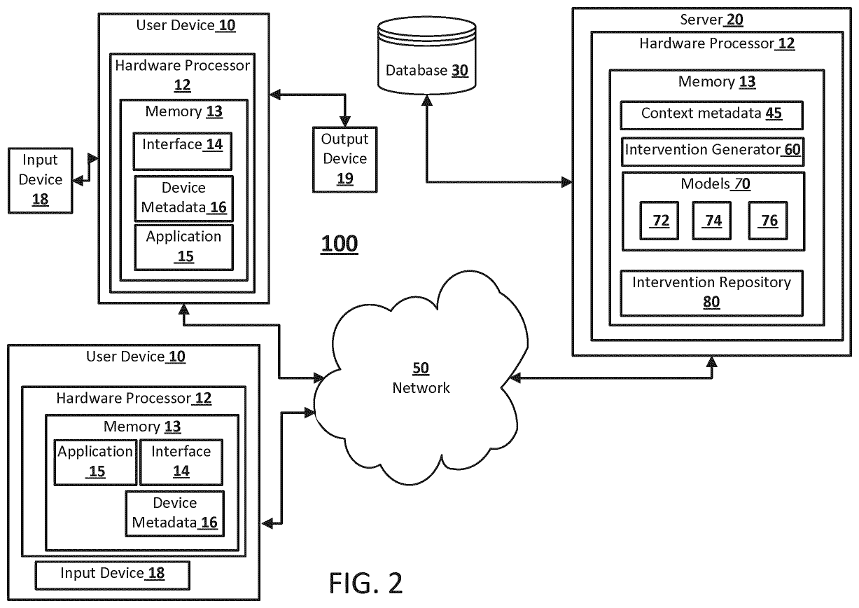

In FIG. 1 the example server architecture includes a server 20 with Web App 40 providing a wellness web application. In other example architectures, similar functionality is provided by application 15 (FIG. 2). Executable instructions or code components such as Intervention Generator 60, Intervention Model 76, and Intervention Repository 80 may be installed on more than one server 20 within system 100. In some example architectures, Intervention Generator 60 may be installed on user device 10.

The server 20 has at least one hardware processor 12, a data storage device 13 (including volatile memory or non-volatile memory or other data storage elements or a combination thereof), and at least one communication or network interface. The server 20 components may be connected in various ways including directly coupled or indirectly coupled via a network 50. The server 20 is configured to carry out the operations of methods described herein.

User device 10 includes input and output capacity (via network interface 14 or I/O interface), a hardware processor 12, and computer-readable medium or memory 13 such as non-transitory computer memory storing computer program code. Input device 18 may be integrated within user device 10 or connected in various ways including directly coupled or indirectly coupled via a network 50. The input device can perform measurements. For example, the input device can include (or couple to) one or more sensors that can capture measurements relating to a user or its environment or context. A hardware processor 12 can receive input data from the measurements. Similarly, output device 19 may be integrated within user device 10 or connected in various ways including directly coupled or indirectly coupled via a network 50. The output device 19 can activate, trigger, or present one or more interventions over a time duration. For example, output device 19 can activate or trigger audio based intervention at a speaker device. As another example, output device 19 can present a visual intervention at a display device. As a further example, output device 19 can trigger lighting devices to provide a visual intervention based on ambient lighting. The hardware processor 12 and/or output device 19 can activate, trigger, start or initiate the intervention by executing instructions that can actuate other components or devices. For example, the hardware processor 12 and/or output device 19 can trigger the execution of code that controls a heating unit on an exercise mat and that also controls a video playback on a display device.

The intervention can involve different types of devices to generate different types of discernible effects to provide a multi-sensory experience. In some embodiments, multiple interventions can be provided over a time period. For example, a first intervention can be provided at a first time, a second intervention can be provided at a second time, and so on. In some embodiments, multiple interventions can be provided simultaneously at a first time, and another intervention can be provided a second time, and so on. User device 10 may be coupled with more than one input device 18, more than one output device 19, and more than one of both input device 18 and output device 19. A single device may contain input device 18 and output device 19 functionality, an example of this would be a connected headset with integrated microphone.

In some embodiments, the function of databases 30 may be implemented by servers 20 with non-transitory storage devices or memory. In other words, servers 20 may store the user data located on databases 30 within internal memory and may additionally perform any of the processing of data described herein. However, in the embodiment of FIG. 1, servers 20 are configured to remotely access the contents of databases 30, or store data on databases 30, when required.

Turning to FIG. 2, there is shown another embodiment of a user device 10 where the application 15 includes executable instructions displaying information concerning providing interventions.

In some embodiments, computer models 70 includes wellness patterns 72, individualization 74, intervention 76 models. These models may be stored in memory 13, database 30. In some embodiments, intervention 76 is integrated in repository 80. The computer models 70 (e.g. machine learning models, predictive models) can receive input data from a simulation environment with a digital twin corresponding to a user, and other digital twins corresponding to other physical objects related to the intervention. The simulation environment can provide data to the various predictive models 70 for use in generating output data for the intervention. The input data can be simulation data or simulated data. The computer-implemented simulation environment can have features representing an interaction with an external physical reality and the input or output may provide a technical effect related to this interaction. The computer-implemented simulation environment can receive measurements (e.g. from sensors) as input to calculate or predict the physical state of an existing real object (e.g. a user, or other object related to the intervention). The output results can be used to generate the intervention that is provided or activated at a user device.

The interaction system 100 evaluates activity (captured as measurements and received as input data) to generate and/or provide an individualized wellness pattern aware intervention, and in conjunction with the intervention generator 60, and in some embodiments models 70, and/or intervention repository 80 evaluates the type of interaction and generates the individualized wellness pattern aware interventions.

In some embodiments, the multisensory digital interaction experience (or intervention) is generated as executable instructions stored within web app 40 or application 18. In some embodiments the individualized wellness pattern aware intervention is streamed to user device 10 through network 50. The user device 10 and/or output device 19 may be a device such as a smart home peripheral device, smart exercise mirror, or an ambient connected device related to lighting, auditory, or olfactory experience.

The individualized wellness pattern aware intervention system 100 has non-transitory memory storing data records, context data, intervention data, individualization data, wellness pattern data, and additional metadata received from a plurality of channels, at servers 20 and databases 30. For example, the data records can involve a wide range of data related to users, user types, user activity, user schedules, wellness patterns, context, activity types, feel-states, and device metadata. The data involves structured data, unstructured data, metadata, text, numeric values, images, biometric data, physiological data, activity data, renderings based on images, video, audio, sensor data, and so on.

For example, the contextual data includes data that pertains to the context for an intervention. For example, in embodiments, contextual data contains data identifying qualities such as specific contextual user data, user classification metadata, user current activity, user historical activity, user physiological data, user previous physiological data, user biometric data, user previous biometric data, user goals, user personality, physical location, availability of exercise equipment, user time zone, user nutrition, user nutrition history, user hydration, user hydration history, ambient temperature, ambient temperature history, current lighting, lighting history, specific contextual retail activity, categories of retail activity, specific contextual activity/movement profile data, categories of activity/movement profile data, specific contextual, specific feel state data, categories of feel state data, specific wellness data, categories of wellness data, specific contextual mood data, categories of mood data, and so on. In some embodiments, input device 18 provides one or more element of the context data.

There will now be described methods for generating and providing individualized interventions for a user device 10 based on wellness patterns, intervention models, individualization, user context, device capacity and preferences, and providing the intervention to the user. The methods can involve performing measurements (e.g. using sensors) relating to a user and/or an environment, and receiving input data from the measurements. The methods can involve triggering, activating, or presenting one or more interventions over a time duration to provide discernible effects, including actuation of physical hardware components. Accordingly, the methods involve computer hardware and physical equipment to perform measurements for the input data, and/or provide discernible effects for the interventions.

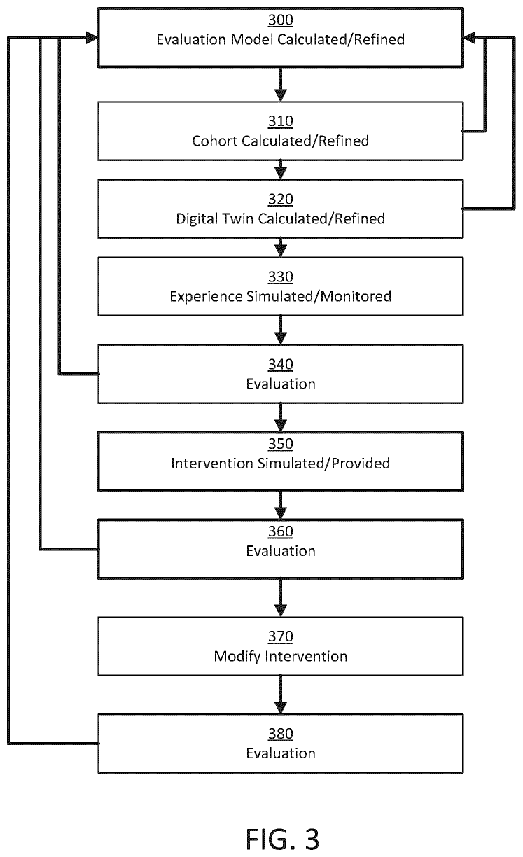

Methods, and aspects of methods are shown generally in FIGS. 3-11 which show diagrams of the steps that may be taken to provide and generate an individualized wellness pattern aware intervention. As the skilled person would recognize, the steps shown in FIGS. 3-11 are exemplary in nature, and the order of the steps may be changed, and steps may be omitted and/or added without departing from the scope of the disclosure. Methods can perform different combinations of operations described herein to provide or generate interventions.

Turning to FIG. 3, in accordance with an embodiment, a method of generating and refining a model for the simulating, generating and providing of individualized interventions is provided. This method is applicable to simulated, actual, and a combination of simulated and actual individualized interventions. In one example embodiment, providing the intervention comprises providing the intervention within a simulation environment which may be used to generate initial hypothetical computer models and to refine models which are based on data from actual user interventions, simulated interventions, or a combination of user interventions and simulated interventions. The method of FIG. 3 applies both to a specific user, a community of users, a simulated representation of an individual user, a digital twin, an accountability partner, a cohort of users, and the like. A digital twin can be a virtual model that accurately reflects a physical object. A simulation environment can involve digital twins of users and different physical object. Sensors can obtain measurements about different aspects of the physical object(s). The intervention system 100 can process the measurements to predict the state of the physical object(s) and generate or update the corresponding digital twin to reflect the predicted state. The digital twin or virtual model can be user to run simulations in the simulation environment to derive output data, that can then be used to individualized wellness pattern aware interventions for the user to impact the external physical reality of a user. A digital twin can be used for multiple simulation environments to generate different types of output or interventions. The intervention system 100 can continuously monitor and obtain measurements about the physical object(s) to continuously update the digital twin and generate new individualized wellness pattern aware interventions, which in turn may impact the state of the physical object and trigger further measurements. Accordingly, intervention system 100 can receive different types of input data, such as simulated data and measurement data.

The intervention system 100 can provide individualized wellness pattern aware interventions using a simulation environment and digital twin representing a user. The intervention system 100 can use measurements as input data to predict the physical state of the user using different computer models (e.g. wellness pattern model, an individualization model, and an intervention model). Individualization data objects can be generated by processing input data identifying qualities associated with the intervention. The computer models of encoded instructions stored in memory and machine executable configure one or more hardware processors to control the simulation environment based on the output data indicating an individualized wellness pattern aware intervention. The intervention system 100 can modify the simulation environment by triggering computer-implemented simulations representing an interaction with an external physical reality using the measurements that predict the physical state of the user. The computer model of encoded instructions can be executed by one or more hardware processors to receive measurements as input data and generate output data indicating an individualized wellness pattern aware intervention. For example, the intervention system 100 may have one or more predictive models to generate output data for indicating an individualized wellness pattern aware intervention as predictions about a user based on the measurements. The intervention system 100 can apply different computer models to control the simulation environment to provide the individualized wellness pattern aware intervention. The intervention system 100 can involve instructions to trigger measurements by different hardware devices to receive or capture the different types of input data. The intervention system 100 can use the computer models to derive or predict the physical state of a user from measurements of physical properties by sensors. The intervention system 100 can use different computer models for processing measurements identifying qualities associated with the intervention.

The method in FIG. 3 may make use of different types of machine learning models based on one or more of a combination of unsupervised, supervised, and reinforcement learning. Such machine learning may be performed using processes and evaluation tools such as K-Means Clustering, Hierarchical Clustering, Anomaly Detection, Principal Component Analysis, APriori Algorithm, Naïve Bayes Classifier, Decision Tree, Logistic Regression, Linear Regression, Regression Tree, K-Nearest Neighbour, AdaBoost, Markov Decision Processes, Linear Bellman Completeness, Policy Gradient, Asynchronous Advantage Actor-Critic (AC3), Trust Region Policy Optimization (TRPO), Proximal Policy Optimization (PPO), Deep Q Neural Network (DQN), C51, Distributional Reinforcement Learning with Quantile Regressions (QR-DQN), Hindsight Experience Replay (HER) and the like. In one embodiment, the machine learning is based on one or more of cohort intervention feedback, accountability partner intervention feedback, user intervention feedback, simulated cohort intervention feedback, simulated accountability partner intervention feedback, simulated digital twin intervention feedback, simulated user feedback.

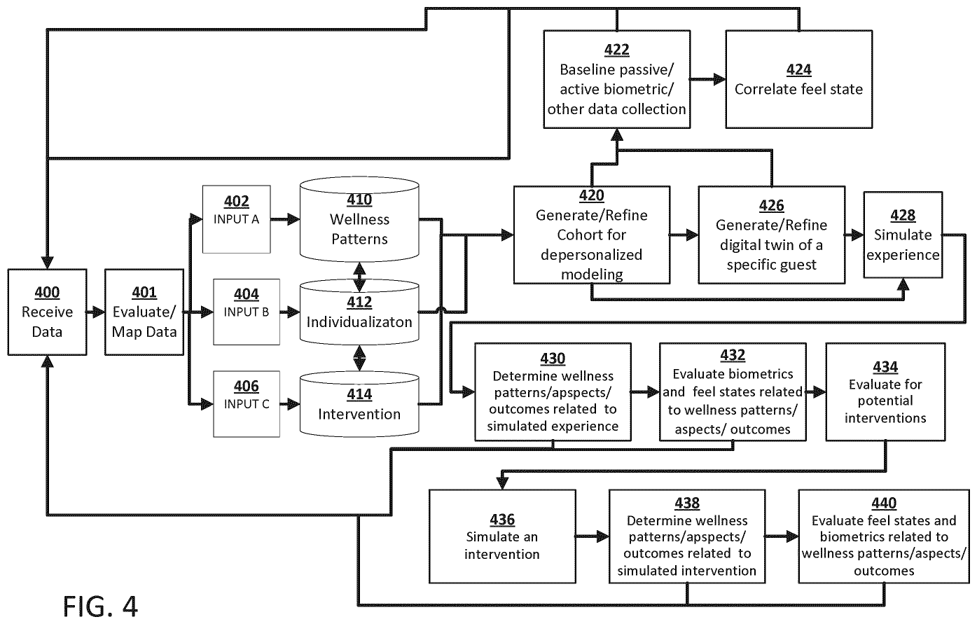

The method in FIG. 3 involves the evaluation model being calculated 300 (e.g. by server 20). In each step in FIG. 3., and generally, calculation or computation may further include recalculation or refinement based on additional data and/or machine learning. FIG. 4 provides additional methods related to the data models that may be involved in the calculation of the evaluation model, in accordance with some embodiments.

A cohort is then calculated 310. One or more cohort is defined to group users and user simulations and model responses to interventions. In some embodiments, cohorts enable depersonalized modeling of activity and intervention patterns. Cohorts may also provide additional longitudinal data that can be mapped to age, life journey, lived experience, and other aspects of a multi-feel state longitudinal journey. In some embodiments, when interventions are generated for a specific human user, one or more cohort data object are used to evaluate the efficacy of a potential intervention given data based on modeling that intervention with one or more cohorts having one or more characteristic in common with the human user. In one embodiment, cohort calculation is performed in conjunction with models 70 including individualization model 74. The calculation of the cohort 310 potentially provides additional data to calculate and refine the evaluation model 300.

A digital twin is calculated 320. A digital twin represents an actual physical user to provide a data object for the purposes of evaluation. The digital twin can be defined by physiological data the represents the user. In addition to digital twins to represent specific human users, in some embodiments, a digital twin is generated to represent specific “individualized” aspects within a cohort data model. A digital twin represents an individualization data object. In one embodiment, digital twin calculation is performed in conjunction with models 70 including individualization model 74 to generate and/or update a digital twin in a simulation environment. The calculation of the digital twin 320 potentially provides additional data to calculate and refine the evaluation model 300. The simulation environment may have multiple digital twins representing other physical objects.

Experience is simulated/monitored 330 in the simulation environment. One or more hardware processors can generate the simulation environment or virtual environment to represent or imitate operation of physical world objects, processes or systems. Simulations of baseline activity are used. An intervention is an action, change in activity, environmental change, feedback, or the like with the intention to change, encourage the maintenance/extension, or end an activity. The baseline activity, which may constitute “inactivity”, or activity unrelated to physical exercise and wellness, provides a context for the intervention and is a potential factor in what intervention types might be applicable. In the case of a human user, activity is monitored with sensors, through extrapolated schedules and activity patterns, participation in fitness activities, GPS data, camera data, microphone data, electrodermal activity such as galvanic skin response (GSR), physiological data and the like. This data may be received from one or more of user input device 18, web app 40, user device 10 application 15.

Evaluation 340 determines what intervention to simulate or provide. This may be performed in conjunction with intervention generator 60, intervention repository 80, models 70, including wellness patterns 72, individualization 74, intervention 76, and the like. Evaluation of the intervention to provide includes evaluating the relevant wellness patterns, the context of the intervention, individualized preferences, current individualized mood, current individualized emotion, individualized mood context, individualized emotion context, historical mood context, historical emotion context, one or more mood associated with an activity, one or more emotion associated with an activity, one or more mood associated with an activity type, one or more emotion associated with an activity type, the historical efficacy of the intervention type, user device 10 capacity to present one or more type of intervention. Evaluation may result in a determination that, based on one or more factor, offering an intervention is statistically improbable to be effective. The evaluation provides further data to calculate and refine the evaluation model 300.

Intervention is simulated or provided 350. For example, the intervention can be transmitted to a user device for activation. In some embodiments a general intervention type is simulated. In some embodiments specific aspects of the intervention, individualization, and/or personalization are simulated.

In an embodiment, the intervention is provided, in whole or part, using a representation of an accountability partner, the behaviour of an accountability partner, suggestion from an accountability partner, shared goals with an accountability partner, encouragement from an accountability partner and the like. The accountability may be a digital simulation, another user, a filtered or modified version of another user, a trainer, an instructor, a digital simulation of a trainer, a digital simulation of an instructor, a filtered or modified version of a trainer, a filtered or modified version of an instructor or the like. A filtered or modified version may make use of asynchronous content including text, sound, voice, video, image, repurposed content including text, sound, voice, video, image or the like.

In an embodiment, the intervention includes one or more of providing one or more point, token, membership or badge which may be exchanged for or used by the user for a reward. Providing the one or more point, token, membership or badge may be combined with additional interventions and/or types of interventions. The reward may be one or more of a physical object, an experience, a fitness class, a duration with a personal trainer or coach, a charitable donation, entry in a raffle, sweepstakes, lottery, or contest, additional functionality within a user application or retail platform, an upgrade to their tier, an upgrade to their service, early access to functionality within a user application or retail platform, early access to purchase one or more product, early access to a discount, access to apparel modification service (such as hemming or tailoring), access to a apparel exchange or reseller program or service, access to additional retail services (such as a personal shopper, preferred return/exchange policy, bespoke retail experiences, trunk or fashion shows), access to a premium version of a service, an employment, contract, or licensing offer, a promotion or cross-promotion of the user's online, social media, business, or other content, an event invitation, a discount on a physical object, set of physical objects, online retail cart or purchase value, in person retail cart or purchase value, an experience, a fitness class, a duration with a personal trainer or coach, additional functionality, an event, or the like.

Evaluation 360 includes monitoring factors such as the effect of the intervention on the activity, physiological measures, the explicit selection/rejection/modification of the intervention, implicit selection/rejection/modification of the intervention, changes to feel-state, triggering of peak experience. This evaluation may include longer duration evaluations such as a subsequent sleep pattern, ongoing engagement in an activity, and the like. Evaluation may include evaluating a provided rating, feedback, reflection, and/or chat content.

Modify intervention 370 includes modifying the intervention within this context or when providing a similar intervention in the future to a different cohort, digital twin, or individual. In one embodiment, the modify intervention 370 includes modification one or more times in rapid succession. In one embodiment, the modification occurs on a more than one instance of a cohort and/or a digital twin which has been generated such that the data objects represent the same context and identifying aspects as the data object to which the initial intervention, the intervention that is now being modified, was initially provided.

Evaluation 380 includes evaluating the efficacy of modifications in 370 when the intervention is simulated or provided 350. Criteria similar to that of step 360 may be applied.

In one embodiment, the system and evaluation methods further comprise using a generative adversarial network (GAN) (e.g. stored in memory 13) for evaluating one or more of intervention perceived ethics, enjoyability, conformance to social expectations, capacity to make a social connection, capacity to provide social recognition, capacity to support a specified goal, originality.

FIG. 4 shows additional aspects of the method of generating and/or providing individualized interventions based on wellness patterns. Input values and data or computer models of FIG. 4 and in embodiment examples are exemplary in nature, and the range of data provided, the order of the steps may be changed. The simulation and digital twin can provide input data to the various predictive models. The models can be machine learning models encoded as computer programs that are executed by hardware processor to recognize patterns in data or make predictions. Machine learning models can be trained using either labeled, unlabeled, or mixed data. The computer model is a set of instructions that the hardware processor executes to generate the output data for the intervention. The system 100 can create a simulation with digital twins to provide input data to the various predictive models. Data elements and steps may be omitted and/or added without departing from the scope of the disclosure. A data model provides coded data (or metadata) about information stored and/or received as input data. A data model can be used by the system 100 to organize elements of data and standardize relationships between elements of data. For example, a data model can indicate that a data element relating to a user can be composed of or linked to a number of other data elements about the user. A data model can also be used to describe physical locations (e.g. in memory) where data elements are stored. An example data model can include classes and attributes or properties, and relationships between the classes and attributes or properties. The data model is a computer model that encodes the relationships between different classes, objects, attributes or properties. A data attribute can include a data type and value. Each entity or class can have one or more properties or attributes. The data model can have instructions or code that indicate relationships or links between the entities and classes, and also between the attributes.

Receive data 400 includes initial population of the model and ongoing data contributions to the model and system. In some embodiments, data received is based on both actual and simulated activities and measures. In some embodiments, separate models/and our systems are used for actual and simulated data values. Receive data 400 can involve performing measurements and then receiving input data using the measurements. For example, sensors can perform measurements relating to the user and/or environment to receive data 400 using the measurements. In some embodiments, some or all of the data is received from one or more of a social network, retail application, membership application, an exercise application, a training application, or the like.

Evaluate/Map data 401 evaluates the applicability of data. In some embodiments, Evaluate/Map data 401 includes data cleaning techniques, parsing unstructured data, evaluating the reliability of the data received, translating or transforming data, and the like.

Input A 402 includes data related to actual baseline passive/active biometric, feel state, key experience, journey measures, simulated baseline passive/active biometric, feel state, key experience, journey measures.

Input B 404 includes data related to the actual user, community, cohort, including depersonalized user, community, and cohort data; simulated user, digital twin, community, cohort data.

Input C 406 includes data related to the effect of actual interventions, of simulated interventions.

Input A 402 is mapped to wellness pattern data models 410, data objects, and/or repositories. Input B 404 is mapped to individualization 412 data models, data objects, and/or repositories. Input C 406 is mapped to intervention 414 data models, data objects, and/or repositories. Wellness patterns 410 Individualization 412 and Intervention 414 are inter-related. In some embodiments there are interdependencies between the models (410, 412, 414).

In one embodiment, the wellness patterns 410 model includes a wellness measure which calculates a value for one or more of positive emotion, engagement, positive relationships, meaning, achievements, health, competence, self-acceptance, social status, social belonging, mood, relatedness, resilience, giving, vitality, optimism, life satisfaction, reduction of negative emotions, increase of positive emotions, autonomy, purpose in life, environmental mastery, personal growth, purpose in life, pleasure, positive affect, emotional stability, social contribution, social integration, social acceptance, and social coherence. In one embodiment, wellness patterns 410 includes data and metadata about habits, habit formation, habit schedule, habit goals, disruption of habits, the association between habits and wellness, and the like.

In one embodiment, the individualization 412 model includes a personality component which measures factors such as of openness, consciousness, extraversion, agreeableness, neuroticism. In some embodiments, additional measures include one or more of dominance. Influence, steadiness, conscientiousness, inducement, introversion, sensing, intuition, thinking, feeling, judging, perceiving, driver, expressive, amiable, and analytical. In one embodiment, the individualization 412 model measures motivation associated with an individual based on a competitiveness scale and a player orientation to self or others scale, and/or a combination of competitiveness and self-other orientation either associated with an individual or an individual in combination with an activity and/or activity type.

In an embodiment, the system's individualization input to the individualization 412 model includes data pertaining to one or more of user specified preference, user specified goal, user specific wellness objective, user specified training plan, user skill baseline, user wellness baseline, user personality baseline, user activity baseline, identifying a potential user cohort, user habit, user activities, contextual data about user habit, contextual data about user activity, user fitness class history, user purchase history, user community participation, user exercise logs, user biometric records.

In an embodiment, the individualization 412 model includes information related to a user context including one or more of previous activities associated with a user comprising one or more of sleep, exercise, hydration, nutrition, meditation, consumption of medication, calories consumed, calories used, supplements, or the like, exposure to sunlight, exposure to cold, exposure to heat. In one embodiment, information about exercise includes one or more measures such as duration, challenge level, exertion, physical context, engagement of specific physical portions of the body, intellectual engagement, physical recovery, cognitive load recovery, stimulation level, social level, instruction type, independent activity, recorded activity, live streamed activity, and the like.

In an embodiment, the individualization 412 model includes information related to a user context, and may include information regarding the cognitive load associated with previous activities. Cognitive load may be calculated based on the intensity, focus, and safety factors associated with previous activities. For example a particularly strenuous workout, learning a new skill or pattern of exercises, navigating risks such as complex terrain or vehicular traffic when road cycling. The cognitive load associated with the history, previous activities associated with a user, digital twin, or cohort and data related to the typical or trends in the user, digital twin, or cohort recovery from cognitive load, physical activity, and other contextual inputs may be used to refine the interventions offered.

In an embodiment, the individualization 412 model includes information related to a user context includes one or more of health kit data, health record data, information regarding pre-existing conditions, physical challenges, accessibility requirements, adaptive behaviours, recovery ratings, stress scores. This user context data may function as an input or measurements to define a digital twin, cohort, or the like and/or an input to the evaluating of potential interventions when one or more of these factors may not be modeled within the digital twin.

In one embodiment, the individualization 412 model includes information related to a user context including lived experience, current location, planned future location, current weather, anticipated weather, scheduled events, sleep schedule, childcare schedule, work schedule, availability of a workout partner, availability of a workout partner at a future time, access to equipment associated with a location, access to equipment associated with a planned future location, access to terrain associated with a location, access to terrain associated with a planned future location.

In one embodiment, the individualization 412 model includes information related physiological affect (for example increased heart rate or the like), subjective interpretation of physiological affect (for example, interpreting increased heart rate as indicative of fear, indicative of excitement, indicative of enhance athletic performance or the like), and/or lived experience (for example previous contexts and/or experiences that have triggered similar physiological affects for example increased heart rate and/or similar subjective interpretation of physiological affects (for example fear).

In one embodiment, the intervention 414 model includes an activity measure comprising a one or more of a user preference for an activity, evaluation of activity effect using a cohort model, evaluation of activity effect using a digital twin model, activity start time, activity frequency, activity trigger or prior action, activity association with a habit, activity duration, activity measure, activity success measure, activity partial success measure, one or more methods of recognizing an activity completion, activity streak calculation, activity hedonic adaptation calculation, activity eudaimonic adaptation, extrinsic motivation adaptation, intrinsic motivation adaptation, resilience adaptation, external reward entitlement adaptation, adaptation to a community, social adaptation, adaptation to elements that previously offered surprise or novelty, user preference to discontinue a habit, user preference to discontinue an activity, identification of the association between an activity and an unwanted habit, identification of the association between an activity and an unwanted activity, readiness to expand activity calculation, readiness to add a new activity calculation, mapping of related habits, user reflection associated with an activity, user preference associated with the disruption of an activity. In one embodiment, the activity measure includes values related to a planned, previous, predicted, or proposed activity value.

In an embodiment, intervention 414 includes metadata that associates an intervention with one or more habit. In one embodiment, intervention 414 includes data and/or metadata associated with one or more of a user preference for an activity, evaluation of activity effect using a cohort model, evaluation of activity effect using a digital twin model, activity start time, activity frequency, activity trigger or prior action, activity association with a habit, activity duration, activity measure, activity success measure, activity partial success measure, one or more methods of recognizing an activity completion, activity streak calculation, activity hedonic adaptation calculation, activity eudaimonic adaptation, extrinsic motivation adaptation, intrinsic motivation adaptation, resilience adaptation, external reward entitlement adaptation, adaptation to a community, social adaptation, adaptation to elements that previously offered surprise or novelty, user preference to discontinue a habit, user preference to discontinue an activity, identification of the association between an activity and an unwanted habit, identification of the association between an activity and an unwanted activity, readiness to expand activity calculation, readiness to add a new activity calculation, mapping of related habits, user reflection associated with an activity, user preference associated with the disruption of an activity.

Based on the models of Wellness patterns 410 Individualization 412 and Intervention 414, a cohort for depersonalized modeling is generated and refined 420. This cohort is used for baseline passive/active biometric other data collection 422, and correlating feel states 424. This data collection and correlating contributes data, metadata, and data structures to the system refinement as data flows to receive data 400. These processes of data collection 422 and correlating feel state 424 generate a baseline for the ongoing evaluation of the cohort and digital twin throughout the process of simulating experiences to generate simulation data for one or more models 70. Baseline evaluations are a factor in valence, hedonic adaptation, and eudaimonic adaptation elements within the individualization modeling.

When the cohort for depersonalized modeling is generated and refined 420, in one embodiment, the cohort is generated to model behavior and intervention for a group of users sharing one or more common type aspects. A cohort may be generated based on an existing community or a community derived based on data and metadata, in order to simulate the effect of an intervention, or to received depersonalized data regarding the effect of an intervention on a community member, or a combination thereof.

When cohort for depersonalized modeling is generated and refined 420, in one embodiment, the one or more common aspect is based on one or more of a shared life experiences (such as a significant life change, positive life experience, trauma, abuse), a shared determinant of health (such as income and social status, education, employment, social support, housing, food insecurity, transportation, environmental factors, health care, discrimination) a shared wellbeing measurement (such as measures of mental health, physical health, and overall quality of life. (self-reported measures of stress, anxiety, depression, and satisfaction with life), a shared sociodemographic factor such as (age, gender, race, ethnicity, education level, and income level), a shared behavioral characteristic and/or measurement (such as physical activity, diet, sleep, substance use, social interaction, coping strategies, stress management, risky behaviors), a shared physiology, and the like.

In addition to the cohort functioning to model data, in one embodiment, it provides an evaluation structure to generate or refine a digital twin 426. This digital twin may be a specific simulated user identity data object based on an evaluation of the characteristics of the cohort, or may be a model for a specific actual user identity, based on the actual user's relationship to cohorts within the system, wherein the cohort augments the data and metadata associated with the specific user to generate the digital twin, or to refine a previously generated digital twin.

Experience is simulated 428 both for the cohort and the digital twin. An intervention takes place in the context of an experience and this simulation may be based on a probable activity given the cohort or digital twin data object characteristics, or may constitute an activity of interest, an activity associated with a feel-state of interest, an activity selected by another technique, an activity selected from a standard set of daily, weekly, monthly activities, and the like.

Determine wellness patterns/aspects/outcomes related to the simulated experience 430 includes evaluating the relationship of the experience to one or more wellness pattern, the evaluation of qualities associated with the experience such as engagement, duration, user impression of the experience, the outcomes include short-, medium-, and long-term effects of the activity. Evaluate biometrics and feel states related to the wellness patterns/aspects/outcomes 432 maps simulated modeled biometric and feel-state data to wellness patterns/aspects/outcomes and evaluates aspects of this data and inter-relationships. As will be appreciated, when the method is applied to a digital twin of a user, actual biometric and feel-state data, where available, may be mapped to wellness patterns/aspects/outcomes. Steps to determine wellness patterns/aspects/outcomes related to the simulated experience 436 and evaluate biometrics and feel states related to the wellness patterns/aspects/outcomes 432 provide inputs to receive data 400.

Evaluate for potential intervention 434 evaluates the current context, which includes the biometrics and feel states related to the wellness patterns/aspects/outcomes maps simulated modeled biometric and feel-state data to wellness patterns/aspects/outcomes modeling and also additional contextual metadata with regard to the digital twin such as preferences, skill, personality, location, time, equipment availability, garments and/or footwear, device capacity, location spatial capacity, activity history, personalization, etc. In one embodiment, evaluating for potential intervention 434 may determine that no intervention should be provided. Evaluate for potential intervention 434 is informed by wellness patterns 410 individualization 412 and intervention 414.

Simulate an intervention 436 may include simulating a type of intervention, simulating an intervention provided on a specific device type or combination of device types, simulating a customization of the intervention, simulating a personalization of the intervention, simulating a modified content of a media file, simulating an intervention in a simulation of a connected environment, simulating an intervention in a specific context such as a game environment, virtual reality environment, augmented reality environment, simulating providing the intervention with and interactive component that allows the selection and/or modification of the intervention, simulating the intervention as a automatic intervention that does not provide an interactive component, and the like. In one embodiment, the intervention contains multiple aspects. A simple example of an intervention with multiple aspects is simultaneously brightening a smart light and adjusting the content of an exercise program.

Determine wellness patterns/aspects/outcomes related to the simulated intervention 438 includes evaluating the relationship of the intervention to one or more wellness pattern, the evaluation of qualities associated with the experience such as engagement, duration, user impression of the experience, the outcomes include short-, medium-, and long-term effects of the activity. Evaluate biometrics and feel states related to the wellness patterns/aspects/outcomes 440 maps simulated modeled biometric and feel-state data to wellness patterns/aspects/outcomes and evaluates aspects of this data and inter-relationships. As will be appreciated, when the method is applied to a digital twin of a user, actual biometric and feel-state data, where available, may be mapped to wellness patterns/aspects/outcomes. Steps to determine wellness patterns/aspects/outcomes related to the simulated intervention 438 and evaluate biometrics and feel states related to the wellness patterns/aspects/outcomes 440 provide inputs to receive data 400.

In an embodiment, the aspects of the method of generating and/or providing individualized interventions based on wellness patterns represented in FIG. 4, are performed within the context of one or more of a digital retail environment, a virtual reality experience with an integrated or associated retail component, an augmented reality experience with an integrated or associated retail component, a membership platform with an integrated or associated retail component, a community platform with an integrated or associated retail component, a hybrid digital and in-person retail environment, or the like. The wellness patterns 410 may include a wellness measure which calculates a value for one or more of positive emotion, engagement, positive relationships, meaning, achievements, health, competence, self-acceptance, social status, social belonging, mood, relatedness, resilience, giving, vitality, optimism, life satisfaction, reduction of negative emotions, increase of positive emotions, autonomy, purpose in life, environmental mastery, personal growth, purpose in life, pleasure, positive affect, emotional stability, social contribution, social integration, social acceptance, and social coherence, or the like associated with acquiring or experiencing a product, service, instruction, workout, or wellness experience. Individualization 412 model may include one or more of navigational patterns, navigational history, purchase patterns, purchase history, eye-tracking data, feedback and ratings provided, profile information, group membership, products associated with a user, wishlist items, in-cart items, or the like. Intervention 414 model may include one or more of suggesting a product, providing a product, providing information about a product, providing personalized information about a product, providing a virtual assistant, customizing a virtual assistant, providing points, providing a navigational path, providing a category of products, filtering a set of products, providing a special offer, or the like.

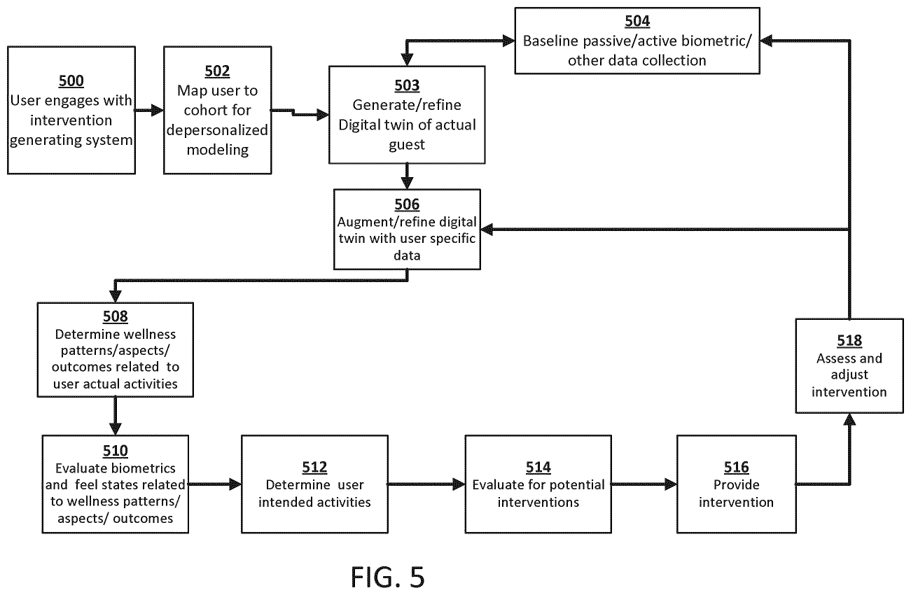

FIG. 5, in accordance with embodiments, shows aspects of the method as the additional aspects of the method of generating and providing individualized intervention based on a wellness pattern when a user engages with the intervention system.

User engages with intervention generating system 500 is triggered when the user implicitly or explicitly requests to engages with the individualized intervention system. In some embodiments, this may constitute a user agreement when participating in a fitness class, studio environment, online retail experience, virtual training environment, community, and the like.

Map user to cohort for depersonalized modeling 502 maps the specific user, based on metadata, questionnaires, biometric data, class history, purchase history, community membership, geographic location, age, other demographic factors, and the like to a cohort of similar individuals for modeling. The model may include data based on simulated cohorts, depersonalized cohorts, and/or a combination.

Generate/refine Digital twin of actual guest 503 generates a specific simulated user identity digital twin data object based on an evaluation. In one embodiment, this digital twin is generated based on the actual user's relationships and similarities to cohorts defined within the system, wherein one or more cohorts augment the data and metadata associated with the digital twin.

Baseline passive/active biometric/other data collection 504 establishes the collection of data related to the user and and/or their digital twin. This includes the collection of biometric and other data pertaining to the user. This data may be collected through different devices such as biometric input devices, audio input devices, video input devices, data and metadata associated with user interaction with other systems, user feedback, user GPS data, and the like. In some embodiments, this data is augmented with simulated data collected based on simulations preformed with the digital twin. Baseline evaluations are a factor in valence, hedonic adaptation, and eudaimonic adaptation extrinsic motivation adaptation, intrinsic motivation adaptation, resilience adaptation, external reward entitlement adaptation, adaptation to a community, social adaptation, adaptation to elements that previously offered surprise or novelty, elements within the individualization modeling.

Augment/refine digital twin with user specific data 506 adds additional data and metadata to the user identity digital twin data object. Additional information is added to the data object which may include refinements based data and metadata related to baseline passive/active biometric other data collection 504, user interactions with interventions, additional customizations provided by the user, additional customizations provided through data and metadata in other systems, and the like.

Determine wellness patterns/aspects/outcomes related to user actual activities 508 includes evaluating the relationship of the user's current activity, activity patterns, and behaviours to one or more wellness pattern, the evaluation of qualities associated with the experience such as engagement, duration, user impression of the experience, the outcomes include short-, medium-, and long-term effects of the activity. In one embodiment, additional factors in evaluating user activity include activity category, skill level, and associated feels states. In one embodiment, receiving data associated with the current activity also includes evaluating the current activity for repetition and frequency which may be associated with user boredom or lack of engagement. In one embodiment, receiving data associated with the current activity also includes evaluating the current activity for an associated mood designation, mood intensity, mood, emotion, emotion intensity, and/or valence measurement.

Evaluate biometrics and feel states related to wellness patterns/aspects/outcomes 510 maps biometric and feel-state data to wellness patterns/aspects/outcomes and evaluates aspects of this data and inter-relationships. As will be appreciated, this method may be augmented with data associated with the digital twin of a user, actual biometric and feel-state data, or a combination.

Determine user intended activities 512 includes determining intended activities using data and metadata related to user historical activity patterns, user current activity, user probable next activity based on current activity, user goals, user habits, user location, user time of day, schedule, context and the like.

Evaluate for potential interventions 514 includes evaluating whether user current activity or user intended activity, including current engagement level, performance level, and the like are currently aligned with an intended wellness pattern. Evaluation also includes comparing current activity to baseline activities. In addition to intended activities, evaluate for potential intervention 514 evaluates the current context, which includes the biometrics and feel states related to the wellness patterns/aspects/outcomes maps simulated modeled biometric and feel-state data to wellness patterns/aspects/outcomes modeling and also additional contextual metadata such as preferences, skill, personality, location, time, equipment availability, garments and/or footwear, device capacity, location spatial capacity, activity history, personalization, etc. In one embodiment, evaluating for potential intervention 514 may determine that no intervention should be provided. Evaluate for potential intervention 434 is informed by wellness patterns 410 individualization 412 and intervention 414.

Provide intervention 516 provides an intervention to the user. This individualized intervention is associated with an intervention type, a specific device or method of providing the intervention which may be a specific device type or combination of device types. In one embodiment, customization and/or personalization may also be applied to the intervention. The intervention may include specific content in a media file, a change in a connected environment, providing the intervention in a specific context such as a game environment, virtual reality environment, augmented reality environment. The intervention may include an interactive component that allows the selection and/or modification of the intervention or providing intervention automatically. In one embodiment, the intervention contains multiple intervention aspects. A simple example of an intervention with multiple aspects is simultaneously brightening a smart light and adjusting the content of an exercise program.

In aspects of embodiments of embodiments, the intervention is generated as executable code instruction modules, interactive media files, interactive applications, and/or applets.

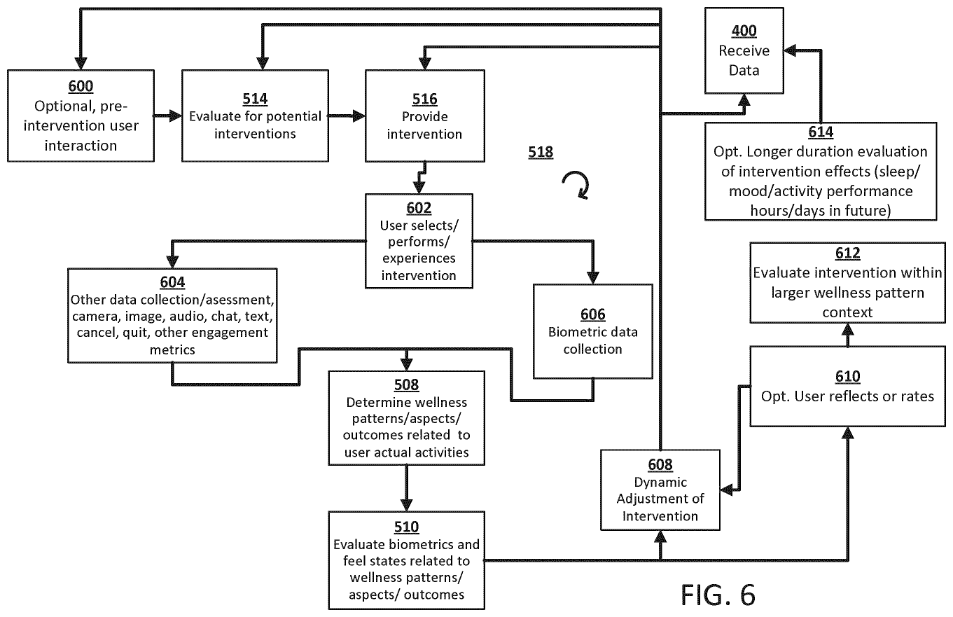

Assess and adjust intervention 518 collects data and metadata about the efficacy of the intervention and optionally offers a modified intervention. The assessment provides data to 504 for further refinement of the digital twin data object. With regard to assessing and adjusting the intervention 518, refer to FIG. 6 for additional processes and methods.

Turning to FIG. 6, in accordance with an embodiment, a method is provided showing additional methods related to providing an individualized intervention, and in particular to FIG. 5 Assess and adjust intervention 518.

Optional, pre-intervention user interaction 600 provides a user interaction engagement step prior to evaluating for the potential intervention 514 and providing the intervention 516. In some embodiments, the user engages in a chat, survey, instructional class interaction to trigger the individualized intervention system processes. This user interaction may be an input to evaluate for potential interventions 514 and provide data and metadata concerning whether an intervention is likely to be effective, the type of intervention the user would prefer, the user capacity for an intervention, and the other data for evaluating the appropriateness of a given intervention to the current context.

Reference FIG. 5 for additional aspects of Evaluate for potential interventions 514 and provide intervention 516.

Steps 602-614 provide further methods related to Assess and adjust intervention 518, FIG. 5.

User selects/performs/experiences intervention 602 includes user interactions with interventions when they are not aware that an intervention is being provided. As indicated provide intervention 516 provides an intervention to the user. In some embodiments, and in some intervention types in some embodiments, the user has an option to accept the individualized intervention. In some embodiments, and in some intervention types in some embodiments, the user has an option to select between intervention options. In some embodiments, and in some intervention types in some embodiments, the intervention is automatically applied. The intervention may include more than one aspect.

This individualized intervention is associated with an intervention type, a specific device or method of providing the intervention which may be a specific device type or combination of device types. In one embodiment, customization and/or personalization may also be applied to the intervention. The intervention may include specific content in a media file, a change in a connected environment, providing the intervention in a specific context such as a game environment, virtual reality environment, augmented reality environment.

Other data collection/assessment, camera, image, audio, chat, text, cancel, quit, other engagement metrics 604 collects data and metadata about the effect of the intervention. This data is evaluated in to determine additional factors, in addition to the intervention, that may affect the data. Data is collected using different methods, such as text entry, physiological metrics, biometric identifiers, video, audio, conversation, image, Global Positioning System (GPS) location, camera input, body mapping with sensors, mapping user position in a physical space, electrodermal activity monitoring, eye tracking, IoT (Internet of Things) input, web browsing history, social media history, purchase history, chat history.

In one embodiment, other data collection/assessment, camera, image, audio, chat, text, cancel, quit, other engagement metrics 604 includes collecting data associated with a mood designation, designation indirectly associated with a mood, emotion, emotion intensity, valence measurement. A user provides an input (through controls, text, voice, images, video, or the like) and this input may be analysed to extrapolate a mood, emotion, intensity of mood, intensity of emotion, or valence rating.

Biometric data collection 606 makes use of different types of available biometric data such as EEG signals, Heart Rate HR, Heart Rate Variability HRV, respiratory rate, blood glucose, oximetry rates, weight, body mass index BMI values, electrodermal activity values, body temperature, pH levels. In some embodiments, cameras, audio sensors, and GPS data are user to extrapolated biometric factors such heart rate, breathing, and shake. Potential biometric sensor sensors include physiological sensor, electrodermal activity sensor such as a galvanic skin response (GSR) sensor, a room sensor, microphone, still camera, video camera, body-based sensor, smart mat based sensor, smart weight based, smart bike based sensor, smart glove based sensor, garment based sensor, smart footwear based sensor, augmented reality headset, virtual reality headset, metaverse headset. Other example sensors include photodetector sensors (for UV and other light exposures), sweat sensors (for sweat volume/duration/context and for detecting compositional elements such as lactate (lactic acid) in particular and/or ammonia, ethanol, ions, glucose, sweat chloride, pH, urea and creatinine, and dehydrations sensors.

Data collection includes passive, active, and a combination of passive and active data collection. In passive data collection, a user does not perform an action that triggers the data collection. In active data collection, the data collection is triggered by user activity. In one embodiment, the user consents to passive data collection from all available input devices.

Determine wellness patterns/aspects/outcomes related to user actual activities 508 includes evaluating the relationship of the user's current activity, activity patterns, and behaviours to one or more wellness pattern, the evaluation of qualities associated with the experience such as engagement, duration, user impression of the experience, the outcomes include short-, medium-, and long-term effects of the activity. In one embodiment, additional factors in evaluating user activity include activity category, skill level, and associated feels states.

Evaluate biometrics and feel states related to wellness patterns/aspects/outcomes 510 maps biometric and feel-state data to wellness patterns/aspects/outcomes and evaluates aspects of this data and inter-relationships. As will be appreciated, this method may be augmented with data associated with the digital twin of a user, actual biometric and feel-state data, or a combination.

Dynamic Adjustment of Intervention 608 updates the intervention based on the interpretation of the date connected in steps 508 and 510. This dynamic adjustment may provide a new intervention type, a change to the existing type, a customization, a personalization, a modification to the wellness pattern applied, and the like. The dynamically adjusted intervention is then provided at 516.

In-some embodiments, an optional, user reflects or rates step 610 precedes the dynamic adjustment 608. This reflecting, rating, and providing qualitative feedback may be based on structured or unstructured data and may be collected using a number of feedback and reflection techniques. In one embodiment, feedback is processed for signals (sentiment, tone, feel state, etc.). In one embodiment, the rating and/or reflection is based on a chat interaction. In one embodiment, the rating and/or reflection is based on the user selecting a graphical representation of their experience.

Evaluate intervention within larger wellness pattern context 612 includes the evaluation of the intervention and the extent to which the intervention was successful in shifting the user's wellness trajectory, feels states, and moments of realization to more closely match a determined wellness pattern.

Optionally, Longer duration evaluation of intervention effects (sleep/mood/activity performance hours/days in future) 614. In some embodiments, the longer duration effects of the intervention are monitored and evaluated. Factors considered may include sleep, mood, activity performance throughout subsequent hours and/or days. In one embodiment, individualized interventions are provided in the context of a multi-day wellness patterns system and the evaluation of interventions over a longer duration is a key factor in determining individualized intervention efficacy.

Individualized intervention data is fed back into the system at step Receive Data 400. See FIG. 4 for methods related to the evaluation and mapping of this data in accordance with some embodiments.

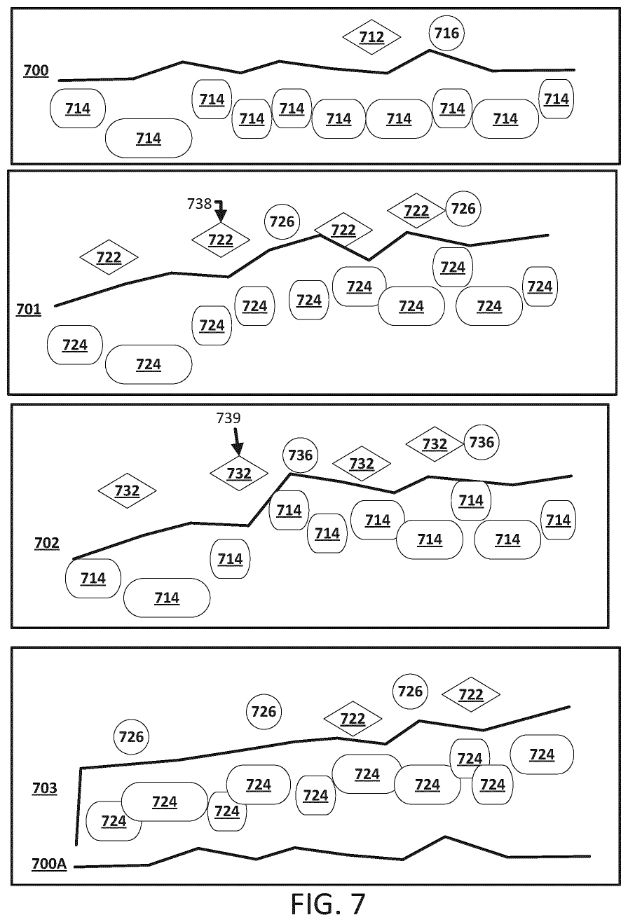

FIGS. 7-9 provide visualizations related to methods in which the multi-feel state longitudinal journey is structured to create a specific type of user experience over a multi-day time duration. Embodiments described, provide an intervention to a user wherein the intervention aligns the user activity and behavior with a larger context, or model, which defines one or more multi-feel state longitudinal journey.

Turning to FIG. 7, a visualization representing high level concepts in the mapping, modeling, generating, and providing of interventions is shown. Line 700 represents a person experiencing a duration with peaks and valleys in the experience of wellness. A number of feel states (714) over the course of the duration 700 are depicted to represent shifting feel-states. The correlation between peak and valleys in wellness over the duration may or may not directly correlate with feel-states. Various positive, neutral, and negative feel-states may arise in response to a number of different factors, including an intervention, but as emotional factors, personality, situational aspects, and the like.

Feels states 714 correspond directly and indirectly to the pattern in 700. Each unit, 714 represents a feel state such as happiness, joy, elation, pleasure, pensiveness, sadness, regret, enthusiasm, engagement, calmness, agitation, frustration, delight, etc. Feel-states are often experienced as being relative to adjacent (previous) feel states. Transitions through feel states often occur in rapid succession for individuals. Given the representational nature of FIG. 7, 702 may serve as simplified representation of a duration that might range from a minute to a lifetime. A duration may also be used to visualize wellness patterns and feel-states associated with a specific activity, such as a yoga practice or running practice in isolation from other user experiences.

A person is able to self-initiate an intervention 712. Common examples of a self initiated intervention include taking a break to change a feel-state when frustrated with an activity, playing an upbeat song to change feel-state, taking a vacation to change an experience of monotony. In some embodiments, self-initiated interventions provide data for generating system-initiated interventions. A person also may have moments of peak realization 716 (physical achievement, insight, skill achievement, spiritual realization, and the like) that arise without an explicit intervention or are related to a self-initiated intervention.

Line 701 represents a digital twin with a duration with peaks and valleys in the experience of wellness. A number of simulated feel states (724) over the course of the duration 700 are depicted to represent shifting feel-states within the simulation. The correlation between peak and valleys in wellness over the duration may or may not directly correlate with positive feel-states which may arise in response to a number of different factors, emotional factors, personality, situational aspects, and the like integrated within the simulated baseline. A number of interventions 722 are modeled in the simulation. Interventions 722 represent one or more intervention type. In one embodiment, intervention 722 is individualized based on a cohort model. In one embodiment, intervention 722 is individualized based on the digital twin's historic data. In one embodiment, simulated intervention 722 is personalized for the digital twin.

These interventions 722, affect the digital twin experience of wellness over duration 701, simulated feel states 724, and the generation of peak realizations 726. Simulated interventions 722, that are evaluated to have a potential for efficacy may then be provided to human user with interventions based on digital twin experience of wellness over duration 702.

As represented in FIG. 7, even if digital twin experience of wellness over duration 701 and user experience of wellness over duration 700 are based on the same data, the introduction of simulated interventions 722 in the digital twin experience of wellness over duration will shift the trajectory of the digital twin experience of wellness over duration 701.

Similarly, when the digital twin simulated experience of wellness over duration 701 is compared to human user with interventions based on digital twin experience of wellness over duration 702 it is evident that interventions may affect the human differently than the digital twin. Humans may make different choices regarding accepting an intervention. That, and a number of other factors contribute to the effect of the intervention 732. As indicated simulated experience 722, labeled 738 for reference, and provided intervention 732 labeled 739 for reference, have different effects on their respective wellness trajectories 701 and 702.

In one embodiment, there is an ideal self model. Trajectory 703 shows the modeling of an ideal self simulation for user 700, also represented as 700A for reference. The ideal self experience of wellness over duration. This trajectory 703 simulates the experience of the user when optimized based on an individualized ideal self model that may include projected changes to activity baseline factors such as sleep, nutrition, meditation, exercise and the like.

Similarly, in one embodiment, a hero model may be generated modeled, where the hero has been selected for or chosen by the user. The hero may be modeled on an actual individual such as an instructor, a celebrity, a coach, an athlete, or an influencer or may be an amalgamation of ideals to provided as an aspirational model. Similarly, in one embodiment, an accountability partner model may be generated or modeled, where the accountability partner has been selected for or chosen by the user. The accountability partner may be modeled on an actual individual such as an instructor, a celebrity, a coach, an athlete, or an influencer, a historic version of an actual individual such as an instructor, a celebrity, a coach, an athlete, or an influencer, or may be an amalgamation of ideals, fitness goals, aptitudes provided as a companionship model.

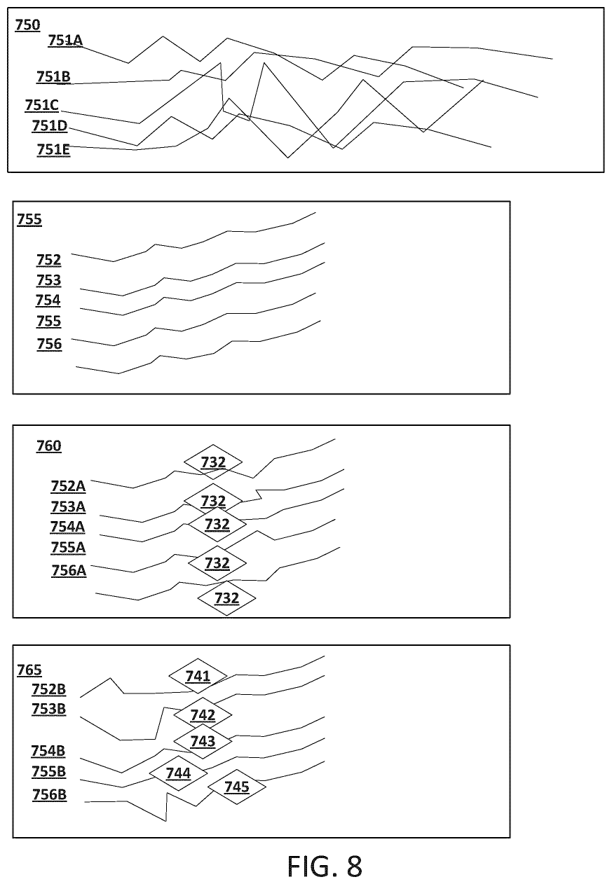

Turning to FIG. 8, a visualization representing high level concepts, in particular related to cohorts and communities, in the mapping, modeling, generating, and providing of interventions is shown. For the purposes of visual simplification, feel-states of type actual 714 and type simulated 724 are omitted from the diagram, but are associated with the trajectories 751A-E, 752A-756A, 752B-756B, 752C-756C.

When evaluating potential interventions, some embodiments include methods for generating a cohort for depersonalized modeling. A user, or digital twin, may be mapped to a cohort based on metadata, questionnaires, biometric data, class history, activity, skill level, purchase history, community membership, geographic location, age, other demographic factors, and the like. The cohort then can be used, either through simulations, or an actual user data cohort associated user data collection, or a combination to evaluate the potential efficacy of a proposed intervention for a specific user, their digital twin, or a community.

A community, functions as a representation of a group of users. These communities may be defined based on metadata, questionnaires, biometric data, class history, current activity, skill level, purchase history, group membership, geographic location, age, activity skill (beginner runner, advanced yogi, etc.), retail search history, activity history, family, friendship group, social media links, academic affiliation, professional affiliation, employment affiliation, shared belief, other demographic factors, and the like. In one embodiment, a community is based on one or more of a shared life experience (such as a significant life change, positive life experience, trauma, abuse), a shared determinant of health (such as income and social status, education, employment, social support, housing, food insecurity, transportation, environmental factors, health care, discrimination) a shared wellbeing measurement (such as measures of mental health, physical health, and overall quality of life. (self-reported measures of stress, anxiety, depression, and satisfaction with life), a shared physiology, a shared sociodemographic factor such as (age, gender, race, ethnicity, education level, and income level), a shared behavioral characteristic and/or measurement (such as physical activity, diet, sleep, substance use, social interaction, coping strategies, stress management, risky behaviors) and the like.

Information about a community, can be an input for an intervention. For example, some interventions provide a user with feedback concerning how they compare with others in the community with an intervention designed to shift a user activity or feel-state. For example, for a user with competitive personality aspects, displaying a metric that shows the user's achievement is greater than others in a community, may trigger an elated feel-state. For another user, with who is highly cooperative seeing that x members of a running community have completed their daily run miles, may motivate the user, not wanting to let down the community, to complete their daily run miles.

A cohort is defined, in example embodiments, to model behavior and intervention for a group of users sharing one or more common type aspects. In one embodiment, a cohort may be generated based on an existing community, similar data associated with a user or digital twin, similar metadata associated with one or more user or digital twin. The cohort may be used to simulate the effect of an intervention, or to received depersonalized data regarding the effect of an intervention on a community member, or a combination thereof.

Communities and cohorts may share similar wellness trajectories where the wellness pattern, feel-state pattern, and placement of realizations are similar. For example, within a meditation class individual participants may, to a greater or lesser degree, through the shared experience, have similar wellness patterns such as those depicted in 755. However, in many communities and cohorts, individual wellness patterns may be very divergent. For example, the community or cohort modeled in 750 shows these divergent patterns. This would be expected in communities and cohorts where individuals are not experiencing shared experiences or individualized wellness pattern aware interventions directed toward the same, or a similar, wellness pattern.

Example 760 provides a visualization of an intervention within a coherent wellness trajectory cohort or community in which the intervention disrupts the coherence. This may be a result of offering a different type of individualized intervention, or in the example provided, intervention 732 for each of 752A-756A generates a distinctive response to the same intervention.

In contrast, example 765, provides a visualization of community or cohort with limit initial coherence in wellness trajectory being moved into a coherent wellness trajectory through individualized interventions 741-745.

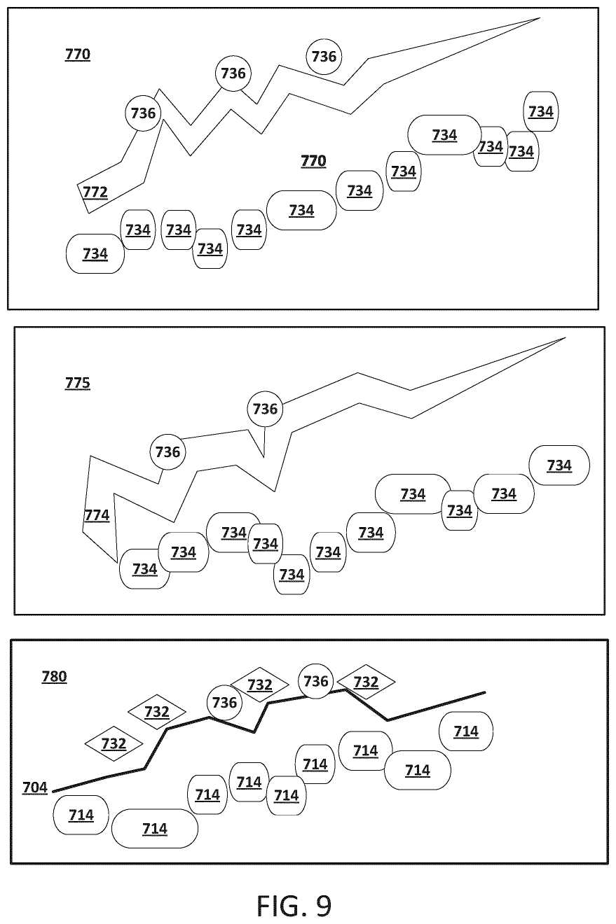

FIG. 9 shows a visualization representing high level concepts in the mapping, modeling, generating, and providing of interventions. Wellness pattern 770 and wellness pattern 775 provide examples models for two different multi-feel state longitudinal journey that extend over multiple days. It should be appreciated, that these example models are provided for the purpose of illustration and there exist a large number of such patterns, each with potential customization to match specific wellness objectives, and customization to reflect individualization and such aspects as valence, personality, skill, baseline activity, and the like.

The multi-feel state longitudinal journey is structured to create a specific type of user experience over a multi-day time duration. By providing interventions that are consistent with the multi-feel state longitudinal journey, and the shifts in user activity required for the multi-feel state longitudinal journey, the user is nudged and/or rewarded to engage in behaviors that are consistent with a longer duration wellness model.

In some embodiments, interventions are provided to nudge, support, and/or reward a user with the context of a multi-feel state longitudinal journey that extends over multiple days. Wellness is understood as representing both prevalence of positively perceived feel-states and a perceived positive trajectory within feel-states over a duration.

In one embodiment, the wellness pattern designed to create a specific type of user experience over a multi-day time duration is associated with an “archetypal” pattern. In one aspect of the embodiment, the archetypal pattern is one of, a quest for identity, a quest to find a ideal location, emotional state, or sense of spiritual realization, a quest for justice, a quest to help a community member, a quest for social connection, a quest for social acceptance within a group, a quest for a role or status designation, a quest in search of knowledge, competence or skill, a quest for acceptance and personal affirmation, a quest for transformation, a quest for self-actualization, a quest for pleasure, fun, surprise and adventure, a quest to remove a danger, a quest for a symbolic or metaphoric goal. In an embodiment, the multi-feel state longitudinal journey designed to create a specific type of user experience over a multi-day time duration is associated with a pattern for adherence to a wellness, fitness lifestyle, basic health criteria, training plan, nutritional plan, and interventions are provided that are consistent with generating and improving the individual's adherence to the overall plan.

In some embodiments, a specific wellness pattern is used to generate control instructions to guide the logic for what individualized intervention to offer, how to offer it, when to offer it, in order to generate user behaviors that more closely align with the determined wellness pattern. A wellness model can be used to define different wellness patterns or paths for a longitudinal journey over a time duration. In one embodiment, the selection of the wellness pattern is based on the user's explicit expressed, goals, preferences, and values. A wellness model can be linked to a set of goals defined by the user or generated based on user data. In an embodiment, the selection of the wellness pattern is inferred based on the individualization model 412 and/or cohort and/or digital twin.

In some embodiments, the wellness pattern includes a shift from a primarily extrinsic set of user motivations to a primarily intrinsic set of user motivations. In some embodiments, the wellness pattern incorporates an accountability partner, cohort, or team associated with the pattern logic.

A selected set of wellness patterns may provide a generalized sense of pattern logic for interventions, but the actual interventions provided to nudge the user's wellness pattern closer to the selected pattern are individualized. Individuals have different personalities, pretences, skills, goals, user devices 10, and the like that are factors in whether an intervention is viable and/or likely be effective in shifting user wellness to more closely match the selected wellness pattern. For example, that the same social experience presented to an introverted individual and an extroverted individual, may result in significantly different feel-states.

Returning to FIG. 9, intervention pattern 780 shows a series of interventions in duration 704 (based on initial FIG. 7, 700 duration) designed to more closely align duration 704 to wellness pattern 775. Through the application of interventions 732 trajectory 704 begins to take on the same flat peak pattern of 774 and elevation pattern in feel-states 734. Realization points 736 are also triggered on the trajectory 704. In the example, as is expected, the pattern in 780 shifts towards the shape of 775 but trajectory 704 does not fully match 774 and the placement of realization moments 736 and feel-state elevations 714 are approximate. Trajectory 704 does take a shape more similar to 775 than to wellness pattern 770 which is not the target wellness pattern for 780 with interventions 732.

In one embodiment, multiple wellness patterns inform the evaluation of interventions to provide to a given user during the same duration. In one embodiment, a series of wellness patterns inform the evaluation of interventions to provide to a given user sequentially over a duration. In one embodiment, a specific wellness pattern is applied to a specific activity type, and/or set of activity types, for a given user. In one embodiment, the wellness pattern is based on skill acquisition, increased performance achievement levels, training for an athletic goal, training for a performance or event.

In one embodiment, the wellness pattern is based on a series of behaviors, or habits. Habits can be understood as including the repetition of a specific activity or behavior or the repetition of an activity in a shared behavior category. For example, a habit might be specific, such as swimming every evening for 20 minutes, or it might be engaging in an activity in a shared behavior category, exercising for 20 minutes or more every evening, where the exercise might be running, swimming, or completing a fitness class. Eating a meal, ratio of protein, or number of calories have a high degree of variability in how the intended habit may be completed. A habit may be connected to a specific time of day, a preceding activity, a physical location, number of times per duration (for example one a day, three times a week, less than twice a month, once each quarter) or portion of duration (for example, seven hours per night, 2 hours a week, etc.). In an embodiment, the intervention provides a replacement behavior for an unwanted behavior, or bad habit. In some embodiments, the user is provided with interventions enable them to visualize their habits within the longer duration multi-feel state longitudinal journey.

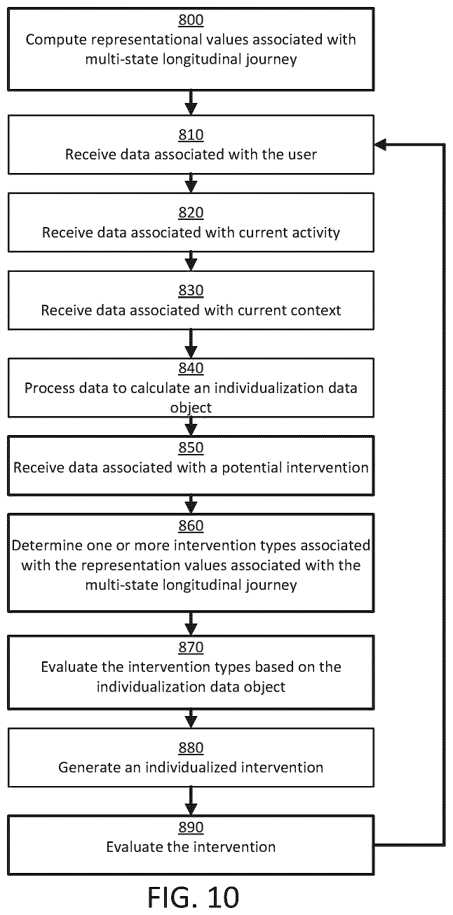

FIG. 10 shows a method for generating individualized wellness pattern aware interventions, in accordance with an embodiment.

Compute representational values associated with multi-state longitudinal journey 800 calculates one or more wellness pattern trajectories. In embodiments, there are wellness patterns associated with a number of wellness models, life narratives and the like. A wellness pattern trajectory may be computed based on data and metadata associated with user activity, or data and metadata based on simulations, or a combination.

Receive data associated with the user 810, includes one or more of receiving actual user data and metadata, receiving data and metadata associated with a digital twin, receiving data and metadata associated with a cohort, receiving data and metadata associated with a community. Determine wellness patterns/aspects/outcomes related to user actual activities.

Receive data associated with the current activity 820 includes evaluating the relationship of the user's current activity, activity patterns, and behaviours to one or more wellness pattern, the evaluation of qualities associated with the experience such as engagement, duration, user impression of the experience, the outcomes include short-, medium-, and long-term effects of the activity. In one embodiment, additional factors in evaluating user activity include activity category, skill level, and associated feels states.

Receive data associated with the current context 830 evaluates the current context, which includes the biometrics and feel states related to the wellness patterns/aspects/outcomes maps simulated modeled biometric and feel-state data to wellness patterns/aspects/outcomes modeling and also additional contextual metadata such as preferences, skill, personality, location, time, equipment availability, garments and/or footwear, device capacity, location spatial capacity, activity history, personalization, etc. In one embodiment, receiving data associated with the current context also includes evaluating the current activity for repetition and frequency which may be associated with user boredom or lack of engagement.

Process data to calculate an individualization data object 840 calculates the individualization data object based on data associated with the user, current activity, and current context. A data model can represent individualization data objects, data flow between the data objects, and interrelationships between data objects to instruct one or more computer devices. A individualization data object can define a location or region of memory or storage device that contains a collection of attributes or groups of values that define aspects of an individual or user. Attributes can define properties, qualities, or characteristics of the individualization data object. An attribute can be a data value, a set of values, or a range of values. The data model can define relationships or connections between different individualization data objects. An individualization data object can define one or more regions of memory or a storage device that contains a data value, data point, or data element or a collection of data values, data points, or data elements that measure aspects of the individual or user. An individualization data object can be associated with identifiers for its data values for use by a computer to access the storage location for the values. An individualization data object can have an identifier. An individualization data object can have a data type. The individualization data object is used to evaluate interventions (e.g. using intervention model) for potential applicability and/or efficacy. In some embodiments, process data to calculate an individualization data object 840 includes calculating a new individualized data object, updating, refining and/or evolving an existing data object with updated values, and/or a combination of calculating new objects and updating objects. As is indicated in the circular depiction in the diagram, in some embodiments, these calculations are continuously updating, maturing and/or evolving existing individualized data objects, while also recognizing and introducing net new individual data objects into the cycle.

In one embodiment, the individualization data object includes subjective, physiological, and preference metadata. In one embodiment, this metadata is evaluated against a wellness model with aspects related to one or more of positive emotion, engagement, relationships, meaningfulness, accomplishment, and health. In one embodiment, a numerical rating is associated with the wellness model aspects.

Receive data associated with a potential intervention 850 provides data about the potential intervention type, intervention dependencies on a location, device, or other equipment, data to enable evaluating the intervention in relationship to user current activity, baseline activity, or user intended activity, including current engagement level, performance level, and the like, and alignment with an intended wellness pattern. Evaluation also includes comparing current activity to baseline activities.

In one embodiment, the intervention data object is evaluated against a wellness model with aspects related to one or more of positive emotion, engagement, relationships, meaningfulness, accomplishment, and health. In one embodiment, a numerical rating is associated with the wellness model aspects. In one embodiment, the individualization data object and intervention data object are evaluated in combination to determine an intervention's capacity for valence. This valence evaluation may be related to shifting one or more of the user's current feel-state, the user's feel state to match a state within an associated multi-state longitudinal journey, a user's physiological state, a user's preferences, a user's relationships, a user's accomplishments, a user's engagement, a user's emotional state, a user's sense of meaning, a user's health.

Determine one or more intervention types associated with the representation values associated with the multi-state longitudinal journey 860 evaluates interventions which are applicable to current location a wellness trajectory in respect to the larger intended wellness pattern.

Evaluate the intervention types based on the individualization data object 870 evaluates the interventions associated with the representation values associated with the multi-state longitudinal journey to determine which interventions are applicable, or potentially effective, given the specific individual, and their current activity, and context.

Generate an individualized intervention 880 includes generating instructions for providing the individualized intervention. The individualized intervention may be provided by a number of devices and/or methods described elsewhere.

Evaluate the intervention 890 evaluates the intervention and its efficacy in relationship to a wellness pattern. In one embodiment, machine learning is used to evaluate the intervention. In one embodiment, evaluating the intervention includes evaluating all, or a subset, of the input and/or data points encountered throughout the current journey leading to the intervention. In one embodiment, evaluating includes evaluating all, or a subset, of the input and/or data points received leading to, during, and post intervention. In one embodiment, evaluating the intervention includes evaluating based past journeys, and/or patterns derived from past journeys. In one embodiment, evaluating includes evaluating based evaluating, all or a subset, of the data objects associated that are new or updated since a previous intervention and/or the completion of a previous journey.

In an embodiment, a user's individualized multi-state longitudinal journey may be generated within the context of a community or cohort multi-state longitudinal journey in which there are shared, parallel, complementary, supportive, and/or competitive activities that define both individualized multi-state longitudinal and/or the cohort multi-state longitudinal journey. For example, in one embodiment users are provided with a shared, or partially shared, journey which uses the collective contributions of more than one user's activity, activity characteristic, activity measures, activity duration, activity completion, habit, habit characteristic, habit measure, habit duration, habit completion, encouragement behaviours, ideas, repetitions, achievements, partial achievements, habit streak, and/or the like. In an embodiment, the user's individualized multi-state longitudinal journey may shift and vary between individual focus and community focus and/or social focus activities, series of activities, activity types, activity categories, goals, interventions, intervention types, and quests.

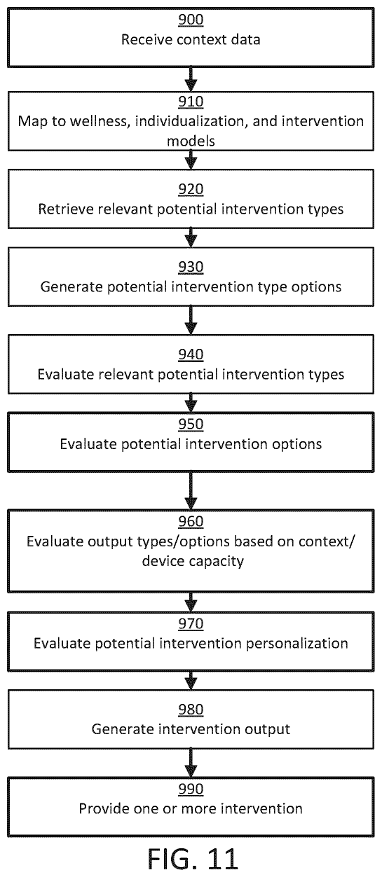

FIG. 11 shows a method for providing individualized wellness pattern aware interventions, in accordance with an embodiment. FIG. 11, Receive content data 900 includes receiving data that identifies the user, current activity, and user device capacity. In some embodiments, additional data and metadata concerning the user, user feel-state, user emotion, user mood, user location, user biometrics, current activity, activity context, equipment, and the like may also be provided.

Map to wellness, individualization, and intervention models 910 represents the step that maps and evaluates the data received 900 and evaluates it against the wellness model, individualization model, and intervention model. In some embodiments, refer to FIG. 4, mapping individualization includes generating and/or refining a cohort and/or a digital twin. In some embodiments, wellness patterns mapping includes generating a baseline and correlating feel states.

Retrieve relevant potential intervention types 920 retrieves relevant intervention types. In one embodiment, potential intervention types are stored in model 414; in one embodiment intervention repository 80; in one embodiment another database 30.

Generate potential intervention type options 930 generates types of interventions that may be offered. Various embodiments provide different intervention categories associated by the function of the intervention in effected user activity, the device or method of delivering the intervention, the feel state associated with the intervention, the probability of the intervention effecting a significant change, and the like.

Evaluate relevant potential intervention types 940 evaluates intervention types in relationship to the wellness, individualization, and intervention models.

Evaluate potential intervention options 950 evaluates the options that exist in how an intervention is provided. For example, a simple encouragement at the start of a workout after skipping a workout intervention could be presented as a badge, fun video, customization of the class, audio or musical feedback, or disco light effect from a connected light feature. Each of these, represents a different way of provided encouragement. These all constitute options for this encouragement intervention.

Evaluate output types/options based on context/960 includes device capacity, location, the availability of space, the availability of equipment, time of day and the like.

Evaluate potential intervention personalization 970 includes one or more of determining whether or not the intervention type has the capacity for personalization, which types of personalization are available, whether or not personalization would improve the efficacy of the intervention, what personalization type would be most effective in the intervention. Personalization can include augmenting the intervention with personalization such as the user's name, the username, age, gender, user's birthday, user's personal goals, user's hero, user's motivational texts, user's badges, user's status, avatar preferences, color preferences, music preferences, location, preferred studio, preferred store, income, family, pets, education level and the like.

Generate intervention output 980 generates instructions for the intervention that are supplied to the appropriate application, device or environment.

Provide one or more intervention 990 provides an intervention to the user. The intervention may include specific content in a media file, a change in a connected environment, providing the intervention in a specific context such as a game environment, virtual reality environment, augmented reality environment. The intervention may include an interactive component that allows the selection and/or modification of the intervention or providing intervention automatically. In one embodiment, the intervention contains multiple intervention aspects.

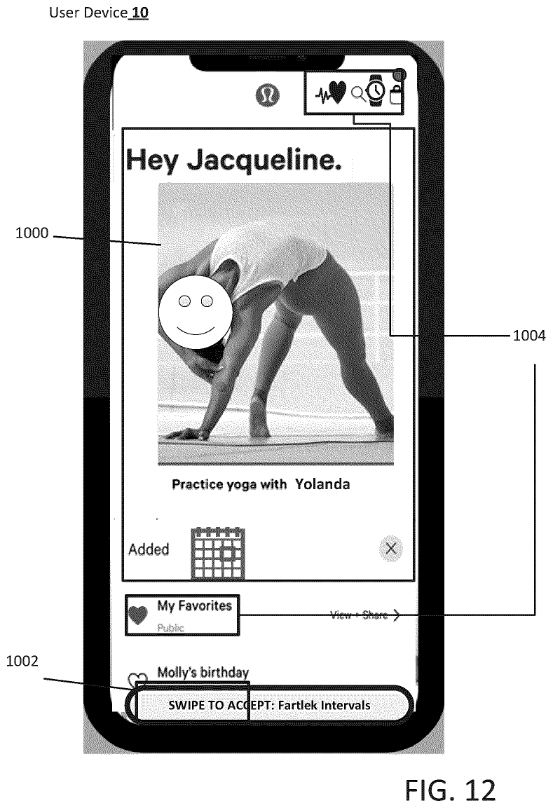

FIGS. 12-15 illustrate examples of aspects of embodiments for generating and providing individualized wellness pattern aware interventions. These are example embodiments, and do not describe the entire scope of all aspects. Other aspects, features and advantages will be apparent to those of ordinary skill in the art upon review of the following description of specific example embodiments.

In some embodiments an interface (e.g., application 15 of user device 10, web app 40 of server 20) provides an intervention. In some embodiments, the intervention is provided through a connected device, or smart device. In some embodiments, the intervention is provided within the context of an exercise environment, exercise class, meditation class, guided workout, guided meditation, or the like by adjusting the content, speed, repetitions, intensity, recommendations contained in the instructional content, the appearance of the instructor, background, and the like, and/or displaying additional feedback, statistics, progress indicators and the like. In some embodiments, the individualized wellness pattern aware intervention, is an independent executable code application.

In one embodiment, activity data includes “offline” data about activity, and behavior that is collected manually, by a device that is not connected to the system, or through images, and the like. In one embodiment, intervention data includes “offline” data that is collected manually, by a device that is not connected to the system, or through images, and the like. In one embodiment, the manually collected data is related to an individual; in one embodiment to a group of individuals; in one embodiment to a combination of individuals and groups. This data may be manually entered through a text upload, an application, a voice prompt chat, scanned as an image, uploaded as a video, and the like. This data is received into the system. See FIG. 4, Receive Data 400. In one embodiment, the activity and/or intervention data is derived from a race, athletic event, sports game, competition, game, meditation, or the like. In one embodiment, the activity and/or intervention data is derived from a class, one on one coaching session, therapy session, or conversation.

In one embodiment, the intervention is provided as human-readable instructions and/or guidance applicable to a real-world 3D context. In one embodiment, the instructions are provided to a coach, leader, teacher, or instructor in order for that coach, leader, teacher, or instructor to communicate the instructions to another individual in-person and/or through a live voice and/or video chat. The intervention may be provided by one or more of a web application, an application installed on a user device, a smart mirror device, a connected music system, a connected lighting system, a connected exercise mat, a connected heating device, a connected cooling device, a connected smell diffuser device, a connected electrical stimulation device, a connected implanted medical device, a virtual reality headset, an augmented reality headset, a metaverse headset, a haptic glove, a game controller, a haptic garment, a retail application, a coaching application, a fitness class or studio application, a meditation application a retail application, a meal or nutritional supplement delivery service application, an email system application, a text message system application, notification system application. Interventions may be provided in a “real-life” 3D reality environment, augmented reality environment, simulated reality environment, virtual reality environment, a game environment, a metaverse environment. An intervention may be provided in a combination of environments. Additionally, the individualized wellness pattern aware intervention may be provided by one or more of a smart watch, a item of smart jewelry (such as a ring, necklace, bracelet, anklet, piercing jewelry, or the like), a item of smart apparel, a smart exercise bike, a smart gym, a smart weight, a smart lighting system, a smart audio system, a tablet, a computer, a device notification, a connected device such as a yoga mat, watch, heart rate monitor, breathing monitor, a blood glucose monitor, an electronic implant, an EEG, a brain-computer interface. A hologram projection system, an autostereoscopic projection system, a smart technology enabled event, a smart technology enabled fitness class, a-smart vehicle, an augmented reality headset, a virtual reality headset, a metaverse headset, a game environment, a haptic glove, a haptic garment, a haptic footwear.

In one embodiment, the individualized intervention system is integrated within a mapped recommendation system. This mapped recommendation system may be related to medical, well-being, or wellness, fitness activity tracking, mood management, product selection, product purchasing, class selection, class purchasing, or any process in which an individual is provided with a recommendation based on a defined logical mapping between data input in a form/survey and a recommendation. In one embodiment, when the individualized intervention system is integrated in a mapped recommendation system, both the individualized intervention and the mapped recommendation are provided to the user. In one embodiment, the mapped recommendation and the individualized intervention are evaluated against criteria to determine whether to provide the mapped recommendation, the individualized intervention, or a combination of the individualized intervention and the mapped recommendation. In one embodiment, summary data concerning the individual data input, the individualized intervention provided, and/or the mapped recommendation provided is generated for a practitioner or organization.

FIG. 12 shows an example embodiment in which individualized wellness pattern aware interventions may be displayed on an application 15 on user device 10. Individualized interventions are distinct from standardized content which is not individualized or wellness pattern aware. Generally streamed/available content, static content based on preferences, standardized personalization and/or notifications based on generic content or a standardized calendar, are not inherently within the scope individualized interventions, although in some cases an individualized intervention may trigger the display of such content.

In this example, there are two interventions displayed to our user Jacqueline. There is an indicator for an exercise class which has been added to her calendar 1000. This indicator has been personalized with her name. Jacqueline does not need to do anything as the intervention, scheduling a class with a preferred instructor next week on a day she often skips her workout, has already been done for her in order to support her longitudinal wellness journey which includes specific fitness goals. The individualized wellness pattern aware intervention is applied automatically.

In this example, a second intervention is also provided. The SWIPE TO ACCEPT intervention 1002, offers Jacqueline the option to add Fartlek intervals to the guidance for her current run. In this example embodiment, this is implemented through a modification to voice guidance and workout music. This intervention is context aware, based on location data and other metadata that Jacqueline is currently having a run and that her pace and engagement with the run is lower than average. Historically, Jacqueline has not engaged well to automatic modifications to her run and has engaged better when provided with an option to accept an individualized wellness pattern aware intervention which she chooses to accept 68% of the time. This intervention is of a type that changes the intensity of a current activity, provides audio feedback, changes music, has a design that makes it easy to accept. In an embodiment, the swipe-able control availability is simultaneously indicated by an audio tone and the user has the option of using a voice command to initiate the intervention option rather than the swipe-able control.

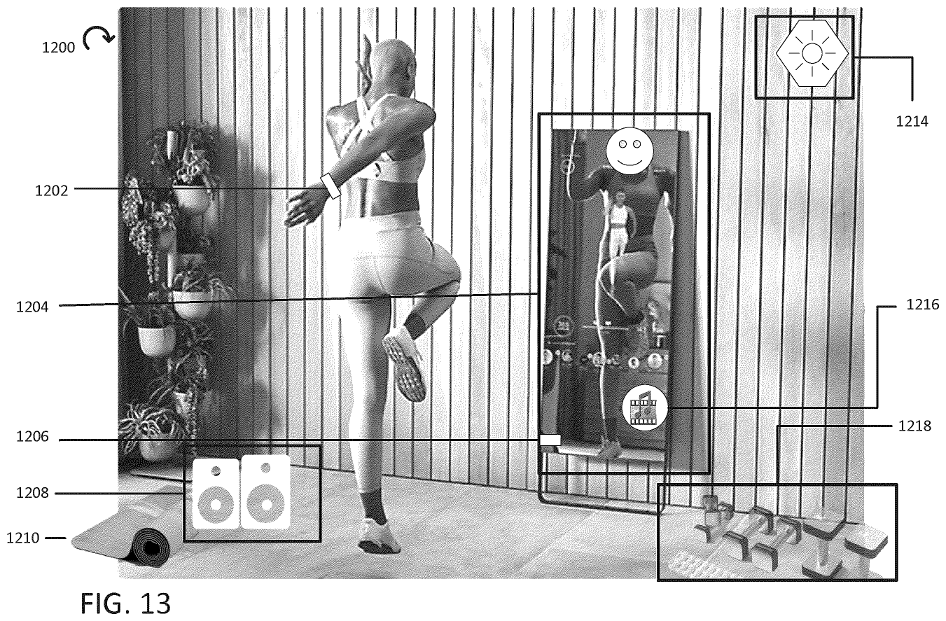

FIG. 13 shows the collection of user data and/or provision of individualized wellness pattern aware interventions within a smart system 1200 in accordance with an embodiment. FIG. 13 shows an example smart system 1200 embodiment. In this example embodiment, a number of user devices 10 and input devices 18 and output devices 19 may be involved in the methods and systems for providing an individualized wellness pattern aware intervention.

Types of smart exercise devices include smart mirror device, smart treadmill device, smart stationary bicycle device, smart home gym device, smart weight device, smart weightlifting device, smart bicycle device, smart exercise mat device, smart rower device, smart elliptical device, smart vertical climber, smart swim machine, smart boxing gym, smart boxing bag, smart boxing dummy, smart grappling dummy, smart dance studio, smart dance floor, smart dance barre, smart balance board, smart slide board, smart spin board, smart ski trainer, smart trampoline, or smart vibration platform. Additional smart devices that can be used in such a system include a connected music system, a connected lighting system, a connected heating device, a connected cooling device, a connected smell diffuser device, a connected electrical stimulation device, a connected implanted medical device. User in such systems may also input data and/or receive interventions through different devices such as a heart rate monitor, breathing monitor, a blood glucose monitor, galvanic skin response (GSR) monitor, an electronic implant, an EEG, a brain-computer interface. A hologram projection system, an autostereoscopic projection system virtual reality headset, an augmented reality headset, mixed reality devices, virtual reality devices, an augmented reality device, a metaverse headset, a haptic glove, a game controller, a haptic garment, which may or may not be integrated in other devices.

Contextual data which may be used to determine the intervention context, individualization, a method of providing a wellness pattern aware intervention, wearable biometric measurement smart watch device 1202 and audio video sensor 1206 receive data concerning the user's baseline activity, user's response to interventions. This data may be further interpreted in different ways, such as through facial recognition and audio analysis to evaluate an intervention. These devices may also provide the user with an intervention itself, or provide input to intervention tools, feedback tools, reflections tools such as chat responses, surveys, and monitoring interactions to provide an intervention, communicates with a smart device 302 which comprises controller 24. In this example, smart device 1204 is a smart mirror which includes a video audio input 1206.

In FIG. 13, smart system 1200 contains smart devices such as a smart exercise mirror 1204, smart yoga mat 1210, smart hand weights 1218, smart speakers 1208 and smart lighting 1214 which are provided as examples of smart devices found in a smart system 1200 which may provide an intervention in accordance with embodiments described herein. Interventions may be provided by shifting the environment using a smart device. As will be appreciated, a wide range of subtle environmental interventions have been shown to effect a change in user activity behaviour. Such interventions may include adjusting the lighting brightness or color of smart light 1214, adjusting the music played or the volume of the music on speaker 1208, adjusting the temperature of a connected exercise mat 1210, changing the color of a rep indicator on a smart weight 1218, and the like.

An intervention may be provided through one or more of a web application, an application installed on a user device, a smart mirror device, a connected music system, a connected lighting system, a connected exercise mat, a connected heating device, a connected cooling device, a connected smell diffuser device, a connected electrical stimulation device, a connected implanted medical device, a virtual reality headset, an augmented reality headset, a metaverse headset, mixed reality devices, virtual reality devices, an augmented reality devices, a haptic glove, a game controller, a haptic garment, a retail application, a coaching application, a fitness class or studio application, a retail application, a meal or nutritional supplement delivery service, an email system, a text message system, notification system, augmented reality environment, simulated reality environment, virtual reality environment, mixed reality environment, a game environment, a metaverse environment, and the like.

In FIG. 13 media player integrated component 1216 within smart mirror 1204 provides device or method of embedding a media aspect of in an intervention in accordance with an embodiment. Media player integrated component this component provides interventions that include interventions such as displaying specific video, motivational text, or color backgrounds behind or adjacent to an instructor; in one embodiment a short fun reward video is displayed when the user achieves a new success level; in one embodiment an intervention may be provided that includes engaging or anxiety inducing music when a user's eye focus shifts from the instruction as detected by audio video sensor 1206 or another way.

Media player integrated component 1216 within smart mirror 1204 may provide interventions that include providing one or more media of the type video, interactive presentation, game, image, hologram image projection, autostereoscopic image projection, audio, text, spoken word, guided conversation, music, interactive simulation wherein the media contains content with a greater than average statistical probability to result in one or more of a technique correction, an emotional shift, an intellectual reframing of an experience, an emotional reframing of an experience, a distraction from a current or past experience, changing the user's anxiety level, changing the user's fear level, changing the user's hopefulness level, changing the user's sense of competence, changing the user's curiosity level, changing the user's feeling of agency, offering a relatable motivational experience, changing the user's competitiveness level, changing the user's cooperation level, changing the user's perception of social connection, changing the user's perception of personal advancement, changing the user's perception of social status, changing the user's perception of social belonging, changing the user's gratitude level, changing the user's calmness level, changing the user's focus level, changing the user's equanimity level, changing the user's sense of inner peace level. For example, in one embodiment specific video, motivational text, or color backgrounds are displayed behind an instructor; in one embodiment a short fun reward video is displayed when the user achieves a new success level; in one embodiment engaging or anxiety inducing music is provided when a user's eye focus shifts from the instruction.

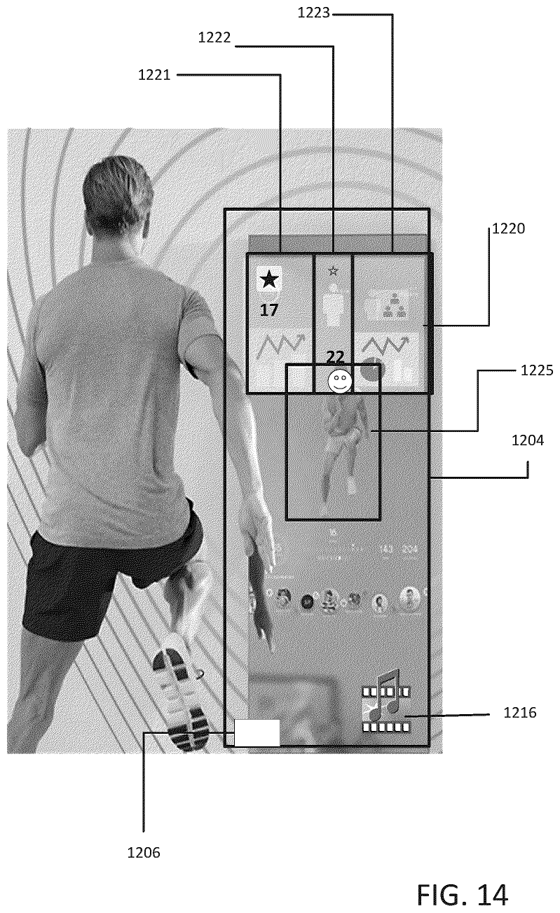

FIG. 14 shows an example embodiment in which individualized wellness pattern aware interventions may be displayed on an application 15 on a smart mirror 1204.

In this example, our athlete Chad is not meeting his performance targets and is distracted. The intervention system evaluates Chad's current context, activity, and potential interventions and determines providing feedback to Chad about his performance is highly probable to shift Chad's engagement with his workout to better correspond with Chad's goals and the wellness pattern that underlies his individualized interventions.

In this example, the individualized intervention provides Chad with a performance overlay 1220 on a smart mirror 1204. The performance overlay 1220 displays information about Chad's performance 1221 including current reps, and performance over the workout. Additional information is provided about another individual for comparison purposes 1222, in this case the individual is Chad's selected hero Chelsey who has completed more reps. As will be appreciated, any number of comparative statistics and visualizations may be displayed or provided as an individualized intervention. Chad could be provided with comparisons with other individuals, such as his instructor, Chad's ideal self, previous own best performance, recent own best performance, or the top athlete in his class, the top athlete throughout time, the top performance historically, the top performance within a specific time duration, an accountability partner, a digital twin, or the like. A community comparison 1223 is provided within performance overlay 1220, in this example, his fitness class. Community comparison 1223 may provide feedback about belonging and fitting in and/or ranking within the community. In one embodiment, accountability partners are able to view one another's performance metrics. Looking at performance overlay 1220 community comparison 1223, Chad is able to determine his ranking in his current fitness class.

In an embodiment the community comparison 1223 is primarily collaborative, rather than primarily competitive, in orientation. For example, individuals may contribute to a total community value for rep counts, excellent form, intensity of activity, focus, physiological coherence, and the like. In an embodiment, users work together towards a shared group target such as collectively burning a certain number of calories, staying in a HR range for a certain duration, working out for a certain duration, performing a perceived effort, or other metrics that account for activity, for example accumulating the steps/distance to climb mount Everest together, and the like.

In an embodiment, the community comparison 1223 defines collaborative sub-teams within the larger community which in one embodiment may be in competition, collaboration, and/or a combination of competition and collaboration with other sub-teams. In an embodiment, rewards, recognition, points, and the like may be associated with a community and/or sub-team. In an embodiment, a user is provided with an option to compete either against themselves and/or against others. In an embodiment, the user is provided with an option to participate independently or collaborate with others and to have a performance overlay 1220 that reflects their preferred option. In an embodiment, the performance overlay 1220 determination is associated with an individual based on a competitiveness scale rating, a player orientation to self or others scale rating, and/or a combination of competitiveness rating and self-other orientation rating and/or an activity, activity type, or activity category affiliation with a competitiveness scale rating, orientation to self or others, and or a combination of a competitiveness rating and self-other orientation rating. As will be appreciated, the specific feedback and presentation of that feedback, supports a number of different intervention types and may be associated with triggering a number of different feel states.

Individualized interventions may provide feedback or an indication related an activity being started, an activity in progress, an activity successfully completed, an activity duration, a user activity measure, a user activity success measure, a user activity partial success measure, a user activity in the context of a user's previous activity, a user activity in the context of a community activity, a user activity in the context of a digital twin data model, a user activity in the context of an ideal self data model, a user activity in the context of a cohort activity, a user activity in the context of a hero activity, a user activity in the context of a group of fitness class participants, a user's readiness to advance in an activity, a user's readiness to add a new activity, a user's readiness to stop an activity, a user's readiness to disrupt an activity, providing feedback or an indication related to at least one of a user habit being started, a user habit in progress, a user habit successfully completed, a user habit duration, a user habit activity measure, a user habit success measure, a user habit partial success measure, a user habit streak, a suggestion for resetting a habit, a suggestion for rescheduling a habit, rescheduling an activity, scheduling an activity, canceling a scheduled activity, a user's readiness to expand a habit, a user's readiness to add a new habit, a user's need to disrupt a habit, a user habit in the context of a community habit, a user habit in the context of a digital twin data model, a user habit in the context of an ideal self data model, a user habit in the context of a cohort habit, a user habit in the context of a hero habit, a user habit in the context of a group of fitness class participants.

Such feedback, indications, comparative context information may be combined with other aspects of an intervention. For example, an individualized intervention providing motivational feedback that the user has taken the lead position in a community may be accompanied by connected lights flashing or media player integrated component 1216 playing a short victory video. For example, an individualized intervention providing motivational feedback that the user's team has achieved a milestone in collaboration may be accompanied by an ability to send rewards to other team members, send encouragement to users on another team, open a live-chat function, send emoticons or messages, unlock a team opportunity, unlock a team reward, unlock a team badge, receive a shared invitation, activity, or social experience.

For example, in addition to the performance overlay, this motivational intervention for Chad includes adjustments to his workout content and his instructor's avatar 1225.

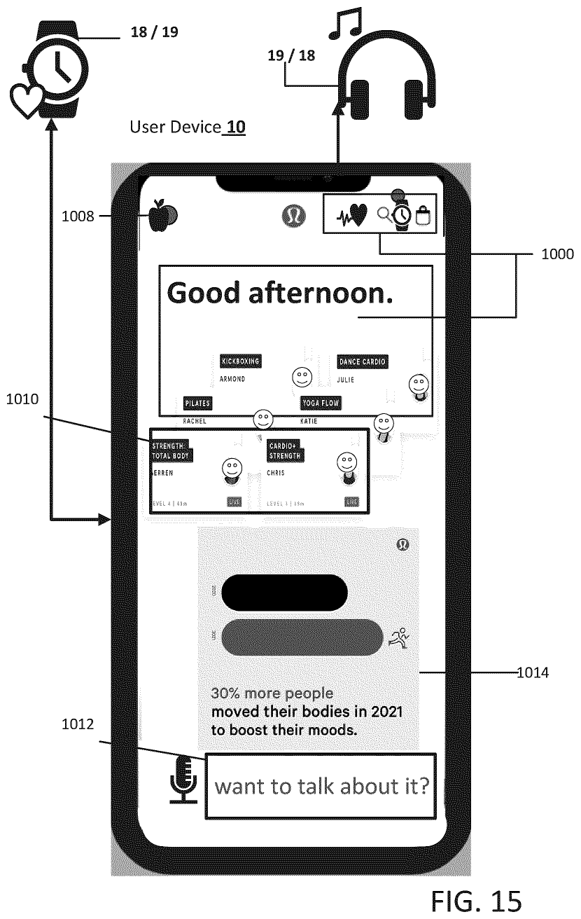

FIG. 15 shows an example embodiment in which individualized wellness pattern aware interventions may be displayed on an application 15 on user device 10. Interventions may also be provided within a conversational interaction, a notification, a pop-up suggestion, an alarm, a badge, a reward, a number of points, a retail application, a meal or nutritional supplement delivery service, an email system, a text message system, notification system, within one or more other applications on user device 10.

For the purposes of illustration, user device 10 has a heart rate monitor smart watch 18/19 and headphone with microphone 19/18 attached that both provide inputs to the system and method of providing interventions in addition to those displayed visually on user device 10.

In this example standard user interface, not arising from an intervention include notifications 1000 and a portion of the class offerings 1000. Part of the class offerings interface 1010 functions as an intervention, rather than as generic or customized content. In this case, our example user Fred has been attending Cardio workouts which are not consistent with the strength building component within the wellness pattern which is applied to their interventions. When the system evaluates Fred's current activity against the intended longer duration wellness pattern, an individualized intervention is generated that displays the strength building focused class offerings (Strength Total Body, Cardio-Strength) most prominently in their widget of class offerings. Fred is not aware that the system is intervening and nudging their activity by drawing their focus to classes that better support their wellness goals.

In an embodiment of the invention, application 15 on user device 10 includes a retail application. An intervention may include suggesting a product. Suggesting a product may include suggesting a product associated with a user's current, planned, or completed activities. In one embodiment, when a user selects and/or purchases a product, an intervention associated with the purchase context may be proposed. Suggesting a product includes suggesting one or more of a class, fitness accessory, item of apparel or the like. In an embodiment, an intervention suggests a product for the user to purchase for themselves, an accountability partner, a team member, a hero, an opposing team member, or the like.

In addition to other activity and behavioral indicators, biometric and GPS indicators from Fred's smart watch device suggest Fred may have skipped lunch. A number of potential interventions are possible. Historically Fred has not responded to more explicit audio interventions, or notifications, prompting them to eat. The intervention 1080 provides a small visual reminder which has a historical success rate of encouraging others in similar cohorts and communities to engage in eating. This intervention is now being provided to Fred to attempt to adjust their activity and evaluate the efficacy of this individualized intervention type for them.

Content 1014 may contain content data, an individualized intervention, or customized content in accordance with embodiments.

In this example, in addition to skipping lunch, Fred has quit their yoga class before completion. Fred's patterns today provide a context that suggests a reflective chat intervention might be well received and helpful. The Want to chat about it? 1012 intervention is displayed in the application as an individualized intervention to nudge Fred to reflect on what is happening.

As is evident in these examples, a number of activity contexts, including data and metadata inputs, may be evaluated to determine one or more intervention to offer to a user. In one embodiment, these potential interventions are then evaluated based on wellness patterns 410, individualization 412, intervention 414, in the context of cohort and digital twin simulations and this modeling informs the generating individualized wellness pattern aware interventions.

The word “a” or “an” when used in conjunction with the term “comprising” or “including” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

The terms “coupled”, “coupling” or “connected” as used herein can have several different meanings depending on the context in which these terms are used. For example, the terms coupled, coupling, or connected can have a mechanical or electrical connotation. For example, as used herein, the terms coupled, coupling, or connected can indicate that two elements or devices are directly connected to one another or connected to one another through one or more intermediate elements or devices via an electrical element, electrical signal or a mechanical element depending on the particular context. The term “and/or” herein when used in association with a list of items means any one or more of the items comprising that list.

As used herein, a reference to “about” or “approximately” a number or to being “substantially” equal to a number means being within +/−10% of that number.

The technical solution of embodiments may be in the form of a software product. The software product may be stored in a non-volatile or non-transitory storage medium. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

While the disclosure has been described in connection with specific embodiments, it is to be understood that the disclosure is not limited to these embodiments, and that alterations, modifications, and variations of these embodiments may be carried out by the skilled person without departing from the scope of the disclosure.

It is furthermore contemplated that any part of any aspect or embodiment discussed in this specification can be implemented or combined with any part of any other aspect or embodiment discussed in this specification.

1. A non-transitory computer readable medium with instructions stored thereon, that when executed by a hardware processor cause the processor to generate output data indicating an individualized wellness pattern aware intervention and provide a user device with the individualized wellness pattern aware intervention to activate, trigger or present the intervention, wherein the processor generates the intervention by applying a plurality of computer models to an individualization data object generated by processing input data identifying qualities associated with the intervention and additional input data identifying qualities associated with the intervention from a simulation environment with one or more digital twins corresponding to one or more physical objects relating to the intervention, wherein the instructions control the hardware processor to trigger measurements by one or more input devices to receive at least part of the input data.

2. (canceled)

3. The non-transitory computer readable medium of claim 1 wherein the additional input data comprises data relating to at least one of mood and emotion within the simulation environment.

4. The non-transitory computer readable medium of claim 1 wherein the measurements comprise data relating to physical physiological modeling.

5. The non-transitory computer readable medium of claim 1 wherein the plurality of computer models comprise a wellness pattern model, an individualization model, and an intervention model.

6. The non-transitory computer readable medium of claim 1 wherein the individualized wellness pattern aware intervention comprises a multi-feel state longitudinal journey over a multi-day time duration to create a specific type of user experience over the multi-day time duration.

7. The non-transitory computer readable medium of claim 1 wherein the individualized wellness pattern aware intervention comprises an intervention output that is associated with an intervention type.

8. The non-transitory computer readable medium of claim 7 wherein the intervention type comprises one or more of suggesting an activity, starting an activity, suggesting a change in a current activity, changing a current activity, suggesting a change in the intensity of an activity, changing the intensity of an activity, canceling a scheduled activity, suggesting canceling a scheduled activity, suggesting the end of a current activity, ending a current activity, providing a reward, increasing a challenge, suggesting a product, providing a product, providing points, suggesting a future activity, starting a future activity, displaying a tool for user reflection, suggesting user feedback, displaying a tool for user feedback, suggesting a user chat or conversational activity, opening a user chat, suggesting an accountability partner, providing an accountability partner, suggesting a change in a current accountability partner, changing a current accountability partner, suggesting a team, providing a team, suggesting a change in a current team, changing a current team, suggesting a community, providing a community, suggesting a change in a current community, changing a current community, providing a level, removing a level, customizing a level, upgrading a level, downgrading a level, providing access to a product, providing access to an event, providing access to an experience, providing a badge, changing a badge, changing a social ranking, changing an indication of social belonging, changing an indication of contribution to a group, team or community, changing a social network connection, changing a group membership, providing a social connection, providing a means of communicating with another user, providing a means of sharing an emoticon with another user, providing feedback or an indication related to at least one of an activity being started, an activity in progress, an activity successfully completed, an activity duration, a user activity measure, a user activity success measure, a user activity partial success measure, a user activity in the context of a user's previous activity, a user activity in the context of a community activity, a user activity in the context of a digital twin data model, a user activity in the context of an ideal self data model, a user activity in the context of a cohort activity, a user activity in the context of a hero activity, a user activity in the context of a group of fitness class participants, a user activity in the context of an accountability partner, a user activity in the context of team, a user activity in the context of a community, a user's readiness to advance in an activity, a user's readiness to add a new activity, a user's readiness to stop an activity, a user's readiness to disrupt an activity, providing feedback or an indication related to at least one of a user habit being started, a user habit in progress, a user habit successfully completed, a user habit duration, a user habit activity measure, a user habit success measure, a user habit partial success measure, a user habit streak, a suggestion for resetting a habit, a suggestion for rescheduling a habit, rescheduling an activity, scheduling an activity, canceling a scheduled activity, a user's readiness to expand a habit, a user's readiness to add a new habit, a user's need to disrupt a habit, a user habit in the context of a community habit, a user habit in the context of a digital twin data model, a user habit in the context of an ideal self data model, a user habit in the context of a cohort habit, a user habit in the context of a hero habit, a user habit in the context of a group of fitness class participants, providing a virtual assistant, customizing a virtual assistant, providing information about a product, providing personalized information about a product, providing customized information about a product, providing a navigational path, providing a customized navigational path, providing a category of products, providing a customized category of products, and filtering a set of products, providing a special offer.

9. The non-transitory computer readable medium of claim 8 further comprising providing one or more media of the type video, interactive presentation, game, image, hologram image projection, autostereoscopic image projection, audio, text, spoken word, guided conversation, music, interactive simulation wherein the media contains content with a greater than average statistical probability to result in one or more of a technique correction, an emotional shift, an intellectual reframing of an experience, an emotional reframing of an experience, a distraction from a current or past experience, changing the user's anxiety level, changing the user's fear level, changing the user's hopefulness level, changing the user's sense of competence, changing the user's curiosity level, changing the user's feeling of agency, offering a relatable motivational experience, changing the user's competitiveness level, changing the user's cooperation level, changing the user's perception of social connection, changing the user's perception of personal advancement, changing the user's perception of social status, changing the user's perception of social belonging, changing the user's gratitude level, changing the user's calmness level, changing the user's focus level, changing the user's equanimity level, changing the user's sense of inner peace level.

10. The non-transitory computer readable medium of claim 6 wherein the multi-feel state longitudinal journey designed to create a specific type of user experience over a multi-day time duration is associated with one or more of the archetypal pattern wherein the archetypal pattern is one of, a quest for identity, a quest to find a ideal location, emotional state, or sense of spiritual realization, a quest for justice, a quest to help a community member, a quest for social connection, a quest for social acceptance within a group, a quest for a role or status designation, a quest in search of knowledge, competence or skill, a quest for acceptance and personal affirmation, a quest for transformation, a quest for self-actualization, a quest for pleasure, fun, surprise and adventure, a quest to remove a danger, a quest for a symbolic or metaphoric goal.

11. The non-transitory computer readable medium of claim 6 wherein the multi-feel state longitudinal journey designed to create a specific type of user experience over a multi-day time duration is associated with a pattern for adherence to a wellness, fitness lifestyle, basic health criteria, training plan, nutritional plan, and interventions are provided that are consistent with generating and improving the individual's adherence to the overall plan.

12. The non-transitory computer readable medium of claim 1 wherein the individualized wellness pattern aware intervention contains an interactive element or selectable indicia that enable a user to engage with and/or select from more than one individualized wellness pattern aware intervention.

13. The non-transitory computer readable medium of claim 1 wherein the individualized wellness pattern aware intervention is generated as a module with executable instructions to receive inputs, provide outputs and display the habit aware intervention.

14. A computer system for providing a user device with an individualized wellness pattern aware intervention for a specific type of multi-feel state longitudinal journey over a time duration, the system comprising:
a communication interface to transmit the individualized wellness pattern aware intervention;
one or more non-transitory memory;
a hardware processor programmed with executable instructions for generating the individualized wellness pattern aware intervention, wherein the hardware processor:
receives individualization input data associated with a user;
generates an individualization data object using the input data and by processing additional input data identifying qualities associated with the intervention from a simulation environment with one or more digital twins corresponding to one or more physical objects relating to the intervention;
determines one or more intervention types to generate based on the data object by applying a plurality of computer models to the data object;
generates one or more individualized wellness pattern aware intervention using the intervention types;
one or more input devices to perform measurements for at least part of the input data;
a user device comprising a hardware processor, and an interface to receive the intervention, and activate, trigger or present the intervention.

15.-27. (canceled)

28. A computer implemented method for providing a user device with an individualized wellness pattern aware intervention for a specific type of multi-feel state longitudinal journey over a time duration, the method comprising:
transmitting the individualized wellness pattern aware intervention using a communication interface;
generating the individualized wellness pattern aware intervention using hardware processor programmed with executable instructions by:
receiving individualization input data associated with a user;
generating an individualization data object using the input data and by processing additional input data identifying qualities associated with the intervention from a simulation environment with one or more digital twins corresponding to one or more physical objects relating to the intervention;
determining one or more intervention types to generate based on the data object by applying a plurality of computer models to the data object;
generating one or more individualized wellness pattern aware intervention using the intervention types;
performing measurements using one or more input devices to for at least part of the input data; and
receiving the intervention at user device and activating, triggering or presenting the intervention.

29.-52. (canceled)

53. The computer system of claim 14,
wherein the one or more non-transitory memory stores a trained individualization model, a trained wellness pattern model, and a trained intervention model;
wherein the hardware processor is programmed with the executable instructions in the non-transitory memory to evaluate at least one model of the trained individualization model, the trained wellness pattern model, and the trained intervention model, evaluate an activity, evaluate the intervention, and evaluate a modification to the intervention to generate the one or more individualized wellness pattern aware intervention.

54-58. (canceled)

59. The system of claim 53 wherein the hardware processor updates one or more of the trained individualization model, trained wellness pattern model, trained intervention model stored in the memory based on machine learning, wherein the machine learning is based on simulated digital twin intervention feedback.

60. (canceled)

61. The system of claim 53, wherein the intervention model comprises an activity measure comprising evaluation of activity effect using a digital twin model.

62-89. (canceled)

90. The computer system of claim 14 wherein the individualized wellness pattern aware intervention comprises a multi-feel state longitudinal journey over a multi-day time duration for a specific type of user experience over the multi-day time duration.

91. The computer system of claim 90 wherein the multi-feel state longitudinal journey for the specific type of user experience over the multi-day time duration is associated with one or more of the archetypal pattern wherein the archetypal pattern is one of, a quest for identity, a quest to find a ideal location, emotional state, or sense of spiritual realization, a quest for justice, a quest to help a community member, a quest for social connection, a quest for social acceptance within a group, a quest for a role or status designation, a quest in search of knowledge, competence or skill, a quest for acceptance and personal affirmation, a quest for transformation, a quest for self-actualization, a quest for pleasure, fun, surprise and adventure, a quest to remove a danger, a quest for a symbolic or metaphoric goal.

92. The computer system of claim 90 wherein the multi-feel state longitudinal journey designed to create a specific type of user experience over a multi-day time duration is associated with a pattern for adherence to a wellness, fitness lifestyle, basic health criteria, training plan, nutritional plan, and interventions are provided that are consistent with generating and improving the individual's adherence to the overall plan.
